SEQUENCE LISTING (99 sequences)

SEQ ID NO: 1 - protein, 10 aa, Artificial Sequence
  Ab Nos. 1, 7, 8, 9, 15 VL-CDR1
KSVSTSGYSY

SEQ ID NO: 2 - protein, 3 aa, Artificial Sequence
  Ab Nos. 1, 6, 7, 8, 9, 15 VL-CDR2
LVS

SEQ ID NO: 3 - protein, 8 aa, Artificial Sequence
  Ab Nos. 1, 7, 8, 9, 15 VL-CDR3
QHIRELTR

SEQ ID NO: 4 - protein, 8 aa, Artificial Sequence
  Ab No. 1 VH-CDR1
GYTFTDYY

SEQ ID NO: 5 - protein, 8 aa, Artificial Sequence
  Ab No. 1 VH-CDR2
INPYNGGT

SEQ ID NO: 6 - protein, 13 aa, Artificial Sequence
  Ab No. 1 VH-CDR3
ARSYRYDRAWFAY

SEQ ID NO: 7 - protein, 11 aa, Artificial Sequence
  Ab No. 6 VL-CDR1
QSLLDSDGKTY

SEQ ID NO: 8 - protein, 9 aa, Artificial Sequence
  Ab No. 6 VL-CDR3
WQGTHFPRT

SEQ ID NO: 9 - protein, 17 aa, Artificial Sequence
  Ab No. 6 VH-CDR1
YTFSRRVYFAIRDTNYW

SEQ ID NO: 10 - protein, 8 aa, Artificial Sequence
  Ab No. 6 VH-CDR2
IYPGNGDT

SEQ ID NO: 11 - protein, 10 aa, Artificial Sequence
  Ab No.6 VH-CDR3
ACDYYYAMDY

SEQ ID NO: 12 - protein, 8 aa, Artificial Sequence
  Ab No. 7 VH-CDR1
GFTFSDYR

SEQ ID NO: 13 - protein, 10 aa, Artificial Sequence
  Ab No. 7 VH-CDR2
IKRKSDNYGT

SEQ ID NO: 14 - protein, 10 aa, Artificial Sequence
  Ab No. 7 VH-CDR3
GRGLAEGFPY

SEQ ID NO: 15 - protein, 8 aa, Artificial Sequence
  Ab No. 8 VH-CDR1
GFTFSNYA

SEQ ID NO: 16 - protein, 8 aa, Artificial Sequence
  Ab No. 8 VH-CDR2
ISSGGSYT

SEQ ID NO: 17 - protein, 8 aa, Artificial Sequence
  Ab No. 8 VH-CDR3
AREGWEGY

SEQ ID NO: 18 - protein, 8 aa, Artificial Sequence
  Ab No. 9 VH-CDR1
GFTFSDYY

SEQ ID NO: 19 - protein, 8 aa, Artificial Sequence
  Ab No. 9 VH-CDR2
ISDGGSYT

SEQ ID NO: 20 - protein, 18 aa, Artificial Sequence
  Ab No. 9 VH-CDR3
ARRGLLNSLLRPPYAMDY

SEQ ID NO: 21 - protein, 6 aa, Artificial Sequence
  Ab No. 11 VL-CDR1
QSISNN

SEQ ID NO: 22 - protein, 3 aa, Artificial Sequence
  Ab Nos. 11, 14 VL-CDR2
YAS

SEQ ID NO: 23 - protein, 9 aa, Artificial Sequence
  Ab No. 11 VL-CDR3
QQSNSWPLT

SEQ ID NO: 24 - protein, 8 aa, Artificial Sequence
  Ab No. 11 VH-CDR1
VYTFTSYW

SEQ ID NO: 25 - protein, 8 aa, Artificial Sequence
  Ab No. 11 VH-CDR2
IFPGTGTT

SEQ ID NO: 26 - protein, 14 aa, Artificial Sequence
  Ab No. 11 VH-CDR3
ARYGGNQYYYAMDY

SEQ ID NO: 27 - protein, 12 aa, Artificial Sequence
  Ab No. 12 VL-CDR1
QSLLNSGNQKSY

SEQ ID NO: 28 - protein, 3 aa, Artificial Sequence
  Ab Nos. 12, 18 VL-CDR2
WAS

SEQ ID NO: 29 - protein, 9 aa, Artificial Sequence
  Ab Nos. 12, 18 VL-CDR3
QNDYSYPLT

SEQ ID NO: 30 - protein, 9 aa, Artificial Sequence
  Ab No. 12 VH-CDR1
GYSITSDYA

SEQ ID NO: 31 - protein, 7 aa, Artificial Sequence
  Ab No. 12 VH-CDR2
ISYSGST

SEQ ID NO: 32 - protein, 14 aa, Artificial Sequence
  Ab No. 12 VH-CDR3
ARQTYYYGSSPFTY

SEQ ID NO: 33 - protein, 6 aa, Artificial Sequence
  Ab No. 14 VL-CDR1
QSVSND

SEQ ID NO: 34 - protein, 9 aa, Artificial Sequence
  Ab No. 14 VL-CDR3
QQDYSSPYT

SEQ ID NO: 35 - protein, 8 aa, Artificial Sequence
  Ab No. 14 VH-CDR1
GFSLTSYG

SEQ ID NO: 36 - protein, 8 aa, Artificial Sequence
  Ab No. 14 VH-CDR2
IWRGGSTD

SEQ ID NO: 37 - protein, 13 aa, Artificial Sequence
  Ab No. 14 VH-CDR3
AKKKYGNYGAMDY

SEQ ID NO: 38 - protein, 8 aa, Artificial Sequence
  Ab No. 15 VH-CDR1
GYTFSSYW

SEQ ID NO: 39 - protein, 8 aa, Artificial Sequence
  Ab No. 15 VH-CDR2
ILPGSGST

SEQ ID NO: 40 - protein, 3 aa, Artificial Sequence
  Ab No. 15 VH-CDR3
ATT

SEQ ID NO: 41 - protein, 12 aa, Artificial Sequence
  Ab No. 18 VL-CDR1
QSLLNSGNQKNY

SEQ ID NO: 42 - protein, 8 aa, Artificial Sequence
  Ab No. 18 VH-CDR1
GHTFTSYW

SEQ ID NO: 43 - protein, 8 aa, Artificial Sequence
  Ab No. 18 VH-CDR2
IAPGSGST

SEQ ID NO: 44 - protein, 13 aa, Artificial Sequence
  Ab No. 18 VH-CDR3
ARNLKYGNYGFAY

SEQ ID NO: 45 - DNA, 329 bp, Artificial Sequence
  Ab No. 1 Light Chain (332-1)_A1-T7_JZ nucleotide sequence (329 bp)
gacattgtgatgtcacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggagatcaaa

SEQ ID NO: 46 - protein, 109 aa, Artificial Sequence
  Ab No. 1 Light Chain (332-1)_A1-T7_JZ amino acid sequence
DIVMSQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWRS

SEQ ID NO: 47 - DNA, 359 bp, Artificial Sequence
  Ab No. 1 Heavy Chain (H1-1)_P17_PW nucleotide sequence (359bp)
aggtgcagcttcaggagtcaggacctgagctggtgaagcctggggcttcagtgaagatgtcctgtaaggcttctggatacacattcactgactactacatggactgggtgaagcagagccatggagaaagctttgagtggattggacgtattaatccttacaatggtggtactagctacaaccagaagttcaacgacaaggccacattgactgttgacaagtcctccagcacagcctacatggagctcaacagcctgacatctgaggactctgcggtctattactgtgcaagaagttataggtacgaccgagcctggtttgcttactggggccaagggaccacggtcaccgtctcctca

SEQ ID NO: 48 - protein, 119 aa, Artificial Sequence
  Ab No. 1 Heavy Chain (H1-1)_P17_PW amino acid sequence
VQLQESGPELVKPGASVKMSCKASGYTFTDYYMDWVKQSHGESFEWIGRINPYNGGTSYNQKFNDKATLTVDKSSSTAYMELNSLTSEDSAVYYCARSYRYDRAWFAYWGQGTTVTVSS

SEQ ID NO: 49 - DNA, 336 bp, Artificial Sequence
  Ab No. 6 Light Chain 313-3_F6-T7_JZ nucleotide sequence (336bp)
gacattgtgatgacacagtctccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcggacgttcggtggaggcaccaagctggaaataaaa

SEQ ID NO: 50 - protein, 112 aa, Artificial Sequence
  Ab No. 6 Light Chain 313-3_F6-T7_JZ amino acid sequence
DIVMTQSPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 51 - DNA, 377 bp, Artificial Sequence
  Ab No. 6 Heavy Chain (K1-6)_P17_PW nucleotide sequence (377bp)
aggtgcagctggaggagtctgggcctgagctggcaaggccttgggcttcagtgaagatatcctgccaggctttctacaccttttccagaagggtgtactttgccattagggataccaactactggatgcagtgggtaaaacagaggcctggacagggtctggaatggatcggggctatttatcctggaaatggtgatactagttacaatcagaagttcaagggcaaggccacattgactgcagacaaatcctccagcacagcctacatgcaactcagcagcctgacatctgaggactctgcggtctattactgtgcatgtgattactactatgctatggactactggggccaagggaccacggtcaccgtctcctca

SEQ ID NO: 52 - protein, 125 aa, Artificial Sequence
  Ab No. 6 Heavy Chain (K1-6)_P17_PW amino acid sequence
VQLEESGPELARPWASVKISCQAFYTFSRRVYFAIRDTNYWMQWVKQRPGQGLEWIGAIYPGNGDTSYNQKFKGKATLTADKSSSTAYMQLSSLTSEDSAVYYCACDYYYAMDYWGQGTTVTVSS

SEQ ID NO: 53 - DNA, 329 bp, Artificial Sequence
  Ab No. 7 Light Chain (314-1)_F7-T7_JZ nucleotide sequence (329bp)
gatattgtgatgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggagctgaaa

SEQ ID NO: 54 - protein, 108 aa, Artificial Sequence
  Ab No. 7 Light Chain (314-1)_F7-T7_JZ amino acid sequence
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWS

SEQ ID NO: 55 - DNA, 356 bp, Artificial Sequence
  Ab No. 7 Heavy Chain (K1-7)_P17_PW nucleotide sequence (356bp)
aggtgcagctggtggagtctgggggaggcttggtgaggcctggaaattctctgaaactctcctgtgttacctcgggattcactttcagtgactaccggatgcactggcttcgccagcctccagggaagaggctggagtggattgctgtaattaaacgcaagtctgataattatggaacaaattatgcagagtctgtgaaaggcagattcactatttcaagagatgacgcagaaagcagtgtctacctgcagatgaacagaataagagaggacgacactgccacttattattgtggtagaggcctagccgaagggtttccttattggggccaagggaccacggtcaccgtctcctca

SEQ ID NO: 56 - protein, 118 aa, Artificial Sequence
  Ab No. 7 Heavy Chain (K1-7)_P17_PW amino acid sequence
VQLVESGGGLVRPGNSLKLSCVTSGFTFSDYRMHWLRQPPGKRLEWIAVIKRKSDNYGTNYAESVKGRFTISRDDAESSVYLQMNRIREDDTATYYCGRGLAEGFPYWGQGTTVTVSS

SEQ ID NO: 57 - DNA, 329 bp, Artificial Sequence
  Ab No. 8 Light Chain (335-1)_A8-T7_JZ nucleotide sequence (329bp)
gacattgtgatgtcacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaactaaaa

SEQ ID NO: 58 - protein, 108 aa, Artificial Sequence
  Ab No. 8 Light Chain (335-1)_A8-T7_JZ amino acid sequence
DIVMSQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWN

SEQ ID NO: 59 - DNA, 344 bp, Artificial Sequence
  Ab No. 8 Heavy Chain (K1-8)_P17_PW nucleotide sequence (344bp)
tggtgcagctggaggaatctgggggaggcttagtgaagcctggagggtccctgaaactctcctgtgcagcctctggattcactttcagtaactatgccatgtcttgggttcgccagactccggagaagaggctggagtgggtcgcaaccattagtagtggtggtagttacacctactatccagacagtgtgaagggtcgattcaccatctccagagacaatgccaagaacaccctgtacctgcaaatgagcagtctgaggtctgaggacacggccatgtattactgtgccagagagggctgggaaggctactggggccaagggaccacggtcaccgtctcctca

SEQ ID NO: 60 - protein, 114 aa, Artificial Sequence
  Ab No. 8 Heavy Chain (K1-8)_P17_PW amino acid sequence
VQLEESGGGLVKPGGSLKLSCAASGFTFSNYAMSWVRQTPEKRLEWVATISSGGSYTYYPDSVKGRFTISRDNAKNTLYLQMSSLRSEDTAMYYCAREGWEGYWGQGTTVTVSS

SEQ ID NO: 61 - DNA, 329 bp, Artificial Sequence
  Ab No. 9 Light Chain (336-1)_A9-T7_JZ nucleotide sequence (329bp)
gacattgtgatgtcacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatgaaa

SEQ ID NO: 62 - protein, 108 aa, Artificial Sequence
  Ab No. 9 Light Chain (336-1)_A9-T7_JZ amino acid sequence
DIVMSQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 63 - DNA, 374 bp, Artificial Sequence
  Ab No. 9 Heavy Chain (K1-9)_P17_PW nucleotide sequence (374bp)
aggtgcagctggtggagtctgggggaggcttagtgaagcctggagggtccctgaaactctcctgtgcagcctctggattcactttcagtgactattacatgtattgggttcgccagactccggaaaagaggctggagtgggtcgcaaccattagtgatggtggtagttacacctactatccagaccgtgtgaaggggcgattcaccatctccagagacaatgccaagaacaacctgtacctgcaaatgagcagtctgaagtctgaggacacagccatgtattactgtgcaagacgggggcttctgaattcattactacggcccccctatgctatggactactggggccaagggaccacggtcaccgtctcctca

SEQ ID NO: 64 - protein, 124 aa, Artificial Sequence
  Ab No. 9 Heavy Chain (K1-9)_P17_PW amino acid sequence
VQLVESGGGLVKPGGSLKLSCAASGFTFSDYYMYWVRQTPEKRLEWVATISDGGSYTYYPDRVKGRFTISRDNAKNNLYLQMSSLKSEDTAMYYCARRGLLNSLLRPPYAMDYWGQGTTVTVSS

SEQ ID NO: 65 - DNA, 321 bp, Artificial Sequence
  Ab No. 11 Light Chain (317-4)_F11-T7_JZ nucleotide sequence (321bp)
gatattgtgatgacacagtctccagccaccctgtctgtgactccaggagatagcgtcagtctttcctgcagggccagccaaagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggcctctcacgttcggtgctggcaccaagctggaactgaaa

SEQ ID NO: 66 - protein, 107 aa, Artificial Sequence
  Ab No. 11 Light Chain (317-4)_F11-T7_JZ amino acid sequence
DIVMTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 67 - DNA, 362 bp, Artificial Sequence
  Ab No. 11 Heavy Chain (K1-11)_P17_PW nucleotide sequence (362bp)
aggtgcagctggtggagtctggagctgagctggtgaagcctggggcttcagtgaagctgtcctgcaagacttctgtctacaccttcaccagctactggattcagtgggtaaaacagaggcctggacagggccttgggtggattggagagatatttcctggaactggcactacttactacaatgagaagttcaagggcaaggccacactgactatagacacatcctccagcacagcctacatgcagctcagcagcctgacctctgaggactctgctgtctatttctgtgcaagatacggtggtaaccaatattactatgctatggactactggggccaagggaccacggtcaccgtctcctca

SEQ ID NO: 68 - protein, 120 aa, Artificial Sequence
  Ab No. 11 Heavy Chain (K1-11)_P17_PW amino acid sequence
VQLVESGAELVKPGASVKLSCKTSVYTFTSYWIQWVKQRPGQGLGWIGEIFPGTGTTYYNEKFKGKATLTIDTSSSTAYMQLSSLTSEDSAVYFCARYGGNQYYYAMDYWGQGTTVTVSS

SEQ ID NO: 69 - DNA, 339 bp, Artificial Sequence
  Ab No. 12 Light Chain (318-1)_F12-T7_JZ nucleotide sequence (339bp)
gatattgtgatgacacagactccatcctccctgactgtgacagcaggagagaaggtcactatgaggtgcaagtccagtcagagtctgttaaacagtggaaatcaaaagagctacttgacctggtaccagcagaaaccagggcagcctcctaaactgttgatctactgggcatccactagggaatctggggtccctgatcgcttcacaggcagtggatctggaaccgatttcactcttaccatcagcagtgtgcaggctgaagacctggcagtttattactgtcagaatgattatagttatccgctcacgttcggtgctgggaccaagctggaactcaaa

SEQ ID NO: 70 - protein, 113 aa, Artificial Sequence
  Ab No. 12 Light Chain (318-1)_F12-T7_JZ amino acid sequence
DIVMTQTPSSLTVTAGEKVTMRCKSSQSLLNSGNQKSYLTWYQQKPGQPPKLLIYWASTRESGVPDRFTGSGSGTDFTLTISSVQAEDLAVYYCQNDYSYPLTFGAGTKLELK

SEQ ID NO: 71 - DNA, 362 bp, Artificial Sequence
  Ab No. 12 Heavy Chain (H1-12)_P17_PW(2) nucleotide sequence (362bp)
aggtgcagcttcaggagtcaggacctggcctggtgaaaccttctcagtctctgtccctcacctgcactgtcactggctactcaatcaccagtgattatgcctggaactggatccggcagtttccaggaaacaaactggagtggatgggctacataagctacagtggtagcactagctacaacccatctctcaaaagtcgaatctctatcactcgagacacatccaagaaccagttcttcctgcacttgaattctgtgactactgaggacacagccacatattactgtgcaagacaaacctattactacggtagtagcccctttacttactggggccaagggaccacggtcaccgtctcctca

SEQ ID NO: 72 - protein, 120 aa, Artificial Sequence
  Ab No. 12 Heavy Chain (H1-12)_P17_PW(2) amino acid sequence
VQLQESGPGLVKPSQSLSLTCTVTGYSITSDYAWNWIRQFPGNKLEWMGYISYSGSTSYNPSLKSRISITRDTSKNQFFLHLNSVTTEDTATYYCARQTYYYGSSPFTYWGQGTTVTVSS

SEQ ID NO: 73 - DNA, 321 bp, Artificial Sequence
  Ab No. 14 Light Chain (319-1)_F14-T7_JZ nucleotide sequence (321bp)
gacattgtgatgacccagtctcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctccgtacacgttcggagggggcaccaagctggaactcaaa

SEQ ID NO: 74 - protein, 107 aa, Artificial Sequence
  Ab No. 14 Light Chain (319-1)_F14-T7_JZ amino acid sequence
DIVMTQSPKFLLVSAGDRVTITCKASQSVSNDVAWYQQKPGQSPKLLIYYASNRYTGVPDRFTGSGYGTDFTFTISTVQAEDLAVYFCQQDYSSPYTFGGGTKLELK

SEQ ID NO: 75 - DNA, 356 bp, Artificial Sequence
  Ab No. 14 Heavy Chain (H1-14)_P17_PW nucleotide sequence (356bp)
aggtgcagcttcaggagtcaggacctggcctagtgcagccctcacagagcctgtccataacctgcacagtctctggtttctcattaactagctatggtgtacactgggttcgccagtctccaggaaagggtctggagtggctgggagtgatatggagaggtggaagcacagactacaatgcagctttcatgtccagactgagcatcaccaaggacaactccaagagccaagttttctttaaaatgaacagtctgcaagctgatgacactgccatatactactgtgccaaaaagaagtatggtaactacggagctatggactactggggccaagggaccacggtcaccgtctcctca

SEQ ID NO: 76 - protein, 118 aa, Artificial Sequence
  Ab No. 14 Heavy Chain (H1-14)_P17_PW amino acid sequence
VQLQESGPGLVQPSQSLSITCTVSGFSLTSYGVHWVRQSPGKGLEWLGVIWRGGSTDYNAAFMSRLSITKDNSKSQVFFKMNSLQADDTAIYYCAKKKYGNYGAMDYWGQGTTVTVSS

SEQ ID NO: 77 - DNA, 329 bp, Artificial Sequence
  Ab No. 15 Light Chain (320-3)_F15_T7_JZ nucleotide sequence (329bp)
gacattgtgatgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggagatgaaa

SEQ ID NO: 78 - protein, 108 aa, Artificial Sequence
  Ab No. 15 Light Chain (320-3)_F15_T7_JZ amino acid sequence
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWR

SEQ ID NO: 79 - DNA, 328 bp, Artificial Sequence
  Ab No. 15 Heavy Chain (H1-15)_P17_PW nucleotide sequence (328bp)
aggtgcagctgcaggagtcaggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaactacaatgagaagtttaagggcaaggccacattcactgcagatacatcctccaacacagcgtacttgcaactcagcagcctgacatctgaggactctgccgtctattactgtgcgactactggggccaagggaccacggtcaccgtctcctca

SEQ ID NO: 80 - protein, 108 aa, Artificial Sequence
  Ab No. 15 Heavy Chain (H1-15)_P17_PW amino acid sequence
VQLQESGAELMKPGASVKISCKATGYTFSSYWIEWVKQRPGHGLEWIGEILPGSGSTNYNEKFKGKATFTADTSSNTAYLQLSSLTSEDSAVYYCATTGAKGPRSPSP

SEQ ID NO: 81 - DNA, 339 bp, Artificial Sequence
  Ab No. 18 Light Chain (321-3)_F18-T7_JZ nucleotide sequence (339bp)
gacattgtgatgacacagactccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctggcaccaagctggaaatcaaa

SEQ ID NO: 82 - protein, 113 aa, Artificial Sequence
  Ab No. 18 Light Chain (321-3)_F18-T7_JZ amino acid sequence
DIVMTQTPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIK

SEQ ID NO: 83 - DNA, 359 bp, Artificial Sequence
  Ab No. 18 Heavy Chain (K1-18)_P17_PW nucleotide sequence (359bp)
aggtgcagctggaggagtctggagatgatctggtaaagcctggggcctcagtgaagctgtcctgcaaggcttctggccacaccttcaccagctactggattaactggataaaacagaggcctggacagggccttgagtggataggacgtattgctcctggaagtggtagtacttactacaatgaaatgttcaagggcaaggcaacactgactgtagacacatcctccagcacagcctacattcagctcagcagcctgtcatctgaggactctgctgtctatttctgtgcaagaaacctaaagtatggtaactacgggtttgcttactggggccaagggaccacggtcaccgtctcctca

SEQ ID NO: 84 - protein, 119 aa, Artificial Sequence
  Ab No. 18 Heavy Chain (K1-18)_P17_PW amino acid sequence
VQLEESGDDLVKPGASVKLSCKASGHTFTSYWINWIKQRPGQGLEWIGRIAPGSGSTYYNEMFKGKATLTVDTSSSTAYIQLSSLSSEDSAVYFCARNLKYGNYGFAYWGQGTTVTVSS

SEQ ID NO: 85 - protein, 750 aa, Homo sapiens
  MISC_FEATURE (1)..(750): Human PSMA full length amino acid sequence (encoded by human PSMA mRNA, GenBank M99487.1) 750 amino acids
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 86 - protein, 372 aa, Homo sapiens
  MISC_FEATURE (1)..(372): Human PSMA fragment (amino acid positions 44 to 415) 372 amino acids
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRT

SEQ ID NO: 87 - DNA, 32 bp, Artificial Sequence
  positive strand primer
ccggaattcatgaaatcctccaatgaagctac

SEQ ID NO: 88 - DNA, 33 bp, Artificial Sequence
  negative strand primer
cccaagcttctcatgttcttctaggtctccacc

SEQ ID NO: 89 - DNA, 2653 bp, Homo sapiens
  misc_feature (1)..(2653): Human prostate-specific membrane antigen (PSM) mRNA, complete cds, GenBank M99487.1
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 90 - DNA, 12 bp, Artificial Sequence
  Linker sequence for positive strand primer
ccggaattcatg

SEQ ID NO: 91 - DNA, 13 bp, Artificial Sequence
  Linker sequence for negative strand primer
cccaagcttctca

SEQ ID NO: 92 - protein, 330 aa, Artificial Sequence
  HC human wild type IgG1 Fc region amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 990 bp, Artificial Sequence
  HC human wild type IgG1 Fc region amino acid sequence
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccttgcccagcaccagaactcctggggggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 94 - DNA, 321 bp, Artificial Sequence
  LC kappa constant domain nucleotide sequence
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaagacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 95 - protein, 107 aa, Artificial Sequence [sequence omitted]

SEQ ID NO: 96 - protein, 373 aa, Homo sapiens
  MISC_FEATURE (1)..(373): Human PSMA fragment (amino acid positions 44 to 415) with a methionine added at the N-terminus
MKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRT

SEQ ID NO: 97 - DNA, 297 bp, Artificial Sequence
  An exemplary SUMO tag nucleotide sequence
gctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggt

SEQ ID NO: 98 - protein, 99 aa, Artificial Sequence
  An exemplary SUMO tag amino acid sequence
ASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIG

SEQ ID NO: 99 - protein, 6 aa, Artificial sequence
  Minimal reactive peptide
MWNLLH

(19) United States
(12) Patent Application Publication
(10) Pub. No.: US 2024/0002530 A1
(43) Pub. Date: Jan. 4, 2024

(54) ANTI-PSMA ANTIBODIES AND METHODS OF USE

(71) Applicant: LXZ SERVICES LLC, Ardmore, PA (US)

(72) Inventor: Yong Liu, Bellevue, WA (US)

(21) Appl. No.: 18/251,828

(22) PCT Filed: Nov. 8, 2021
(86) PCT No.: PCT/US2021/058442

§ 371 Date: May 4, 2023

Related U.S. Application Data
(60) Provisional application No. 63/111,537, filed on Nov. 9, 2020.

(51) Int. Cl. C07K 16/30; C12N 15/63; A61P 35/00

(52) U.S. Cl. CPC C07K 16/3069; C12N 15/63; A61P 35/00; C07K 2317/14; C07K 2317/24; C07K 2317/41; C07K 2317/71; C07K 2317/565

(57) ABSTRACT

The present disclosure provides anti-PSMA antibodies or antigen binding fragments thereof, compositions comprising the antibodies or antigen binding fragments thereof, methods of treating cancer with the antibodies or antigen binding fragments thereof, recombinant PSMA polypeptides, and methods of using the recombinant PSMA polypeptides.

### BACKGROUND

Prostate-specific membrane antigen (PSMA), also known as glutamate carboxypeptidase II (GCPII), N-acetyl-L-aspartyl-L-glutamate peptidase I (NAALADase I), or NAAG peptidase, is a type-II cell glycoprotein that crosses the cell membrane with a molecular weight of about 100 kD. The PSMA gene is located on the short arm of chromosome 11, including 19 exons and 18 introns, and spans approximately 60 kb of the genomic DNA. The PSMA mRNA has a full length of 2252 bp, encoding 750 amino acids (aa). The short -NH2 terminus of the amino acid sequence of PSMA is located inside the cell membrane, and the long —COOH terminus is located outside the cell membrane. According to the position of the amino acid sequence of PSMA, PSMA can be divided into three parts: an intracellular part (amino acid positions 1-18), a transmembrane part (amino acid positions 19-43), and an extracellular region (amino acid positions 44-750). The extracellular part of PSMA contains three domains: a protease domain (amino acid positions 56-116, 352-591), an apical domain (amino acid positions 117-351), and a spiral domain (amino acid positions 592-750).

PSMA has two important enzyme activities: folate hydrolase and glutamate carboxypeptidase II. The receptor-like function of PSMA is related to the uptake of nutrients by tumor cells, and a large number of current studies have confirmed that PSMA plays an important role in the occurrence, growth, differentiation, and metastasis of tumor cells.

PSMA is a more sensitive and specific tumor marker for prostate cancer than prostate-specific antigen (PSA). PSMA is not only highly expressed in prostate cancer tissues, but can also be expressed in tumor neovascular endothelial cells in a variety of tumor tissues, such as kidney cancer, sarcoma, breast cancer, intestinal cancer, gastric cancer, and lung cancer. In recent years, it has been found that PSMA can be expressed in a variety of lung cancer tissues. Therefore, using PSMA as a target to prepare corresponding antibodies, especially monoclonal antibodies, is of great value for the diagnosis and treatment of prostate cancer and other cancers. To prepare monoclonal antibodies against PSMA, ideal B lymphocyte antigen epitopes of PSMA need to be determined first.

Some PSMA monoclonal antibodies have been successfully prepared currently, including 7E11-C5.3 targeting the intracellular epitopes of PSMA, and J591, J533, and J415 targeting the extracellular amino acids (aa) aa 490-aa 500 of PSMA. Except the 7E11-C5.3, which is a monoclonal antibody that specifically binds to the intracellular amino terminus, the others are all monoclonal antibodies that specifically bind to the extracellular hydroxyl terminus of PSMA. These monoclonal antibodies are considered to have clinical value in the diagnosis and treatment of prostate cancer. Some of these antibodies have undergone clinical studies. These antibodies, however, have clear deficiencies. First, although the PSMA protein consists of a glycoprotein, many research results have suggested that these antibodies do not recognize a glycopeptide as was initially thought, but recognize an intracellular epitope consisting of only the primary polypeptide chain. Second, the PSMA antigenic epitopes recognized by these antibodies are only small active peptides composed of a small number of amino acids. For example, the epitope recognized by the 7E11-C5.3 antibody is a minimal reactive peptide consisting of six amino acids (MWNLLH). Therefore, these antibodies have insufficient sensitivity and practicality in diagnosis and treatment.

### BRIEF SUMMARY

In one aspect, the present disclosure provides an isolated antibody, or an antigen-binding fragment thereof, wherein the antibody or antigen-binding fragment thereof comprises a heavy chain variable region (VH) and a light chain variable region (VL), wherein
(a) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:4, SEQ ID NO:5, and SEQ ID NO:6, respectively;
(b) the VH comprises a heavy chain CDR1 (VH-CDR1), heavy chain CDR2 (VH-CDR2), and heavy chain CDR3 (VH-CDR3) comprising the amino acid sequences of SEQ ID NO:7, SEQ ID NO:2, and SEQ ID NO:8, respectively; and the VL comprises a light chain CDR1 (VL-CDR1), light chain CDR2 (VL-CDR2), and light chain CDR3 (VL-CDR3) comprising the amino acid sequences of SEQ ID NO:9, SEQ ID NO:10, and SEQ ID NO:11, respectively;
(c) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:12, SEQ ID NO:13, and SEQ ID NO:14, respectively;
(d) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:15, SEQ ID NO:16, and SEQ ID NO:17, respectively;
(e) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:18, SEQ ID NO:19, and SEQ ID NO:20, respectively;
(f) the VH comprises a heavy chain CDR1 (VH-CDR1), heavy chain CDR2 (VH-CDR2), and heavy chain CDR3 (VH-CDR3) comprising the amino acid sequences of SEQ ID NO:21, SEQ ID NO:22, and SEQ ID NO:23, respectively; and the VL comprises a light chain CDR1 (VL-CDR1), light chain CDR2 (VL-CDR2), and light chain CDR3 (VL-CDR3) comprising the amino acid sequences of SEQ ID NO:24, SEQ ID NO:25, and SEQ ID NO:26, respectively;
(g) the VH comprises a heavy chain CDR1 (VH-CDR1), heavy chain CDR2 (VH-CDR2), and heavy chain CDR3 (VH-CDR3) comprising the amino acid sequences of SEQ ID NO:27, SEQ ID NO:28, and SEQ ID NO:29, respectively; and the VL comprises a light chain CDR1 (VL-CDR1), light chain CDR2 (VL-CDR2), and light chain CDR3 (VL-CDR3) comprising the amino acid sequences of SEQ ID NO:30, SEQ ID NO:31, and SEQ ID NO:32, respectively;
(h) the VH comprises a heavy chain CDR1 (VH-CDR1), heavy chain CDR2 (VH-CDR2), and heavy chain CDR3 (VH-CDR3) comprising the amino acid sequences of SEQ ID NO:33, SEQ ID NO:22, and SEQ ID NO:34, respectively; and the VL comprises a light chain CDR1 (VL-CDR1), light chain CDR2 (VL-CDR2), and light chain CDR3 (VL-CDR3) comprising the amino acid sequences of SEQ ID NO:35, SEQ ID NO:36, and SEQ ID NO:37, respectively;
(i) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:38, SEQ ID NO:39, and SEQ ID NO:40, respectively; or
(j) the VH comprises a heavy chain CDR1 (VH-CDR1), heavy chain CDR2 (VH-CDR2), and heavy chain CDR3 (VH-CDR3) comprising the amino acid sequences of SEQ ID NO:41, SEQ ID NO:28, and SEQ ID NO:29, respectively; and the VL comprises a light chain CDR1 (VL-CDR1), light chain CDR2 (VL-CDR2), and light chain CDR3 (VL-CDR3) comprising the amino acid sequences of SEQ ID NO:42, SEQ ID NO:43, and SEQ ID NO:44, respectively.

In another aspect, the present disclosure provides a method of treating cancer comprising administering to a patient in need thereof an effective amount of the antibody or antigen-binding fragment thereof as described herein.

In another aspect, the present disclosure provides recombinant PSMA polypeptides that do not include 418-567 of full-length PSMA.

Other objectives, advantages and novel features of the disclosure will become more apparent from the following detailed description.

### BRIEF DESCRIPTION OF THE SEVERAL VIEWS OF THE DRAWINGS

FIG. 1 shows a PSMA protease domain (extracellular functional structural domain) gene and a coding protein sequence (encoding the 44^{th} amino acid at the N-terminus to the 372^{nd} amino acid at the C-terminus of the PAMA protein) with a methionine added at the N-terminus.

FIG. 2 shows a flowchart of construction of the pET-28a-SUMO/PSMA plasmid.

FIG. 3 shows gel electrophoresis results of an RT-PCR on mRNA of a human prostate cancer tissue to obtain 1116-bp PSMA cDNA.

FIG. 4 shows a restriction endonuclease map of the pET-28a-SUMO plasmid and the sequence of a fragment that encodes His and small ubiquitin-like modifier (SUMO) tags.

FIG. 5 shows validation of double digestion of the pET-28a-SUMO vector. Lane “M” indicates the marker, and lanes “A-D” were four digested recombinant protein vectors.

FIG. 6 shows PCR identification results of the PSMA cDNA in the pET-28a-SUMO/PSMA plasmid. Lane “M” indicates the marker, and lanes “A-I” were the PCR products of the extracellular segment of the PSMA gene.

FIG. 7 shows a map of a prokaryotic recombinant protein expression vector, pET-28a-SUMO/PSMA plasmid that contains a polynucleotide sequence encoding the protease domain of human PSMA.

FIG. 8 shows sequence alignment between the PSMA cDNA in the pET-28a-SUMO/PSMA vector and the human PSMA mRNA gene sequence provided by GenBank: M99487.1.

FIG. 9 shows results of PSMA protein expression after induction of the pET-28a-SUMO/PSMA vector, in which the molecular weights of protein markers are 116 kD, 66.2 kD, 45 kD, 35 kD, 18.4 kD, and 14.4 kD from top to bottom sequentially. Lane “M” is the marker, lane “A” is protein expression from un-induced cells, and lane “B” is protein expression from induced cells.

FIG. 10 shows identification and purification of recombinant PSMA protein expression forms, in which the molecular weights of protein markers are 116 kD, 66.2 kD, 45 kD, 35 kD, 18.4 kD, and 14.4 kD from top to bottom sequentially. Lane “M” is the marker, lane “A” is from un-induced cells, lane “B” is total protein from induced cells, lane “C” is supernatant from induced cells, and lane “D” is precipitation from induced cells.

FIG. 11 shows recombinant protein expression results identified using a 6xHis monoclonal antibody (Mouse anti-6*His tag monoclonal antibody) by Western blotting. Lane “M” is the marker, lane “A” is from un-induced cells, lane “B” is total protein from induced cells, lane “C” is supernatant from induced cells, and lane “D” is precipitation from induced cells.

FIG. 12 shows electrophoresis of a renatured purified protein from inclusion bodies. Lane “M” is the marker, and lanes “A-D” were samples purified by nickel column.

FIG. 13 shows a method for preparing a monoclonal antibody (PSMA-McAb) containing the protease domain of human PSMA.

FIG. 14 shows the titers and subtypes of 18 clones of the PSMA-McAb obtained.

FIG. 15 shows results of binding of the PSMA-McAb derived from the 18 clones to prostate cancer cells identified by flow cytometry. The PSMA-McAb derived from the 18 clones each have undergone antigen-antibody reactions with prostate cancer LNCap cells and PC-3 cells.

FIG. 16 shows results of recognition of PSMA of prostate cancer cells by the PSMA-McAbs analyzed by Western blotting.

FIG. 17 shows results of recognition of PSMA of a prostate cancer tissue by the PSMA-McAb analyzed by immunohistochemistry.

FIG. 18 shows results of recognition of PSMA of a prostate cancer tissue by the PSMA-McAb analyzed by Immunofluorescence.

FIG. 19 shows inhibition of the proliferation of prostate cancer cells by the PSMA-McAbs.

FIG. 20 shows antibody-mediated complement-dependent cytotoxicity of the PSMA-McAbs.

### DETAILED DESCRIPTION

The present disclosure provides anti-PSMA antibodies or antigen-binding fragments thereof, which are useful in treating cancer, optionally in combination with other anti-cancer therapeutic agents.

The anti-PSMA antibodies disclosed herein have the advantages of recognizing epitopes of PSMA stable binding, high sensitivity, and high specificity. Additionally the anti-PSMA antibodies disclosed herein have the advantage of avoiding potential cross-reaction with the human transferrin receptor protein. The PSMA sequence has revealed that a portion of the coding region, from nucleotides (nt) 1513 to 1962 (GenBank: M99487.1, SEQ ID NO: 89), has 54% homology to human transferrin receptor mRNA. The corresponding amino acid sequence (aa) of PSMA is aa 418-aa 567. Thus, previously developed antibodies that bind to this region, such as J533 and other monoclonal antibodies against aa 490-aa 500 of PSMA, may cross-react with the human transferrin receptor protein, reducing the specificity of these antibodies. If these antibodies enter the human body for diagnosis and treatment, the safety of the antibodies will be reduced, which may cause potentially serious side effects.

In addition, since an important function of PSMA is its protease activity, monoclonal antibodies targeting epitopes in the protease domain (amino acid 56 to amino acid 116, and amino acid 352 to amino acid 591) of PSMA, such as the antibodies disclosed herein, may have important medical diagnostic and therapeutic value.

Prior to setting forth this disclosure in more detail, it may be helpful to an understanding thereof to provide definitions of certain terms used herein. Additional definitions are set forth throughout this disclosure.

In the present description, any range such as concentration range, percentage range, ratio range, and integer range is to be understood to include all values and subranges with the range, unless otherwise indicated.

As used herein, the term “about” means ±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

As used herein, “PSMA” or “prostate-specific membrane antigen” or “glutamate carboxypeptidase II” or “GCPII” or “N-acetyl-L-aspartyl-L-glutamate peptidase I” or “NAALADase I,” or “NAAG peptidase” refers to an enzyme that in humans is encoded by the FOLH1 (folate hydrolase 1) gene (GenBank Accession No. M99487.1, SEQ ID NO:89) that can have aberrant overexpression in a variety of cancer types such as in prostate cancer tissues, and tumor neovascular endothelial cells in a variety of tumor tissues, such as kidney cancer, sarcoma, breast cancer, intestinal cancer, gastric cancer, and lung cancer. In some embodiments, PSMA is a human PSMA as set forth in SEQ ID NO:85.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

As used herein, “protein” or “polypeptide” as used herein refers to a compound made up of amino acid residues that are covalently linked by peptide bonds. The term “protein” may be synonymous with the term “polypeptide” or may refer, in addition, to a complex of two or more polypeptides. A polypeptide may further contain other components (e.g., covalently bound), such as a tag, a label, a bioactive molecule, or any combination thereof. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

As described herein, a “variant” polypeptide species has one or more non-natural amino acids, one or more amino acid substitutions, one or more amino acid insertions, one or more amino acid deletions, or any combination thereof at one or more sites relative to a reference polypeptide as presented herein. In certain embodiments, “variant” means a polypeptide having a substantially similar activity (e.g., enzymatic function, immunogenicity) or structure relative to a reference polypeptide. A variant of a reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference polypeptide as determined by sequence alignment programs and parameters known in the art. The variant can result from, for example, a genetic polymorphism or human manipulation. Conservative substitutions of amino acids are well known and may occur naturally or may be introduced when a protein is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, NY, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare polypeptide variants (see, e.g., Sambrook et al., supra).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. The algorithm used herein for determining percent sequence identity and sequence similarity is the BLAST 2.0 algorithm, as described in Altschul et al. “Gapped BLAST and PSI-BLAST: a new generation of protein database search programs,” Nucleic Acids Res. 2007, 25, 3389-3402, with the parameters set to default values.

As used herein, a “fusion protein” comprises a single chain polypeptide having at least two distinct domains, wherein the domains are not naturally found together in a protein. A nucleic acid molecule encoding a fusion protein may be constructed using PCR, recombinantly engineered, or the like, or such fusion proteins can be made synthetically. A fusion protein may further contain other components (e.g., covalently bound), such as a tag, linker, or bioactive molecule.

A “nucleic acid molecule” or “polynucleotide” refers to a polymeric compound containing nucleotides that are covalently linked by 3′-5′ phosphodiester bonds. Nucleic acid molecules include polyribonucleic acid (RNA), polydeoxyribonucleic acid (DNA), which includes genomic DNA, mitochondrial DNA, cDNA, or vector DNA. A nucleic acid molecule may be double stranded or single stranded, and if single stranded, may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of the polynucleotides of this disclosure are also contemplated. Variant polynucleotides are at least 80%, 85%, 90%, 95%, 99%, or 99.9% identical to a reference polynucleotide as described herein, or that hybridizes to a reference polynucleotide of defined sequence under stringent hybridization conditions of 0.015M sodium chloride, 0.0015M sodium citrate at about 65°-68° C. or 0.015M sodium chloride, sodium citrate, and 50% formamide at about 42° C. The polynucleotide variants retain the capacity to encode an immunoglobulin-like binding protein or antigen-binding fragment thereof having the functionality described herein.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the co-existing materials in the natural system, is isolated. Such polynucleotide could be part of a vector and/or such polynucleotide or polypeptide could be part of a composition (e.g., a cell lysate), and still be isolated in that such vector or composition is not part of the natural environment for the nucleic acid or polypeptide.

As used herein, a polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein, or other activity encoded by an exogenous nucleic acid molecule introduced into the host cell, even if the host cell encodes a homologous protein or activity.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

“Expression cassette” refers a polynucleotide encoding a polypeptide of interest operably linked to at least one polynucleotide encoding an expression control sequence. The expression cassette can include in the 5′-3′ direction of transcription, a transcriptional and translational initiation region (i.e., a promoter), polynucleotide encoding a polypeptide of interest or active variant or fragment thereof, and a transcriptional and translational termination region (i.e., termination region). The regulatory regions (i.e., promoters, transcriptional regulatory regions, and translational termination regions) and/or the polynucleotide or active variant or fragment thereof may be native/analogous to the host cell or to each other. Alternatively, the regulatory regions and/or the polynucleotide of or active variant or fragment thereof may be heterologous to the host cell or to each other.

“Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or noncontiguous. When used to refer to the joining of two protein coding regions, by operably linked is intended that the coding regions are in the same reading frame. The cassette may additionally contain at least one additional coding sequence/gene to be co-transformed into the organism. Alternatively, the additional coding sequences/gene(s) can be provided on multiple expression cassettes. Such an expression cassette is provided with a plurality of restriction sites and/or recombination sites for insertion of a coding polynucleotide of interest or active variant or fragment thereof to be under the transcriptional regulation of the regulatory regions (e.g., promoter). The expression cassette may additionally contain selectable marker genes.

“Expression control sequence” refers to a segment of a nucleic acid molecule which is capable of increasing or decreasing the expression of a polypeptide encoded by the expression cassette. Examples of expression control regions include promoters, transcriptional regulatory regions, and translational termination regions.

As used herein, the term “expression” refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

As described herein, more than one heterologous nucleic acid molecule can be introduced into a host cell as separate nucleic acid molecules, as a plurality of individually controlled genes, as a polycistronic nucleic acid molecule (e.g., a heavy chain and a light chain of an antibody), as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

Additional definitions are provided in the sections below.

### Anti-PSMA Antibodies or Antigen-Binding Fragments Thereof

In one aspect, antibodies (e.g., isolated monoclonal antibodies) or antigen-binding fragments thereof that specifically bind to PSMA, also referred to as anti-PSMA antibodies or antigen-binding fragments thereof, are provided.

In some embodiments, antibodies or antigen-binding fragments thereof of the present disclosure specifically bind to PSMA (such as to the PSMA protease domain) with high affinity. As used herein, “specifically binds” or “specific for” refer to an association or union of a binding protein (e.g., an anti-PSMA antibody) or a binding domain (or fusion protein thereof) to a target molecule (e.g., PSMA) with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{D}) of a particular binding interaction with units of M (e.g., 10⁻⁵ M to 10⁻¹³ M) (which equals the ratio of the off-rate [k_{off}] to the on-rate [kₒₙ] for this association reaction).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

A monoclonal antibody or antigen-binding portion thereof may be non-human, chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

The terms “VL” and “VH” refer to the variable binding regions from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be determined according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Mo. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. CDRs of the anti-PSMA antibodies provided in the present disclosure are identified according to the IMGT numbering scheme unless indicated otherwise.

In some embodiments, an isolated antibody or an antigen-binding fragment thereof that binds to PSMA is provided, wherein the antibody or antigen-binding fragment thereof comprises a heavy chain variable region (VH) and a light chain variable region (VL), wherein:
(a) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:4, SEQ ID NO:5, and SEQ ID NO:6, respectively;
(b) the VH comprises a heavy chain CDR1 (VH-CDR1), heavy chain CDR2 (VH-CDR2), and heavy chain CDR3 (VH-CDR3) comprising the amino acid sequences of SEQ ID NO:7, SEQ ID NO:2, and SEQ ID NO:8, respectively; and the VL comprises a light chain CDR1 (VL-CDR1), light chain CDR2 (VL-CDR2), and light chain CDR3 (VL-CDR3) comprising the amino acid sequences of SEQ ID NO:9, SEQ ID NO:10, and SEQ ID NO:11, respectively;
(c) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:12, SEQ ID NO:13, and SEQ ID NO:14, respectively;
(d) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:15, SEQ ID NO:16, and SEQ ID NO:17, respectively;
(e) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:18, SEQ ID NO:19, and SEQ ID NO:20, respectively;
(f) the VH comprises a heavy chain CDR1 (VH-CDR1), heavy chain CDR2 (VH-CDR2), and heavy chain CDR3 (VH-CDR3) comprising the amino acid sequences of SEQ ID NO:21, SEQ ID NO:22, and SEQ ID NO:23, respectively; and the VL comprises a light chain CDR1 (VL-CDR1), light chain CDR2 (VL-CDR2), and light chain CDR3 (VL-CDR3) comprising the amino acid sequences of SEQ ID NO:24, SEQ ID NO:25, and SEQ ID NO:26, respectively;
(g) the VH comprises a heavy chain CDR1 (VH-CDR1), heavy chain CDR2 (VH-CDR2), and heavy chain CDR3 (VH-CDR3) comprising the amino acid sequences of SEQ ID NO:27, SEQ ID NO:28, and SEQ ID NO:29, respectively; and the VL comprises a light chain CDR1 (VL-CDR1), light chain CDR2 (VL-CDR2), and light chain CDR3 (VL-CDR3) comprising the amino acid sequences of SEQ ID NO:30, SEQ ID NO:31, and SEQ ID NO:32, respectively;
(h) the VH comprises a heavy chain CDR1 (VH-CDR1), heavy chain CDR2 (VH-CDR2), and heavy chain CDR3 (VH-CDR3) comprising the amino acid sequences of SEQ ID NO:33, SEQ ID NO:22, and SEQ ID NO:34, respectively; and the VL comprises a light chain CDR1 (VL-CDR1), light chain CDR2 (VL-CDR2), and light chain CDR3 (VL-CDR3) comprising the amino acid sequences of SEQ ID NO:35, SEQ ID NO:36, and SEQ ID NO:37, respectively;
(i) the VH comprises a heavy chain CDR1 (VH-CDR1), heavy chain CDR2 (VH-CDR2), and heavy chain CDR3 (VH-CDR3) comprising the amino acid sequences of SEQ ID NO:1, SEQ ID NO:2, and SEQ ID NO:3, respectively; and the VL comprises a light chain CDR1 (VL-CDR1), light chain CDR2 (VL-CDR2), and light chain CDR3 (VL-CDR3) comprising the amino acid sequences of SEQ ID NO:38, SEQ ID NO:39, and SEQ ID NO:40, respectively; or
(j) the VH comprises a heavy chain CDR1 (VH-CDR1), heavy chain

CDR2 (VH-CDR2), and heavy chain CDR3 (VH-CDR3) comprising the amino acid sequences of SEQ ID NO:41, SEQ ID NO:28, and SEQ ID NO:29, respectively; and the VL comprises a light chain CDR1 (VL-CDR1), light chain CDR2 (VL-CDR2), and light chain CDR3 (VL-CDR3) comprising the amino acid sequences of SEQ ID NO:42, SEQ ID NO:43, and SEQ ID NO:44, respectively.

In some embodiments, an isolated antibody or an antigen-binding fragment thereof that binds to PSMA is provided, wherein:
(a) the VH comprises an amino acid sequence that has at least 90%, 91%, 92%, 93%, 94%, 95%, 96%, 97%, 98%, or 99% identity with the amino acid sequence of SEQ ID NO:48 and the VL comprises an amino acid sequence that has at least 90%, 91%, 92%, 93%, 94%, 95%, 96%, 97%, 98%, or 99% identity with the amino acid sequence of SEQ ID NO:46;
(b) the VH comprises an amino acid sequence that has at least 90%, 91%, 92%, 93%, 94%, 95%, 96%, 97%, 98%, or 99% identity with the amino acid sequence of SEQ ID NO:52, and the VL comprises an amino acid sequence that has at least 90%, 91%, 92%, 93%, 94%, 95%, 96%, 97%, 98%, or 99% identity with the amino acid sequence of SEQ ID NO:50;
(c) the VH comprises an amino acid sequence that has at least 90%, 91%, 92%, 93%, 94%, 95%, 96%, 97%, 98%, or 99% identity with the amino acid sequence of SEQ ID NO:56, and the VL comprises an amino acid sequence that has at least 90%, 91%, 92%, 93%, 94%, 95%, 96%, 97%, 98%, or 99% identity with the amino acid sequence of SEQ ID NO:54;

(d) the VH comprises an amino acid sequence that has at least 90%, 91%, 92%, 93%, 94%, 95%, 96%, 97%, 98%, or 99% identity with the amino acid sequence of SEQ ID NO:60, and the VL comprises an amino acid sequence that has at least 90%, 91%, 92%, 93%, 94%, 95%, 96%, 97%, 98%, or 99% identity with the amino acid sequence of SEQ ID NO:58;
(e) the VH comprises an amino acid sequence that has at least 90%, 91%, 92%, 93%, 94%, 95%, 96%, 97%, 98%, or 99% identity with the amino acid sequence of SEQ ID NO:64, and the VL comprises an amino acid sequence that has at least 90%, 91%, 92%, 93%, 94%, 95%, 96%, 97%, 98%, or 99% identity with the amino acid sequence of SEQ ID NO:62;
(f) the VH comprises an amino acid sequence that has at least 90%, 91%, 92%, 93%, 94%, 95%, 96%, 97%, 98%, or 99% identity with the amino acid sequence of SEQ ID NO:68, and the VL comprises an amino acid sequence that has at least 90%, 91%, 92%, 93%, 94%, 95%, 96%, 97%, 98%, or 99% identity with the amino acid sequence of SEQ ID NO:66;
(g) the VH comprises an amino acid sequence that has at least 90%, 91%, 92%, 93%, 94%, 95%, 96%, 97%, 98%, or 99% identity with the amino acid sequence of SEQ ID NO:72, and the VL comprises an amino acid sequence that has at least 90%, 91%, 92%, 93%, 94%, 95%, 96%, 97%, 98%, or 99% identity with the amino acid sequence of SEQ ID NO:70;
(h) the VH comprises an amino acid sequence that has at least 90%, 91%, 92%, 93%, 94%, 95%, 96%, 97%, 98%, or 99% identity with the amino acid sequence of SEQ ID NO:76, and the VL comprises an amino acid sequence that has at least 90%, 91%, 92%, 93%, 94%, 95%, 96%, 97%, 98%, or 99% identity with the amino acid sequence of SEQ ID NO:74;
(i) the VH comprises an amino acid sequence that has at least 90%, 91%, 92%, 93%, 94%, 95%, 96%, 97%, 98%, or 99% identity with the amino acid sequence of SEQ ID NO:80, and the VL comprises an amino acid sequence that has at least 90%, 91%, 92%, 93%, 94%, 95%, 96%, 97%, 98%, or 99% identity with the amino acid sequence of SEQ ID NO:78; or
(j) the VH comprises an amino acid sequence that has at least 90%, 91%, 92%, 93%, 94%, 95%, 96%, 97%, 98%, or 99% identity with the amino acid sequence of SEQ ID NO:84, and the VL comprises an amino acid sequence that has at least 90%, 91%, 92%, 93%, 94%, 95%, 96%, 97%, 98%, or 99% identity with the amino acid sequence of SEQ ID NO:82.

In some embodiments, an isolated antibody or an antigen-binding fragment thereof that binds to PSMA is provided, wherein:
(a) the VH comprises an amino acid sequence that has at least 90%, 91%, 92%, 93%, 94%, 95%, 96%, 97%, 98%, or 99% identity with the amino acid sequence of SEQ ID NO:48, and the VL comprises an amino acid sequence that has at least 90%, 91%, 92%, 93%, 94%, 95%, 96%, 97%, 98%, or 99% identity with the amino acid sequence of SEQ ID NO:46, provided that the amino acid sequences of the VH-CDRs (SEQ ID NOS:1-3) and VL-CDRs (SEQ ID NOS:4-6) are unchanged;
(b) the VH comprises an amino acid sequence that has at least 90%, 91%, 92%, 93%, 94%, 95%, 96%, 97%, 98%, or 99% identity with the amino acid sequence of SEQ ID NO:52, and the VL comprises an amino acid sequence that has at least 90%, 91%, 92%, 93%, 94%, 95%, 96%, 97%, 98%, or 99% identity with the amino acid sequence of SEQ ID NO:50, provided that the amino acid sequences of the VH-CDRs (SEQ ID NOS:7, 2, and 8) and VL-CDRs (SEQ ID NOS:9-11) are unchanged;
(c) the VH comprises an amino acid sequence that has at least 90%, 91%, 92%, 93%, 94%, 95%, 96%, 97%, 98%, or 99% identity with the amino acid sequence of SEQ ID NO:56, and the VL comprises an amino acid sequence that has at least 90%, 91%, 92%, 93%, 94%, 95%, 96%, 97%, 98%, or 99% identity with the amino acid sequence of SEQ ID NO:54, provided that the amino acid sequences of the VH-CDRs (SEQ ID NOS:1-3) and VL-CDRs (SEQ ID NOS:12-14) are unchanged;
(d) the VH comprises an amino acid sequence that has at least 90%, 91%, 92%, 93%, 94%, 95%, 96%, 97%, 98%, or 99% identity with the amino acid sequence of SEQ ID NO:60, and the VL comprises an amino acid sequence that has at least 90%, 91%, 92%, 93%, 94%, 95%, 96%, 97%, 98%, or 99% identity with the amino acid sequence of SEQ ID NO:58, provided that the amino acid sequences of the VH-CDRs (SEQ ID NOS:1-3) and VL-CDRs (SEQ ID NOS:15-17) are unchanged;
(e) the VH comprises an amino acid sequence that has at least 90%, 91%, 92%, 93%, 94%, 95%, 96%, 97%, 98%, or 99% identity with the amino acid sequence of SEQ ID NO:64, and the VL comprises an amino acid sequence that has at least 90%, 91%, 92%, 93%, 94%, 95%, 96%, 97%, 98%, or 99% identity with the amino acid sequence of SEQ ID NO:62, provided that the amino acid sequences of the VH-CDRs (SEQ ID NOS:1-3) and VL-CDRs (SEQ ID NOS:18-20) are unchanged;
(f) the VH comprises an amino acid sequence that has at least 90%, 91%, 92%, 93%, 94%, 95%, 96%, 97%, 98%, or 99% identity with the amino acid sequence of SEQ ID NO:68, and the VL comprises an amino acid sequence that has at least 90%, 91%, 92%, 93%, 94%, 95%, 96%, 97%, 98%, or 99% identity with the amino acid sequence of SEQ ID NO:66, provided that the amino acid sequences of the VH-CDRs (SEQ ID NOS:21-23) and VL-CDRs (SEQ ID NOS:24-26) are unchanged;
(g) the VH comprises an amino acid sequence that has at least 90%, 91%, 92%, 93%, 94%, 95%, 96%, 97%, 98%, or 99% identity with the amino acid sequence of SEQ ID NO:72, and the VL comprises an amino acid sequence that has at least 90%, 91%, 92%, 93%, 94%, 95%, 96%, 97%, 98%, or 99% identity with the amino acid sequence of SEQ ID NO:70, provided that the amino acid sequences of the VH-CDRs (SEQ ID NOS:27-29) and VL-CDRs (SEQ ID NOS:30-32) are unchanged;
(h) the VH comprises an amino acid sequence that has at least 90%, 91%, 92%, 93%, 94%, 95%, 96%, 97%, 98%, or 99% identity with the amino acid sequence of SEQ ID NO:76, and the VL comprises an amino acid sequence that has at least 90%, 91%, 92%, 93%, 94%, 95%, 96%, 97%, 98%, or 99% identity with the amino acid sequence of SEQ ID NO:74, provided that the amino acid sequences of the VH-CDRs (SEQ ID NOS:33, 22, 34) and VL-CDRs (SEQ ID NOS:35-37) are unchanged;
(i) the VH comprises an amino acid sequence that has at least 90%, 91%, 92%, 93%, 94%, 95%, 96%, 97%, 98%, or 99% identity with the amino acid sequence of SEQ ID NO:80, and the VL comprises an amino acid sequence that has at least 90%, 91%, 92%, 93%, 94%, 95%, 96%, 97%, 98%, or 99% identity with the amino acid sequence of SEQ ID NO:78, provided that the amino acid sequences of the VH-CDRs (SEQ ID NOS:1-3) and VL-CDRs (SEQ ID NOS:38-40) are unchanged; or
(j) the VH comprises an amino acid sequence that has at least 90%, 91%, 92%, 93%, 94%, 95%, 96%, 97%, 98%, or 99% identity with the amino acid sequence of SEQ ID NO:84, and the VL comprises an amino acid sequence that has at least 90%, 91%, 92%, 93%, 94%, 95%, 96%, 97%, 98%, or 99% identity with the amino acid sequence of SEQ ID NO:82, provided that the amino acid sequences of the VH-CDRs (SEQ ID NOS:41, 28, 29) and VL-CDRs (SEQ ID NOS:42, 43, 44) are unchanged.

In some embodiments, an isolated antibody or an antigen-binding fragment thereof that binds to PSMA is provided, wherein:
(a) the VH comprises the amino acid sequence of SEQ ID NO:48 and the VL comprises the amino acid sequence of SEQ ID NO:46;
(b) the VH comprises the amino acid sequence of SEQ ID NO:52, and the VL comprises the amino acid sequence of SEQ ID NO:50;
(c) the VH comprises the amino acid sequence of SEQ ID NO:56, and the VL comprises the amino acid sequence of SEQ ID NO:54;
(d) the VH comprises the amino acid sequence of SEQ ID NO:60, and the VL comprises the amino acid sequence of SEQ ID NO:58;
(e) the VH comprises the amino acid sequence of SEQ ID NO:64, and the VL comprises the amino acid sequence of SEQ ID NO:62;
(f) the VH comprises the amino acid sequence of SEQ ID NO:68, and the VL comprises the amino acid sequence of SEQ ID NO:66;
(g) the VH comprises the amino acid sequence of SEQ ID NO:72, and the VL comprises the amino acid sequence of SEQ ID NO:70;
(h) the VH comprises the amino acid sequence of SEQ ID NO:76, and the VL comprises the amino acid sequence of SEQ ID NO:74;
(i) the VH comprises the amino acid sequence of SEQ ID NO:80, and the VL comprises the amino acid sequence of SEQ ID NO:78; or
(j) the VH comprises the amino acid sequence of SEQ ID NO:84, and the VL comprises the amino acid sequence of SEQ ID NO:82.

In some embodiments, an anti-PSMA antibody of the present disclosure comprises a heavy chain (HC) and a light chain (LC). The heavy chain typically comprises a VH and a heavy chain constant region (CH). Depending on the antibody isotype from which it derives, a heavy chain constant region may comprise CH1, CH2, and CH3 domains (IgG). In some embodiments, the heavy chain constant region comprises a human IgG1, IgG2, IgG3, or IgG4 constant region. An exemplary human IgG1 heavy chain constant region amino acid sequence comprises an amino acid sequence of SEQ ID NO:92. Another exemplary human IgG1 heavy chain constant region amino acid sequence comprises an amino acid sequence of SEQ ID NO: 93.

The light chain typically comprises a VL and a light chain constant region (CL). In some embodiments, a CL comprises a C kappa (“CK”) constant region. In some embodiments, a CL comprises a C lambda (Cλ) constant region. An exemplary human light chain C kappa constant region nucleic acid sequence comprises a nucleic acid sequence of SEQ ID NO:94. An exemplary human light chain C lambda constant region amino acid sequence comprises an amino acid sequence of SEQ ID NO:95. In some embodiments, an anti-PSMA antibody of the present disclosure comprises two heavy chains and two light chains, covalently connected by disulfide bridges.

In some embodiments, the anti-PSMA antibody comprises an IgG1 constant region comprising an amino acid sequence having 90%, 91%, 92%, 93%, 94%, 95%, 96%, 97%, 98%, 99%, or 100% identity to the amino acid sequence of SEQ ID NO:92. In some embodiments, an antibody or antigen-binding fragment of the present disclosure comprises an IgG1 constant region comprising an amino acid sequence having 90%, 91%, 92%, 93%, 94%, 95%, 96%, 975, 98%, 99%, or 100% identity to the amino acid sequence of SEQ ID NO:93.

In some embodiments, the antibody or antigen-binding fragment of the present disclosure comprises an Fc region portion. As used herein, “Fc region portion” refers to the heavy chain constant region segment of the Fc fragment (the “fragment crystallizable” region or Fc region) from an antibody, which can include one or more constant domains, such as CH2, CH3, or both. In some embodiments, an Fc region portion includes the CH2 and CH3 domains of an IgG antibody. In some embodiments, a CH2CH3 structure has sub-region domains from the same antibody isotype and are human, such as human IgG1, IgG2, IgG3, or IgG4 (e.g., CH2CH3 from human IgG1). By way of background, an Fc region is responsible for the effector functions of an antibody, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al. Nature 337: 525, 1989). In some embodiments, a Fc region portion in an antibody or antigen-binding fragment of the present disclosure will be capable of mediating one or more of these effector functions. In some embodiments, a Fc region portion in an antibody or antigen-binding fragment of the present disclosure has normal effector function, meaning having less than 25%, 20%, 15%, 10%, 5%, 1% difference in effector function (e.g., ADCC, CDC, or both) as compared to a wild type IgG1 antibody.

In some embodiments, an Fc region portion in an antibody or antigen-binding fragment of the present disclosure has a reduction in one or more of these effector functions or lack one or more effector functions by way of, for example, one or more amino acid substitutions or deletions in the Fc region portion known in the art. An antibody or antigen-binding fragment having a mutated or variant Fc region portion having reduced effector function means that the antibody exhibits a decrease of at least 20%, 25%, 30%, 35%, 40%, 45%, 50%, 55%, 60%, 65%, 70%, 75%, 80%, 85%, 90%, 95%, or 99% in FcR binding, ADCC, CDC, or any combination thereof, as compared to an antibody having a wild type Fc region portion. In some embodiments, the mutated or variant Fc region portion exhibits decreased binding to FcγRI (CD64), FcγRIIA (CD32), FcγRIIIA (CD16a), FcγRIIIB (CD16b), or any combination thereof. In some embodiments, the Fc region portion in an antibody or antigen-binding fragment of the present disclosure is a variant Fc region portion having reduced ADCC, CDC, or both. In some embodiments, the Fc region portion is a variant IgG1 Fc region portion comprising a mutation corresponding to amino acid E233P, L234V, L234A, L235A, L235E, ΔG236, G237A, E318A, K320A, K322A, A327G, P329G, A330S, P331S, or any combination thereof, as numbered according to the EU set forth in Kabat. For example, amino acid substitutions L234A, L235E, G237A introduced into an IgG1 Fc region portion reduces binding to FcγRI, FcγRIIa, and FcγRIII receptors, and A330S and P331S introduced into an IgG1 Fc region portion reduces C1q-mediated complement fixation. In some embodiments, the Fc region portion is a variant IgG1 Fc region portion comprising mutations corresponding to E233P, L234V, L235A, ΔG236, A327G, A330S, and P331S, as numbered according to the EU set forth in Kabat.

In some embodiments, the antibody or antigen-binding fragment thereof of the present disclosure is glycosylated. IgG subtype antibodies contain a conserved glycosylation site at amino acid N297 in the CH2 domain of the Fc region portion. In some such embodiments, the Fc region portion in an antibody or antigen-binding fragment of the present disclosure comprises a N297 as numbered according to EU set forth in Kabat. In some embodiments, the antibody or antigen-binding fragment of the present disclosure comprises a mutation that alters glycosylation at N297 in the Fc region portion, optionally wherein the mutation that alters glycosylation comprises N297A, N297Q, or N297G. In some embodiments, an antibody or antigen-binding fragment thereof comprising a N297A, N297Q, or N297G mutation exhibits reduced Fc interaction with one or more low affinity FcγR(s), reduced CDC, reduced ADCC, or any combination thereof.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, an Fc polypeptide of the present disclosure comprises two CH2-CH3 polypeptides that associate to form a dimer.

As used herein, unless otherwise provided, a position of an amino acid residue in the constant region of human IgG1 heavy chain is numbered assuming that the variable region of human IgG1 is composed of 128 amino acid residues according to the Kabat numbering convention. The numbered constant region of human IgG1 heavy chain is then used as a reference for numbering amino acid residues in constant regions of other immunoglobulin heavy chains. A position of an amino acid residue of interest in a constant region of an immunoglobulin heavy chain other than human IgG1 heavy chain is the position of the amino acid residue in human IgG1 heavy chain with which the amino acid residue of interest aligns. Alignments between constant regions of human IgG1 heavy chain and other immunoglobulin heavy chains may be performed using software programs known in the art, such as the Megalign program (DNASTAR Inc.) using the Clustal W method with default parameters. According to the numbering system described herein, for example, although human IgG2 CH2 region may have an amino acid deletion near its amino-terminus compared with other CH2 regions, the position of the “N” located at 296 in human IgG2 CH2 is still considered position 297 because this residue aligns with “N” at position 297 in human IgG1 CH2.

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

In some embodiments, the anti-PSMA antibody or antigen binding fragment thereof of the present disclosure is chimeric, humanized, or human.

In some embodiments, the anti-PSMA antibody or antigen-binding fragment thereof binds to the extracellular protease domain of PSMA. The extracellular region of PSMA includes three domains: a protease domain, an apical domain, and the C-terminal domain. The protease domain includes a first region (amino acid 56 to amino acid 116 of full-length human PSMA) and a second region (amino acid 352 to amino acid 591 of full-length human PSMA), which are separated by the apical domain.

In some embodiments, the anti-PSMA antibody or antigen-binding fragment thereof binds to PSMA expressed on the surface of cancer cells. Examples of types of cancer cells that express PSMA include prostate cancer, lung cancer, sarcoma, breast cancer, kidney cancer, and digestive tract malignant tumor cells. In some embodiments, the anti-PSMA antibody or antigen-binding fragment thereof induces antibody-mediated complement-dependent killing of cancer cells that express PSMA.

### Nucleic Acids, Vectors, and Host Cells

In another aspect, the present disclosure provides one or more isolated polynucleotide that encodes the anti-PSMA antibody or antigen binding fragment thereof as described herein. In some embodiments, the isolated polynucleotide encodes the VH, the VL, or both the VH and VL of the antibody or antigen binding fragment thereof. In some embodiments, the isolated polynucleotide encodes the heavy chain, the light chain, or both the heavy and light chain of the antibody or antigen binding fragment thereof. In some embodiments, the polynucleotide encoding the anti-PSMA antibody or antigen binding fragment thereof is codon optimized to enhance or maximize expression in certain types of cells (e.g., Scholten et al., Clin. Immunol. 119: 135-145, 2006). As used herein a “codon optimized” polynucleotide is a heterologous polypeptide having codons modified with silent mutations corresponding to the abundances of host cell tRNA levels.

In some embodiments, the present disclosure provides a set of isolated polynucleotides comprising:
(a) a polynucleotide sequence encoding a polypeptide having an amino acid sequence of SEQ ID NO: 48 and a polynucleotide sequence encoding a polypeptide having an amino acid sequence of SEQ ID NO: 46;
(b) a polynucleotide sequence encoding a polypeptide having an amino acid sequence of SEQ ID NO: 52 and a polynucleotide sequence encoding a polypeptide having an amino acid sequence of SEQ ID NO: 50;
(c) a polynucleotide sequence encoding a polypeptide having an amino acid sequence of SEQ ID NO: 56 and a polynucleotide sequence encoding a polypeptide having an amino acid sequence of SEQ ID NO: 54;
(d) a polynucleotide sequence encoding a polypeptide having an amino acid sequence of SEQ ID NO: 60and a polynucleotide sequence encoding a polypeptide having an amino acid sequence of SEQ ID NO: 58;
(e) a polynucleotide sequence encoding a polypeptide having an amino acid sequence of SEQ ID NO: 64 and a polynucleotide sequence encoding a polypeptide having an amino acid sequence of SEQ ID NO: 62;
(f) a polynucleotide sequence encoding a polypeptide having an amino acid sequence of SEQ ID NO: 68 and a polynucleotide sequence encoding a polypeptide having an amino acid sequence of SEQ ID NO: 66;
(g) a polynucleotide sequence encoding a polypeptide having an amino acid sequence of SEQ ID NO: 72 and a polynucleotide sequence encoding a polypeptide having an amino acid sequence of SEQ ID NO:70;
(h) a polynucleotide sequence encoding a polypeptide having an amino acid sequence of SEQ ID NO: 76 and a polynucleotide sequence encoding a polypeptide having an amino acid sequence of SEQ ID NO: 74;
(i) a polynucleotide sequence encoding a polypeptide having an amino acid sequence of SEQ ID NO: 80 and a polynucleotide sequence encoding a polypeptide having an amino acid sequence of SEQ ID NO: 78; or
(j) a polynucleotide sequence encoding a polypeptide having an amino acid sequence of SEQ ID NO: 84 and a polynucleotide sequence encoding a polypeptide having an amino acid sequence of SEQ ID NO: 82.

In some embodiments, the set of isolated polynucleotides comprises:
(a) a polynucleotide sequence of SEQ ID NO: 47 and a polynucleotide sequence of SEQ ID NO: 45;
(b) a polynucleotide sequence of SEQ ID NO: 51 and a polynucleotide sequence of SEQ ID NO: 49;
(c) a polynucleotide sequence of SEQ ID NO: 55 and a polynucleotide sequence of SEQ ID NO: 53;
(d) a polynucleotide sequence of SEQ ID NO: 59 and a polynucleotide sequence of SEQ ID NO: 57;
(e) a polynucleotide sequence of SEQ ID NO: 63 and a polynucleotide sequence of SEQ ID NO: 61;
(f) a polynucleotide sequence of SEQ ID NO: 67 and a polynucleotide sequence SEQ ID NO: 65;
(g) a polynucleotide of SEQ ID NO: 71 and a polynucleotide sequence of SEQ ID NO:69;
(h) a polynucleotide sequence of SEQ ID NO: 75 and a polynucleotide sequence of SEQ ID NO: 73;
(i) a polynucleotide sequence of SEQ ID NO: 79 and a polynucleotide sequence of SEQ ID NO: 77; or
(j) a polynucleotide sequence of SEQ ID NO: 83 and a polynucleotide sequence of SEQ ID NO: 81.

In some embodiments, a polynucleotide molecule encoding an anti-PSMA antibody or antigen binding fragment thereof of the present disclosure (e.g., an antibody heavy chain and light chain, or VH and VL regions) comprises a nucleic acid sequence for a heavy chain or VH region and a light chain or VL, respectively, wherein the heavy chain or VH region is separated from the light chain or VL region by a 2A self-cleaving peptide. In some embodiments, the 2A self-cleaving peptide is a porcine teschovirus-1 (P2A), equine rhinitis A virus (E2A), Thosea asigna virus (T2A), foot-and-mouth disease virus (F2A), or any combination thereof (see, e.g., Kim et al., PLOS One 6:e18556, 2011, which 2A nucleic acid and amino acid sequences are incorporated herein by reference in their entirety).

In another aspect, an expression construct comprising a nucleic acid encoding an anti-PSMA antibody or antigen binding fragment thereof as described herein is provided. In some embodiments, a nucleic acid may be operably linked to an expression control sequence (e.g., expression construct). As used herein, “expression construct” refers to a DNA construct containing a nucleic acid molecule that is operably-linked to a suitable control sequence capable of effecting the expression of the nucleic acid molecule in a suitable host. An expression construct may be present in a vector (e.g., a bacterial vector, a viral vector) or may be integrated into a genome. The term “operably linked” refers to the association of two or more nucleic acids on a single polynucleotide fragment so that the function of one is affected by the other. For example, a promoter is operably-linked with a coding sequence when it is capable of affecting the expression of that coding sequence (i.e., the coding sequence is under the transcriptional control of the promoter). The term “expression control sequence” (also called a regulatory sequence) refers to nucleic acid sequences that effect the expression and processing of coding sequences to which they are operably linked. For example, expression control sequences may include transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In some embodiments, a nucleic acid or an expression construct encoding an anti-PSMA antibody or antigen binding fragment thereof is present in a vector. A “vector” is a nucleic acid molecule that is capable of transporting another nucleic acid. Vectors may be, for example, plasmids, cosmids, viruses, a RNA vector or a linear or circular DNA or RNA molecule that may include chromosomal, non-chromosomal, semi-synthetic or synthetic nucleic acids. Exemplary vectors are those capable of autonomous replication (episomal vector) or expression of nucleic acids to which they are linked (expression vectors). Exemplary viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). In some embodiments, a vector is a plasmid. In some other embodiments, a vector is a viral vector. In some such embodiments, the viral vector is a lentiviral vector or a γ-retroviral vector.

In a further aspect, the present disclosure also provides an isolated host cell comprising a nucleic acid, expression construct, or vector encoding an anti-PSMA antibody or antigen binding fragment thereof as described herein. As used herein, the term “host” refers to a cell or microorganism targeted for genetic modification with a heterologous or exogenous nucleic acid molecule to produce a polypeptide of interest (e.g., an anti-PSMA antibody or antigen-binding fragment thereof). In certain embodiments, a host cell may optionally already possess or be modified to include other genetic modifications that confer desired properties related or unrelated to biosynthesis of the heterologous or exogenous protein (e.g., inclusion of a selectable marker). More than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more exogenous nucleic acid molecules are introduced into a host cell, it is understood that the two more exogenous nucleic acid molecules can be introduced as a single nucleic acid molecule (e.g., on a single vector), on separate vectors, integrated into the host chromosome at a single site or multiple sites. The number of referenced heterologous nucleic acid molecules or protein activities refers to the number of encoding nucleic acid molecules or the number of protein activities, not the number of separate nucleic acid molecules introduced into a host cell.

Examples of host cells include, but are not limited to, eukaryotic cells, e.g., yeast cells, animal cells, insect cells, plant cells; and prokaryotic cells, including E. coli. In some embodiments, the cells are mammalian cells. In some embodiments, the host cell is a human embryonic kidney (HEK293) cell, Y0 cell, Sp2/0 cell, NS0 murine myeloma cell, PER.C6® human cell, baby hamster kidney cell (BHK), COS cell, or Chinese hamster ovary (CHO) cell. Host cells are cultured using methods known in the art.

In some embodiments, the present disclosure provides a mammalian host cell comprising:
(a) a polynucleotide sequence of SEQ ID NO: 47 and a polynucleotide sequence of SEQ ID NO: 45;
(b) a polynucleotide sequence of SEQ ID NO: 51 and a polynucleotide sequence of SEQ ID NO: 49;
(c) a polynucleotide sequence of SEQ ID NO: 55 and a polynucleotide sequence of SEQ ID NO: 53;
(d) a polynucleotide sequence of SEQ ID NO: 59 and a polynucleotide sequence of SEQ ID NO: 57;
(e) a polynucleotide sequence of SEQ ID NO: 63 and a polynucleotide sequence of SEQ ID NO: 61;
(f) a polynucleotide sequence of SEQ ID NO: 67 and a polynucleotide sequence of SEQ ID NO: 65;
(g) a polynucleotide sequence of SEQ ID NO: 71 and a polynucleotide sequence of SEQ ID NO:69;
(h) a polynucleotide sequence of SEQ ID NO: 75 and a polynucleotide sequence of SEQ ID NO: 73;
(i) a polynucleotide sequence of SEQ ID NO: 79 and a polynucleotide sequence of SEQ ID NO: 77; or
(j) a polynucleotide sequence of SEQ ID NO: 83 and a polynucleotide sequence of SEQ ID NO: 81;
wherein the cell is capable of expressing an antibody or antigen-binding fragment thereof that binds human PSMA.

In some embodiment, the mammalian host cell comprises:
(a) a polynucleotide sequence encoding a polypeptide having an amino acid sequence of SEQ ID NO: 48 and a polynucleotide sequence encoding a polypeptide having an amino acid sequence of SEQ ID NO: 46;
(b) a polynucleotide sequence encoding a polypeptide having an amino acid sequence of SEQ ID NO: 52 and a polynucleotide sequence encoding a polypeptide having an amino acid sequence of SEQ ID NO: 50;
(c) a polynucleotide sequence encoding a polypeptide having an amino acid sequence of SEQ ID NO: 56 and a polynucleotide sequence encoding a polypeptide having an amino acid sequence of SEQ ID NO: 54;
(d) a polynucleotide sequence encoding a polypeptide having an amino acid sequence of SEQ ID NO: 60 and a polynucleotide sequence encoding a polypeptide having an amino acid sequence of SEQ ID NO: 58;
(e) a polynucleotide sequence encoding a polypeptide having an amino acid sequence of SEQ ID NO: 64 and a polynucleotide sequence encoding a polypeptide having an amino acid sequence of SEQ ID NO: 62;
(f) a polynucleotide sequence encoding a polypeptide having an amino acid sequence of SEQ ID NO: 68 and a polynucleotide sequence encoding a polypeptide having an amino acid sequence of SEQ ID NO: 66;
(g) a polynucleotide sequence encoding a polypeptide having an amino acid sequence of SEQ ID NO: 72 and a polynucleotide sequence encoding a polypeptide having an amino acid sequence of SEQ ID NO:70;
(h) a polynucleotide sequence encoding a polypeptide having an amino acid sequence of SEQ ID NO: 76 and a polynucleotide sequence encoding a polypeptide having an amino acid sequence of SEQ ID NO: 74;
(i) a polynucleotide sequence encoding a polypeptide having an amino acid sequence of SEQ ID NO: 80 and a polynucleotide sequence encoding a polypeptide having an amino acid sequence of SEQ ID NO: 78; or
(j) a polynucleotide sequence encoding a polypeptide having an amino acid sequence of SEQ ID NO: 84 and a polynucleotide sequence encoding a polypeptide having an amino acid sequence of SEQ ID NO: 82;
wherein the cell is capable of expressing an antibody or antigen-binding fragment thereof that binds human PSMA.

In yet another aspect, the present disclosure provides a process for making an anti-PSMA antibody or antigen binding fragment thereof as described herein, comprising culturing a host cell of the present disclosure, under suitable conditions and for a sufficient time to express the anti-PSMA antibody or antigen binding fragment thereof, and optionally isolating the anti-PSMA antibody or antigen binding fragment thereof from the culture. Purification of soluble antibodies or antigen binding fragments thereof may be performed according to methods known in the art.

### Pharmaceutical Compositions

In another aspect, the present disclosure provides a composition comprising an anti-PSMA antibody or antigen binding fragment thereof as described herein and a pharmaceutically acceptable carrier, diluent, or excipient. Pharmaceutically acceptable carriers for diagnostic and therapeutic use are well known in the pharmaceutical art, and are described, for example, in Remington's Pharmaceutical Sciences, Mack Publishing Co. (A. R. Gennaro (Ed.), 18^{th} Edition, 1990) and in CRC Handbook of Food, Drug, and Cosmetic Excipients, CRC Press LLC (S. C. Smolinski, ed., 1992). Exemplary pharmaceutically acceptable carriers include any adjuvant, carrier, excipient, glidant, diluent, preservative, dye/colorant, surfactant, wetting agent, dispersing agent, suspending agent, stabilizer, isotonic agent, solvent, emulsifier, or any combination thereof. For example, sterile saline and phosphate buffered saline at physiological pH can be suitable pharmaceutically acceptable carriers. Preservatives, stabilizers, dyes or the like may also be provided in the pharmaceutical composition. In addition, antioxidants and suspending agents may also be used. Pharmaceutical compositions may also contain diluents such as water, buffers, antioxidants such as ascorbic acid, low molecular weight polypeptides (less than about 10 residues), proteins, amino acids, carbohydrates (e.g., glucose, sucrose, dextrins), chelating agents (e.g., EDTA), glutathione, and other stabilizers and excipients. Neutral buffered saline or saline mixed with nonspecific serum albumin are exemplary diluents.

The pharmaceutical compositions described herein can be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques.

In some embodiments, pharmaceutical compositions of the present invention are formulated in a single dose unit or in a form comprising a plurality of dosage units. Methods of preparing such dosage forms are known, or will be apparent, to those skilled in this art; for example, see Remington: The Science and Practice of Pharmacy, 20th Edition (Philadelphia College of Pharmacy and Science, 2000).

A pharmaceutical composition may be in the form of a solid, semi-solid or liquid. Solid compositions may include powders and tablets. In some embodiments, the pharmaceutical compositions described here are lyophilized or in powder form for re-constitution with a suitable vehicle, e.g., sterile water, before use. In some embodiments, the pharmaceutical compositions described herein is a suspension, solution, or emulsion.

### Therapeutic Uses

The anti-PSMA antibodies or antigen-binding fragments thereof of the present disclosure may be used in a method of treating cancer, comprising administering to a patient in need thereof, an effective amount of an anti- PSMA antibody or antigen binding fragment of the present disclosure, or a pharmaceutical composition comprising an anti- PSMA antibody or antigen binding fragment of the present disclosure.

Patients or subjects that can be treated by anti-PSMA antibodies or antigen-binding fragments thereof of the present disclosure include, but are not limited to, a mammal, such as human or non-human primates (e.g., monkeys and apes), a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. In some embodiments, the subject is human. The subject can be male or female and can be any suitable age, including infant, juvenile, adolescent, adult, and geriatric.

“Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or antigen binding fragment thereof, or composition of the present disclosure, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount” of an antibody, antigen-binding fragment, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state;

delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients that result in a therapeutic effect, whether administered serially, sequentially, or simultaneously. A combination may comprise, for example, an anti-PSMA antibody or antigen binding fragment thereof and an anti-cancer agent.

An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently, which can readily be determined by a person skilled in the art.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

An anti-PSMA antibody or antigen binding fragment thereof may be administered one or more times over a given period of time. In some embodiments, a method comprises administering the anti-PSMA antibody or antigen binding fragment thereof to the subject at least 2, 3, 4, 5, 6, 7, 8, 9, 10 times, or more.

In certain embodiments, a method comprises administering the anti-PSMA antibody or antigen binding fragment thereof to the subject a plurality of times, wherein a second or successive administration is performed at about 28 days, 21 days, 14 days, 10 days, 7 days, 3 days, 1 day, or less following a first administration.

Anti-PSMA antibodies or antigen-binding fragments thereof of the present disclosure may be administered to a subject by parenteral routes. In some embodiments, anti-PSMA antibodies or antigen-binding fragments thereof are administered to a subject by subcutaneous, intravenous, intraarterial, subdural, intramuscular, intracranial, intrasternal, intratumoral, intraperitoneal, or infusion techniques.

Cancers that may be treated by the anti-PSMA antibody or antigen binding fragment thereof provided in the present disclosure include hematologic malignancies and solid tumors. In certain embodiments, the cancer is a solid tumor.

In some embodiments, the cancer is a Hodgkin's lymphoma, non-Hodgkin's lymphoma, multiple myeloma, leukemia, myelodysplastic syndrome, thymus cancer, malignant mesothelioma, pituitary tumor, thyroid tumor, melanoma, Merkel cell skin cancer, lung cancer, head and neck cancer, colorectal cancer, liver cancer, bile duct cancer, gallbladder cancer, pancreatic cancer, esophageal cancer, gastric cancer, small intestine cancer, anal cancer, kidney cancer, bladder cancer, prostate cancer, penile cancer, testicular cancer, breast cancer, ovarian cancer, cervical cancer, vaginal cancer, vulvar cancer, endometrial cancer, eye cancer, soft tissue sarcoma, hepatocellular carcinoma, brain tumor, or spinal cord tumor. In certain embodiments, the cancer is prostate cancer, lung cancer, sarcoma, breast cancer, kidney cancer, or a digestive tract malignant tumor.

In some embodiments, an anti-PSMA antibody or antigen binding fragment thereof described herein may be used in combination with one or more anti-cancer agents. In some embodiments, the one or more anti-cancer agents is administered simultaneously, separately, or sequentially.

In some embodiments, an anti-cancer agent is a cellular immunotherapy, antibody therapy, immune checkpoint inhibitor therapy, hormone therapy, chemotherapeutic, targeted cancer therapy, cytokine therapy or any combination thereof. In some embodiments, a cellular immunotherapy comprises a TCR-T cell therapy, dendritic cell therapy, or chimeric antigen receptor (CAR)-T cell therapy, or any combination thereof. In some embodiments, an antibody therapy comprises an agonistic, immune enhancing antibody. In some embodiments, an antibody therapy comprises an antibody-drug conjugate. In some embodiments, an antibody therapy comprises bevacizumab, nimotuzumab, lapatinib, cetuximab, panitumumab, matuzumab, trastuzumab, nimotuzumab, zalutumumab, alemtuzumab, rituxmiab, magrolimab, or any combination thereof. In some embodiments, an immune checkpoint inhibitor therapy targets PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, CD47, SIRPα, or any combination thereof. In some embodiments, an immune checkpoint inhibitor therapy comprises ipilimumab, tremelimumab, pidilizumab, nivolumab, pembrolizumab, durvalumab, atezolizumab, avelumab, urelumab, lirilumab, or any combination thereof. In some embodiments, a hormone therapy comprises abiraterone, anastrozole, exemestane, fulvestrant, letrozole, leuprolide, tamoxifen, or any combination thereof. In some embodiments, a cytokine therapy comprises IFNα, IL-2, IFNγ, GM-CSF, IL-7, IL-12, IL-21, IL-15, or any combination thereof.

In some embodiments, a chemotherapeutic comprises an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), a DNA repair inhibitor, or an apoptosis inducing agent.

In another aspect, the present disclosure provides method of using an anti-PSMA antibody as previously described, for cancer research and/or diagnosis of cancer. In some embodiments, the anti-PSMA antibody used for cancer research and/or diagnosis comprises a heavy chain variable region (VH) and a light chain variable region (VL), wherein:
the VH comprises an amino acid sequence that has at least 90%, 91%, 92%, 93%, 94%, 95%, 96%, 97%, 98%, or 99% identity with the amino acid sequence of SEQ ID NO: 48, and the VL comprises an amino acid sequence that has at least 90%, 91%, 92%, 93%, 94%, 95%, 96%, 97%, 98%, or 99% identity with the amino acid sequence of SEQ ID NO: 46;
the VH comprises an amino acid sequence that has at least 90%, 91%, 92%, 93%, 94%, 95%, 96%, 97%, 98%, or 99% identity with the amino acid sequence of SEQ ID NO: 52, and the VL comprises an amino acid sequence that has at least 90%, 91%, 92%, 93%, 94%, 95%, 96%, 97%, 98%, or 99% identity with the amino acid sequence of SEQ ID NO: 50;
the VH comprises an amino acid sequence that has at least 90%, 91%, 92%, 93%, 94%, 95%, 96%, 97%, 98%, or 99% identity with the amino acid sequence of SEQ ID NO: 56, and the VL comprises an amino acid sequence that has at least 90%, 91%, 92%, 93%, 94%, 95%, 96%, 97%, 98%, or 99% identity with the amino acid sequence of SEQ ID NO: 54;
the VH comprises an amino acid sequence that has at least 90%, 91%, 92%, 93%, 94%, 95%, 96%, 97%, 98%, or 99% identity with the amino acid sequence of SEQ ID NO:76, and the VL comprises an amino acid sequence that has at least 90%, 91%, 92%, 93%, 94%, 95%, 96%, 97%, 98%, or 99% identity with the amino acid sequence of SEQ ID NO: 74;
the VH comprises an amino acid sequence that has at least 90%, 91%, 92%, 93%, 94%, 95%, 96%, 97%, 98%, or 99% identity with the amino acid sequence of SEQ ID NO: 80, and the VL comprises an amino acid sequence that has at least 90%, 91%, 92%, 93%, 94%, 95%, 96%, 97%, 98%, or 99% identity with the amino acid sequence of SEQ ID NO: 78;
the VH comprises an amino acid sequence that has at least 90%, 91%, 92%, 93%, 94%, 95%, 96%, 97%, 98%, or 99% identity with the amino acid sequence of SEQ ID NO: 84, and the VL comprises an amino acid sequence that has at least 90%, 91%, 92%, 93%, 94%, 95%, 96%, 97%, 98%, or 99% identity with the amino acid sequence of SEQ ID NO: 82.

In some embodiments, the anti-PSMA antibody used for cancer research and/or diagnosis comprises a heavy chain variable region (VH) and a light chain variable region (VL), wherein the VH comprises an amino acid sequence that has at least 90%, 91%, 92%, 93%, 94%, 95%, 96%, 97%, 98%, or 99% identity with the amino acid sequence of SEQ ID NO: 72, and the VL comprises an amino acid sequence that has at least 90%, 91%, 92%, 93%, 94%, 95%, 96%, 97%, 98%, or 99% identity with the amino acid sequence of SEQ ID NO: 70.

Preferably, the anti-PSMA antibody used for cancer research and/or diagnosis comprises a VH and a VL as set forth in SEQ ID NOS: 48 and 46, respectively; in SEQ ID NOS: 52 and 50, respectively; in SEQ ID NOS: 56 and 54, respectively; in SEQ ID NOS: 76 and 74, respectively; in SEQ ID NOS: 80 and 78, respectively; in SEQ ID NOS: 84 and 82, respectively; or in SEQ ID NOS: 72 and 70, respectively.

In some embodiments, the cancer research or cancer diagnosis is for a cancer selected from: prostate cancer, lung cancer, breast cancer, kidney cancer, and a digestive tract malignant tumor. In some embodiments, the cancer research or cancer diagnosis involves a method selected from: immunohistochemical testing, serological testing, flow cytometry testing, and imaging testing.

### Recombinant PSMA Polypeptides

In another aspect, the present disclosure provides recombinant PSMA polypeptides. The recombinant PSMA polypeptides disclosed herein do not include 418-567 of full-length PSMA as set forth in SEQ ID NO: 85. The recombinant PSMA polypeptides may be useful, for example, for producing anti-PSMA antibodies.

In some embodiments, the recombinant PSMA polypeptide comprises the extracellular protease domain of PSMA of full-length PSMA. In some embodiments, the recombinant PSMA polypeptides comprises an amino acid sequence that has at least 90%, 91%, 92%, 93%, 94%, 95%, 96%, 97%, 98%, or 99% identity with the amino acid sequence of SEQ ID NO:86. In some embodiments, the recombinant PSMA polypeptide comprises the amino acid sequence of SEQ ID NO:86 or 96.

In another aspect, the present disclosure provides a fusion protein comprising a recombinant PSMA polypeptide as previously described, fused to a second polypeptide. In some embodiments, the second polypeptide comprises a protein tag for purification and/or detection of the recombinant PSMA polypeptide. In some embodiments, the protein tag is selected from a 6 X HIS tag or a small ubiquitin-like modifier (SUMO) tag. An exemplary polynucleotide encoding a SUMO tag is the polynucleotide sequence of SEQ ID NO: 97. An exemplary SUMO tag polypeptide is the polypeptide sequence of SEQ ID NO: 98.

In another aspect, the present disclosure provides a gene expression cassette comprising a gene encoding a recombinant PSMA polypeptide as previously described or a fusion protein as previously described, operably linked to an expression control sequence. In some embodiments, the expression control sequence is functional in a prokaryotic cell, such as an Escherichia coli cell.

In another aspect, the present disclosure proves an expression vector comprising a gene expression cassette as previously described. In some embodiments, the expression vector is a bacterial expression vector, such as pET-28a-SUMO.

In another aspect, the present disclosure provides a cell comprising a gene expression cassette as previously described or an expression vector as previously described. In some embodiments, the host cell comprises a prokaryotic cell, such as an E. coli cell. In some embodiments, the cell is a competent cell. “Competent cell” refers to a cell designed for high-efficiency uptake of foreign DNA. Examples of competent cell lines include the E. coli strains DH5α and TOP10.

In another aspect, the present disclosure provides a method of producing an expression vector encoding a recombinant PSMA polypeptide as previously described. An expression vector encoding a recombinant PSMA polypeptide may be produced by a method comprising:
obtaining mRNA from cells expressing PSMA;
obtaining a forward primer and a reverse primer each designed to produce an amplification product ranging from nucleotide 391 to nucleotide 1506 of human PSMA (GenBank: M99487.1);
conducting a reverse transcription polymerase chain reaction in the presence of the mRNA, the forward primer, and the reverse primer, to obtain cDNA of a PSMA fragment;
subjecting the cDNA of the PSMA fragment and a prokaryotic expression vector to restriction endonuclease reactions, respectively; and
ligating the cDNA of the PSMA fragment to the prokaryotic expression vector.

An exemplary method is described in Example 1.

In some embodiments, the method further includes screening for successful ligation to obtain a recombinant expression vector containing the PSMA fragment.

In another aspect, the present disclosure provides a method of producing a cell expressing the recombinant PSMA polypeptide as previously described or a fusion protein as previously described. The method may include transfecting the cell with an expression vector as previously described.

In another aspect, the present disclosure provides a method of producing a recombinant PSMA polypeptide. The method may include culturing a cell comprising a polynucleotide encoding a recombinant PSMA polypeptide under conditions suitable for the expression of the recombinant PSMA polypeptide.

In another aspect, the present disclosure provides compositions comprising a recombinant polypeptide as previously described or a fusion protein as previously described. Such compositions may be useful, for example, for administering to a mammal to induce production of antibodies that bind to the recombinant PSMA polypeptide. In some embodiments, the compositions further include at least one excipient. In some embodiments, the at least one excipient is selected from culture medium, saline, and phosphate buffer. In some embodiments the composition is in the form of a liquid, such as an aqueous solution, or a powder, such as a lyophilized powder.

In some aspects, the present disclosure provides a method of producing antibodies comprising administering to a mammal a recombinant PSMA polypeptide as previously described, a fusion protein as previously described, or a composition comprising the same. In some embodiments, the method further comprises collecting the antibodies. The antibodies may be collected following the administering, after a time period sufficient for the mammal to produce the antibodies.

### EXAMPLES

### Example 1

### Cloning of a Recombinant PSMA Polypeptide

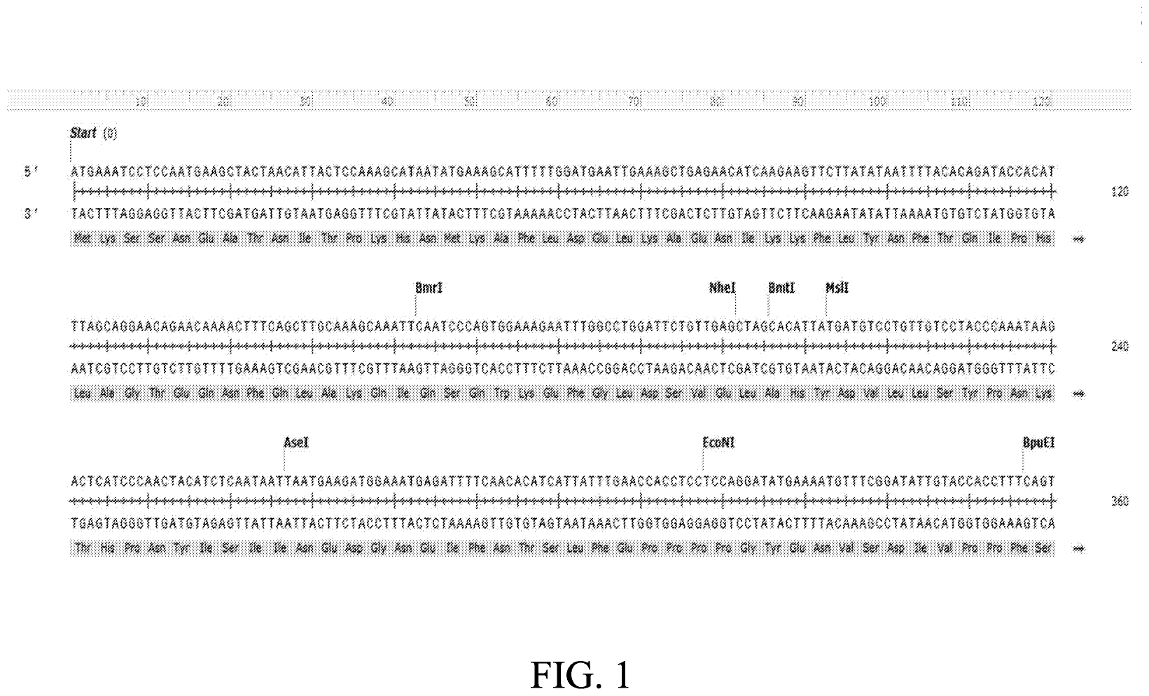

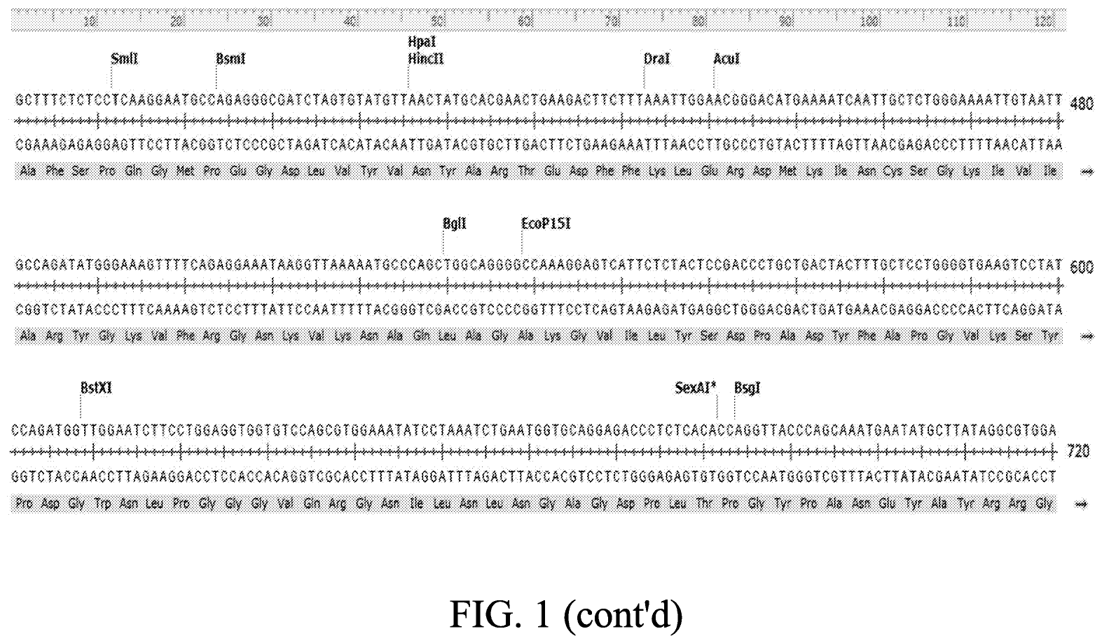

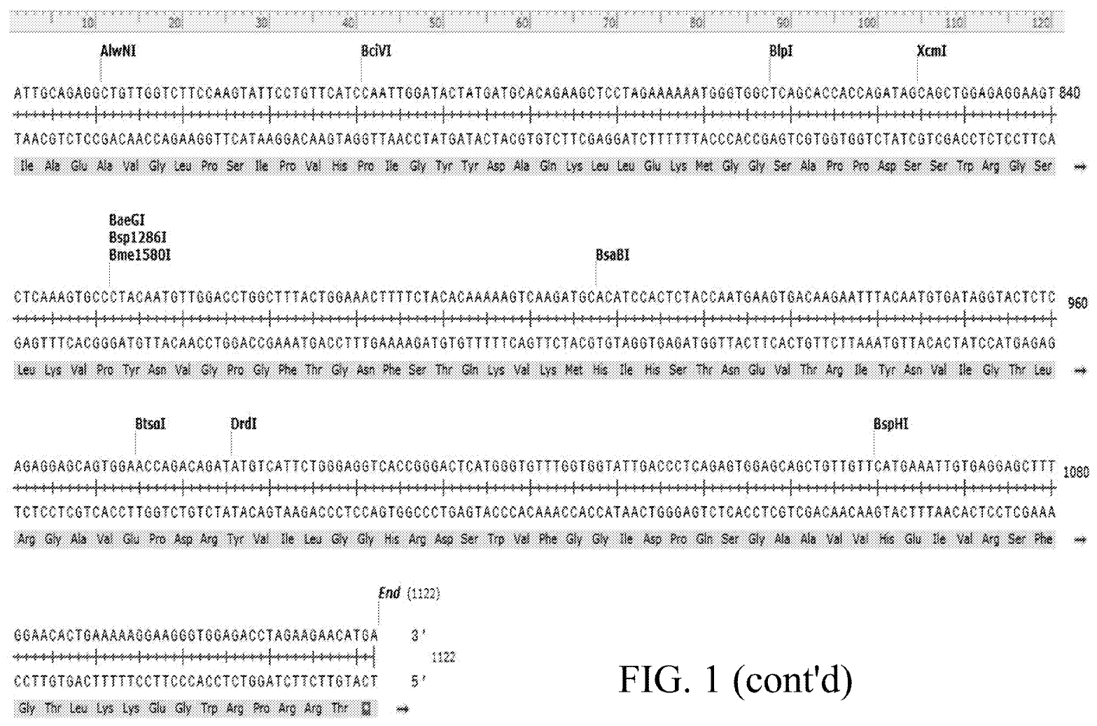

This Example demonstrates cloning of a gene encoding a recombinant PSMA polypeptide into an expression vector, and expression of the recombinant PSMA polypeptide. The recombinant PSMA polypeptide cloned and expressed in this example (shown in FIG. 1) is truncated in comparison to full-length PSMA, and includes the 44^{th} amino acid (at the N-terminus) to the 372^{nd} amino acid (at the C-terminus) of PSMA. A methionine is added at its N-terminus. To amplify the truncated form of PSMA from human mRNA, primers for RT-PCR were designed as follows:
positive strand primer: 5′-ccggaattcatg aaatcctccaatgaagctac3′ (SEQ ID NO: 87)
negative strand primer: 5′—cccaagcttctca tgttcttctaggtctccacc-3′ (SEQ ID NO: 88). A linker sequence (5′-ccggaattcatg-3′, SEQ ID NO: 90) was added to the 5′-end of the positive strand primer, including protective bases (ccg), a recognition sequence (gaattc) for EcoR I restriction endonuclease, and a start codon (atg).

A linker sequence (5′-cccaagcttctca-3′, SEQ ID NO: 91) was added to the 5′-end of the negative strand primer, including protective bases (ccc), a recognition sequence (aagctt) for Hind III restriction endonuclease, a protective base (c), and a stop codon (tca).

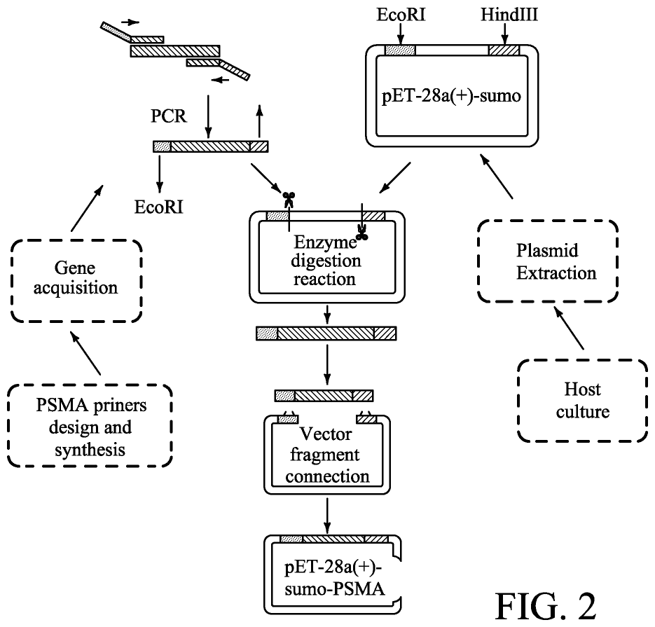

As shown in FIG. 2, the construction of the prokaryotic recombinant protein expression vector (pET-28a-SUMO/PSMA) containing the protease domain gene of PSMA was divided into the following steps:

1. Acquisition of PSMA cDNA

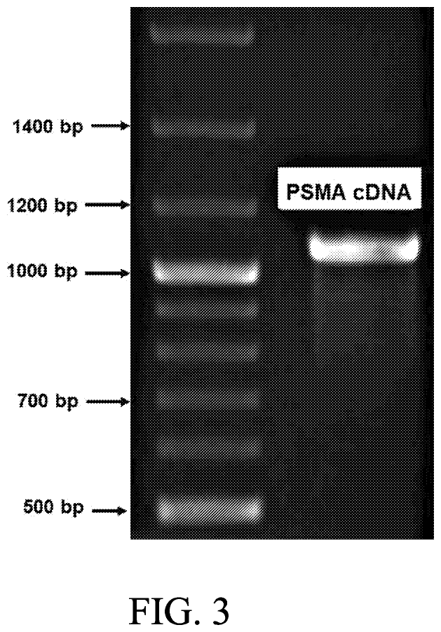

The total mRNA was obtained from a human prostate cancer tissue according to a conventional method, and a reverse transcription reaction was carried out using Oligo dT₁₅ as a primer to obtain the total cDNA. The total cDNA concentration was adjusted to 100 ng/μL. A high fidelity PCR (New England BioLabs, USA) was carried out using 2 μL of cDNA as a template under the guidance of the above PSMA primers, to amplify cDNA for the protease domain of PSMA. The PCR amplification conditions were: first 94° C. for 4 minutes; then 94° C. for 30 seconds, 58° C. for 30 seconds, and 72° C. for 90 seconds, for a total of 30 cycles; and finally 72° C. for 8 minutes. After the reaction was over, the PCR products were subjected to 1.2% sepharose gel electrophoresis detection. The detection results were as shown in FIG. 3, in which a specific band with a length of 1141 bp appeared (in which the PSMA cDNA was 1116 bp). It showed that the PSMA cDNA was successfully obtained.

2. Restriction Endonuclease Reaction

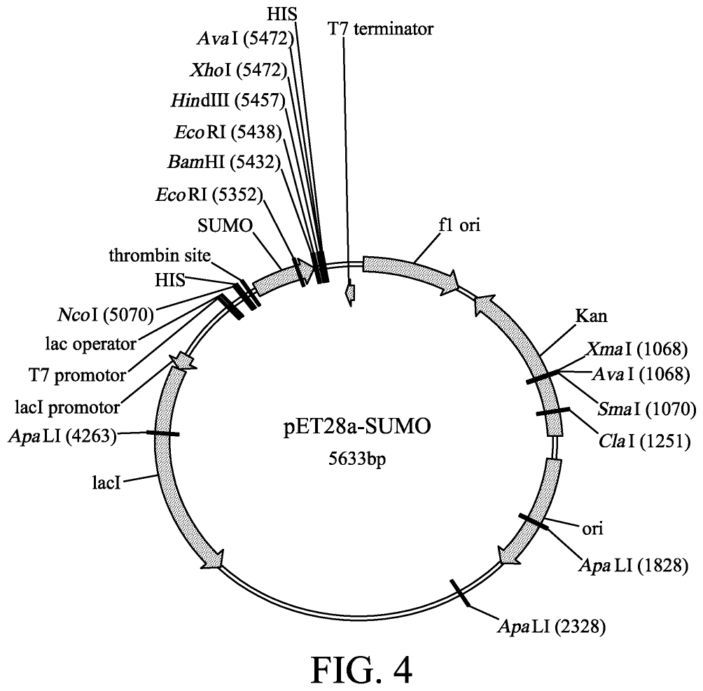

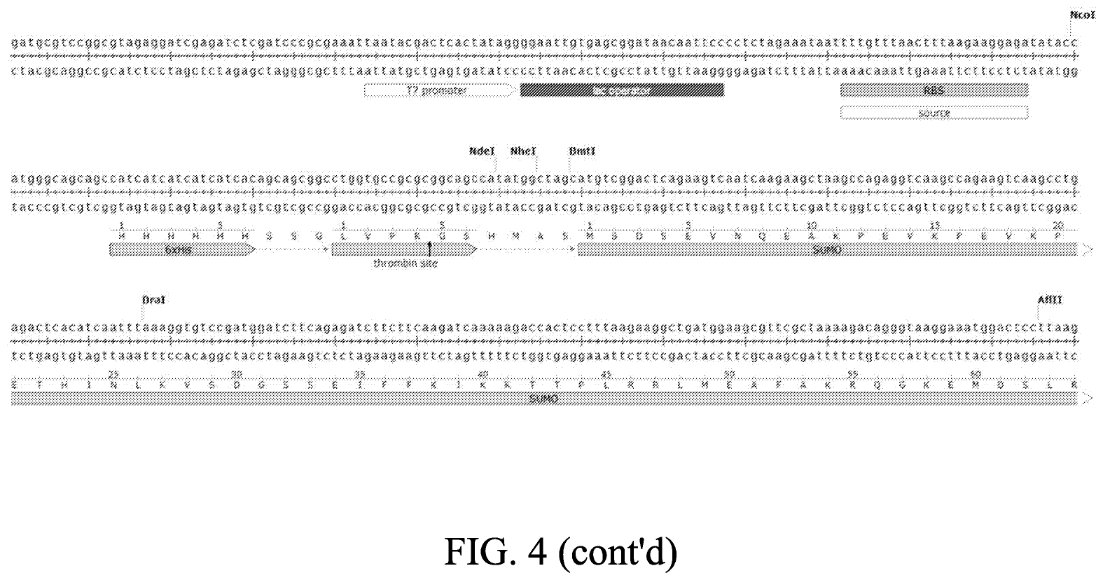

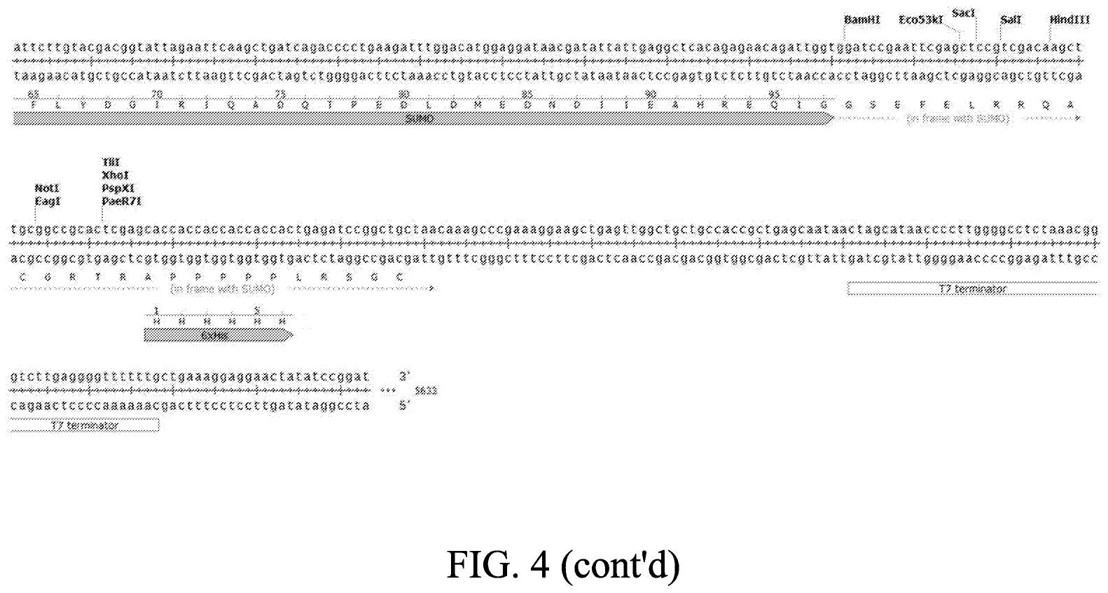

Restriction endonuclease reactions were carried out on the 1141-bp PSMA cDNA and a prokaryotic protein expression plasmid pET-28a-SUMO (FIG. 4) with restriction endonucleases EcoR-I and Hind III (Promega, USA), respectively. The digestion reaction system was: 100 ng of the pET-28a-SUMO plasmid or 50 ng of the PSMA cDNA, 10 U of EcoR-I, 10 U of Hind III, 2.5 μL of a 10× buffer, and deionized water, in a reaction volume of 25 μL. The enzyme digestion reaction conditions were: water bath at 37° C. for 4 hours.

3. DNA Ligation Reaction

After the restriction endonuclease reaction was over, a DNA ligation reaction was carried out, that is, the PSMA cDNA was inserted into the pET-28a-SUMO plasmid. The ligation reaction system was: 20 ng of the digested pET-28a-SUMO plasmid, 10 ng of the PSMA cDNA, 5 U of T4 DNA ligase (Promega, USA), 1.5 μL of a 10× T4 DNA ligation buffer, and 11.5 μL of deionized water. The reaction volume was 15 μL. The ligation reaction conditions were: 16° C. for 12 hours.

4. Screening of Recombinant pET-28a-SUMO Plasmid

The resultant recombinant plasmid was transfected into genetically engineered E. coli DH5α competent cells, LB plates containing 100 μg/mL kanamycin were used for resistance screening, single white colonies were picked, and the plasmid was extracted and purified, to obtain a prokaryotic recombinant protein expression vector (plasmid) (pET-28a-SUMO/PSMA) carrying the protease domain gene of human PSMA.

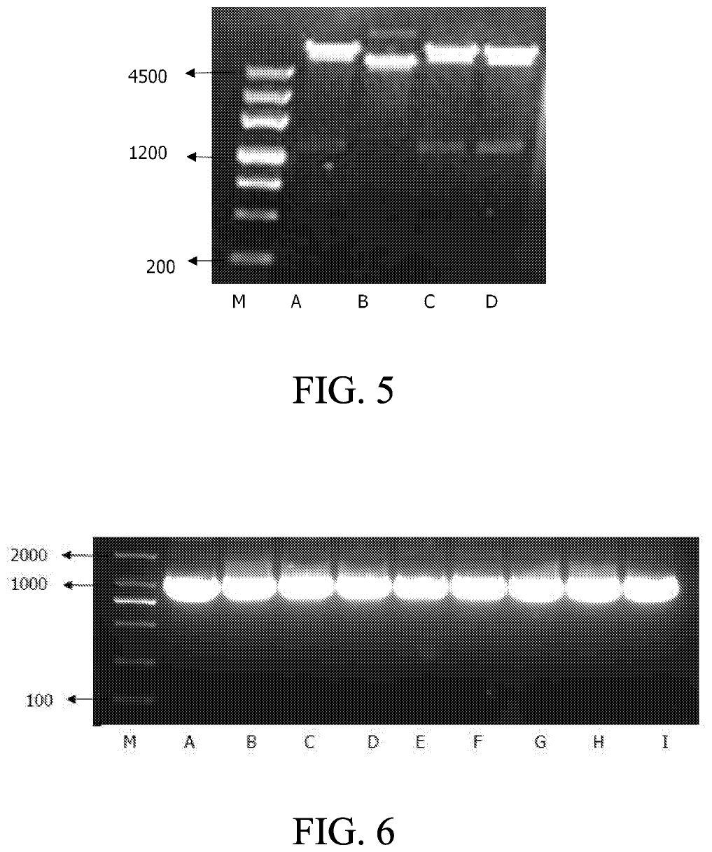

FIG. 5 shows identification results of the restriction endonuclease reaction of the pET-28a-SUMO/PSMA plasmid.

The pET-28a-SUMO/PSMA plasmid obtained by DH5α amplification was subjected to a restriction endonuclease reaction to verify whether the size of the inserted PSMA cDNA was correct. The method and conditions of the reaction were as described above. After the reaction was over, gel electrophoresis was performed. The results indicated that the size and position of the inserted PSMA cDNA were correct.

FIG. 6 shows PCR identification results of the PSMA cDNA in the pET-28a-SUMO/PSMA plasmid.

The pET-28a-SUMO/PSMA plasmid obtained by DH5α amplification was used as a template and the aforementioned PSMA cDNA primers were used to perform PCR identification. The PCR products were subjected to gel electrophoresis, and a 1141-bp specific band appeared. It proved that the construction of the pET-28a-SUMO/PSMA plasmid was successful.

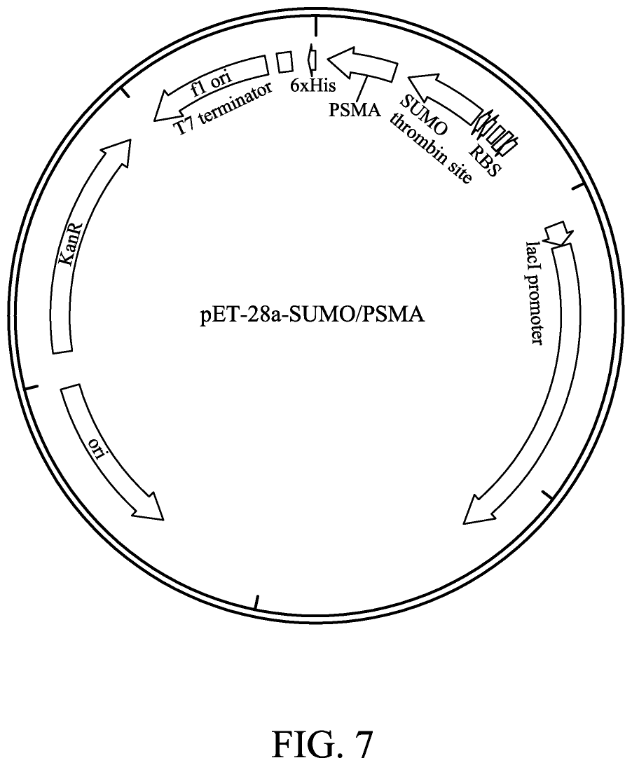

A plasmid map showing the prokaryotic recombinant protein expression vector (plasmid) (pET-28a-SUMO/PSMA) carrying the protease domain gene of human PSMA is shown in FIG. 7.

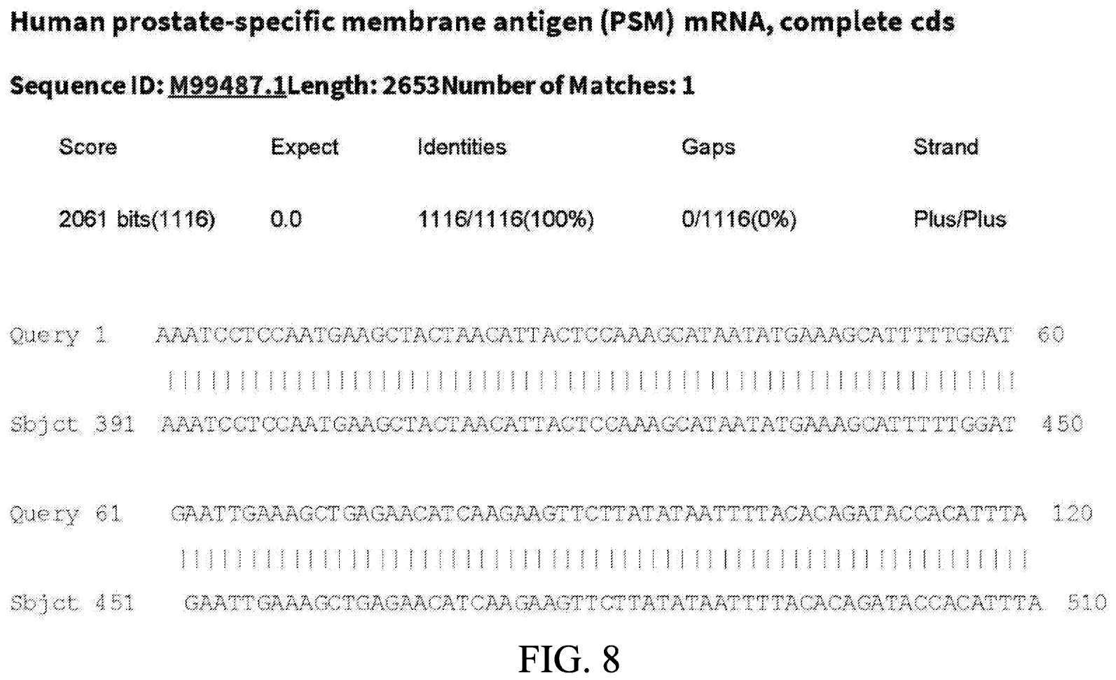

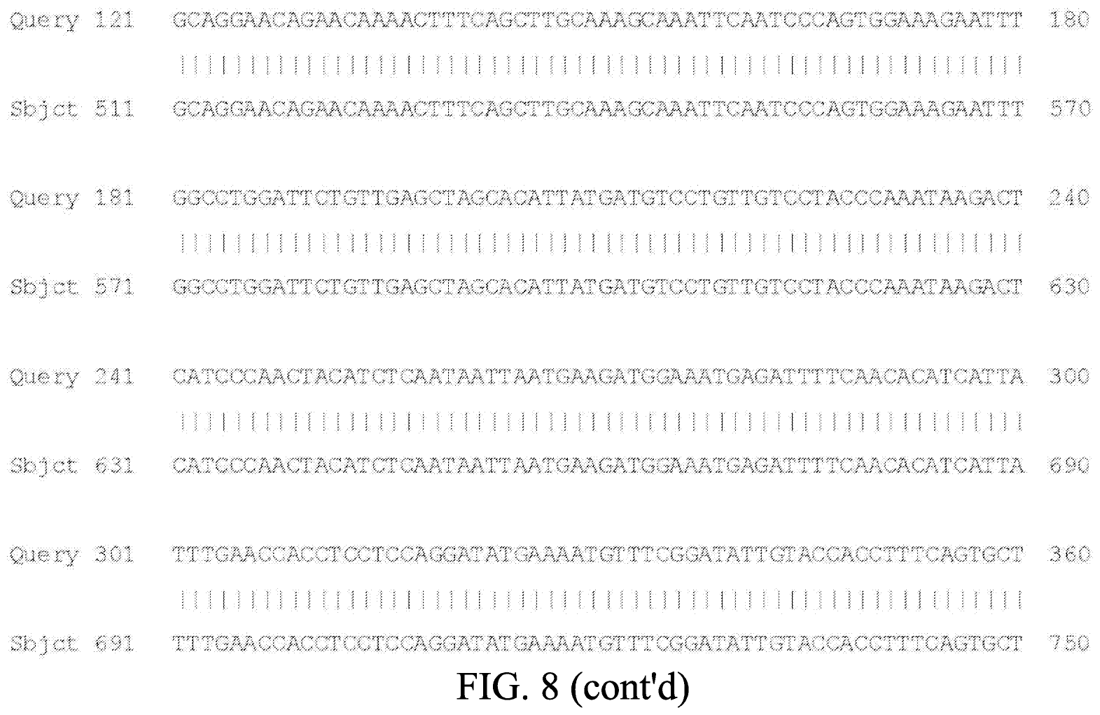

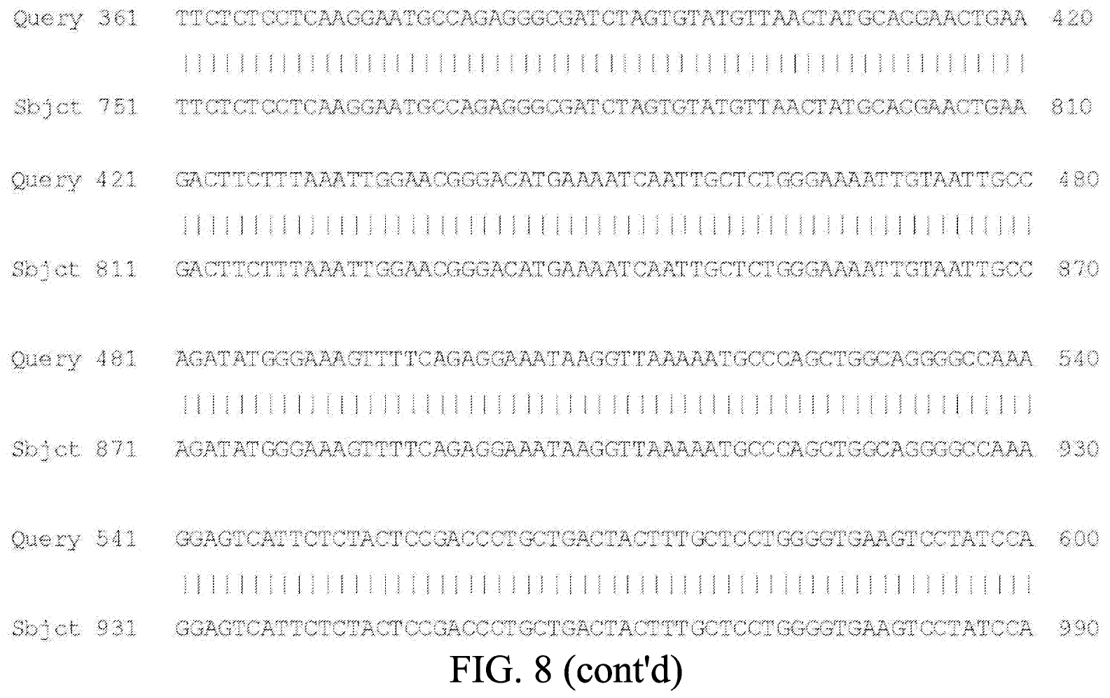

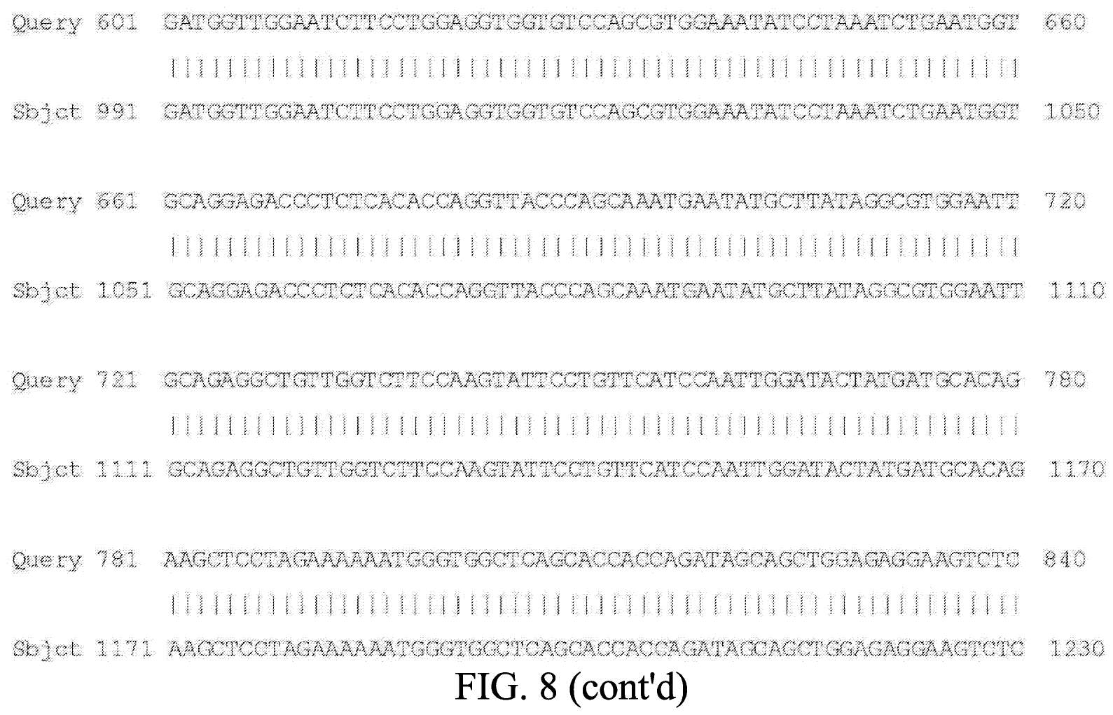

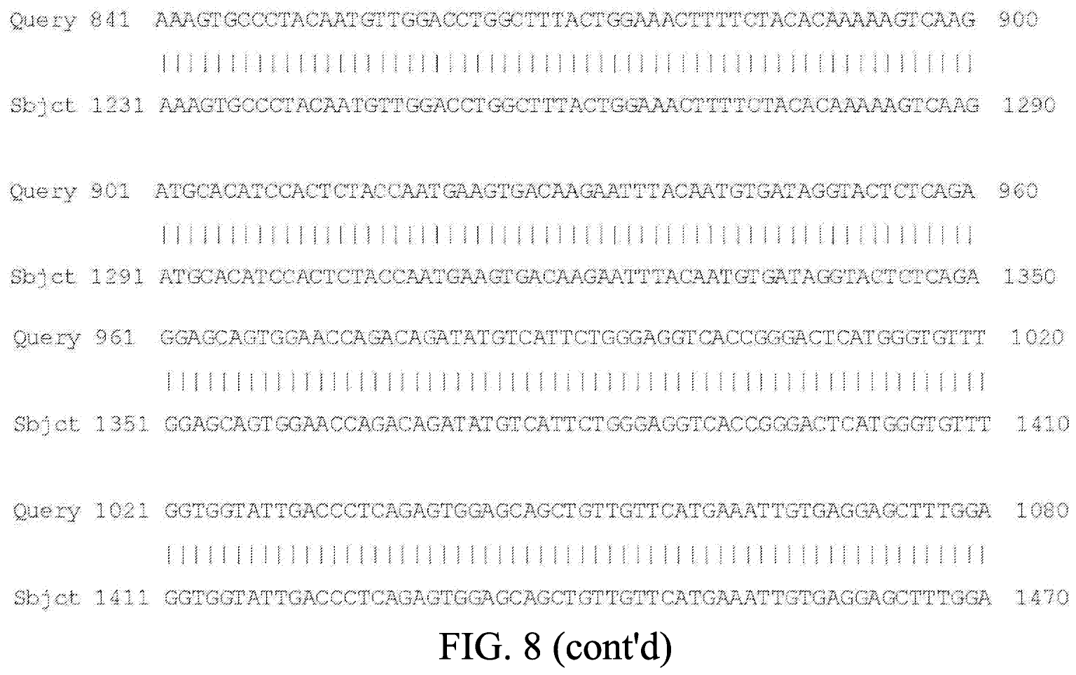

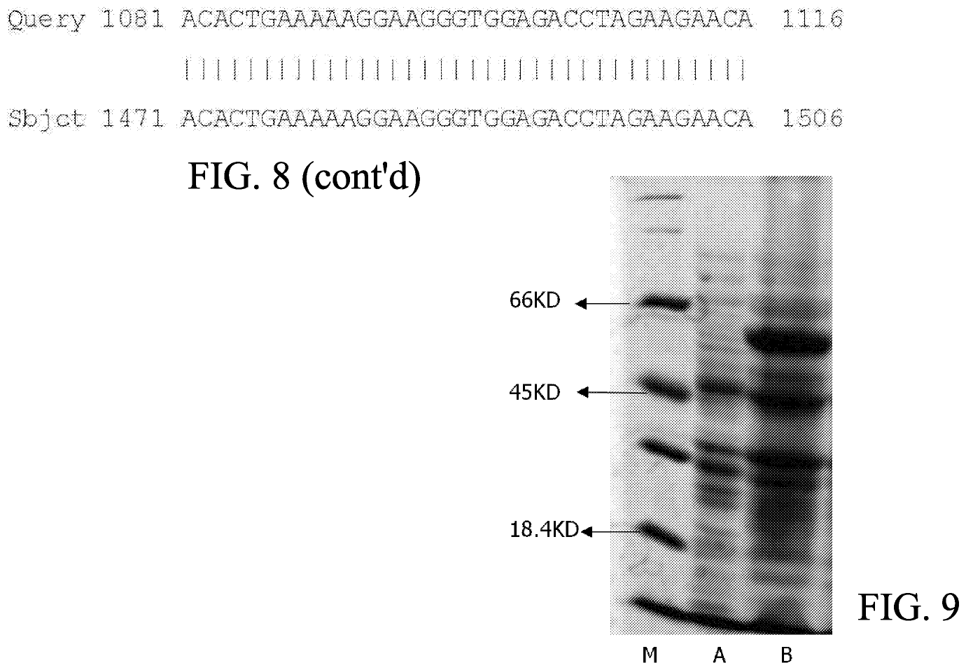

FIG. 8 shows DNA sequencing results of the PSMA cDNA in the pET-28a-SUMO/PSMA vector.

In order to verify whether the gene sequence of the PSMA cDNA inserted into the prokaryotic protein expression vector pET-28a-SUMO is correct, Invitrogen (USA) was commissioned to perform DNA sequencing. The sequencing results showed that its gene sequence was consistent with the human PSMA mRNA gene sequence provided by GenBank: M99487.1. It proved that the construction of the prokaryotic recombinant protein expression vector (pET-28a-SUMO/PSMA) carrying the protease domain gene of PSMA was completely correct.

### Example 2

### Expression and Purification of a Recombinant PSMA Polypeptide

This example demonstrates the expression and purification of the recombinant PSMA polypeptide as described in Example 1.

A cryopreservation tube containing 0.5 mL of competent cells BL21 (DE3) stored at −80° C. was thawed on ice for 5 minutes. 100 ng of the pET-28a-SUMO/PSMA vector (plasmid) was quickly added into the competent cells. The mixture was gently mixed and was put on ice for standing for 30 minutes. The mixture was placed in a 42° C. water bath, taken out after 90 seconds, and immediately placed in an ice bath for 3 minutes. Subsequently, 1 mL of an LB medium was added to the tube, and the tube was resuscitated in a shaker at 37° C. and 220 rpm for 1 h. The bacterial solution was centrifuged, part of the supernatant was discarded, and 200 μL of the bacterial solution was left, which was evenly smeared on a solid LB petri dish containing kanamycin after even mixing, placed in a constant temperature incubator at 37° C., and inverted for culture overnight.

After monoclonal colonies grew on the plate, two round colonies of moderate sizes were picked out and added into a prepared 5-mL LB medium containing kanamycin, and cultivated at 37° C. and 220 rpm until the OD value was about 0.6. At this time, an inducer IPTG (Merck, USA) was added to the culture medium at a ratio of 1/1000, to conduct induction in a constant temperature shaker at 25° C. for 16 hours. The bacteria liquid was collected and centrifuged at 8,000 rpm for 10 min at 4° C., the supernatant was discarded, and the bacteria were collected into a 1.5 mL centrifuge tube. A certain amount of deionized water was added to the centrifuge tube, and after through mixing, the mixture was placed in an ice-water mixture for ultrasonic lysis. The bacteria was lysed in a cell sonicator at 30% intensity for 30 minutes at a frequency of lysing for 3 seconds and stopping for 5 seconds. Centrifugation was performed at 4° C. at 8,000 rpm for 8 min, the supernatant and the precipitate were collected respectively, and the precipitate was resuspended with a small amount of deionized water. 100 μL of an uninduced bacterial solution, the total protein after induction, and the supernatant and precipitate after sonication were respectively put into 1.5-mL centrifuge tubes, 25 μL of 5× SDS-PAGE loading buffers were added, and the centrifuge tubes were placed in boiling water for 5-10 min. Through protein electrophoresis detection, it was found that compared with the uninduced sample, the protein was significantly expressed at about 66 kD after induction, and the molecular weight was consistent with an expected result. FIG. 9 shows the gel electrophoresis detection of PSMA protein expression after induction of thepET-28a-SUMO/PSMA vector.

In order to analyze the solubility of the recombinant PSMA protein expressed by pET-28a-SUMO/PSMA, after sonication, the proteins were separated into a supernatant and a precipitate, and electrophoresis analysis was performed thereon. It was found that the recombinant protein was mainly present in the form of inclusion bodies.

Since the recombinant PAMA protein was expressed in the form of inclusion bodies, further purification was required.
A. When the cells were lysed by sonication, deionized water was added to perform sonication lysis, and then centrifugation was performed at 4° C. and 8000 rpm for 8 min. After the supernatant was discarded, an inclusion body dissolving solution was added to resuspend and dissolve the inclusion bodies. The centrifuge tube was placed on ice and shaken in a shaker for 5 hours to fully dissolve the inclusion bodies. The impurities in the precipitate were filtered out with a 0.45-μM filter membrane.
B. A nickel (Ni) column carrying a 6xHis tag recombinant protein for purification (GE, USA) was prepared according to the instructions provided by the manufacturer, 3 mL of Ni was injected into the 15-mL column, the supernatant was discarded, then the column was washed with deionized water 5 times, and the Ni column was balanced with a binding buffer containing 8 M urea.
C. The lysed inclusion body solution was added to the Ni column, and the column was placed on ice for binding in an overturning shaker for 8 hours. After the solution in the Ni column was collected, 1 mL of the binding buffer was added to wash the column.
D. 1 mL of an elution buffer was added to the Ni filler, and was placed on ice and shaken in a shaker for 5 minutes, and the eluate was collected and stored at 4° C.

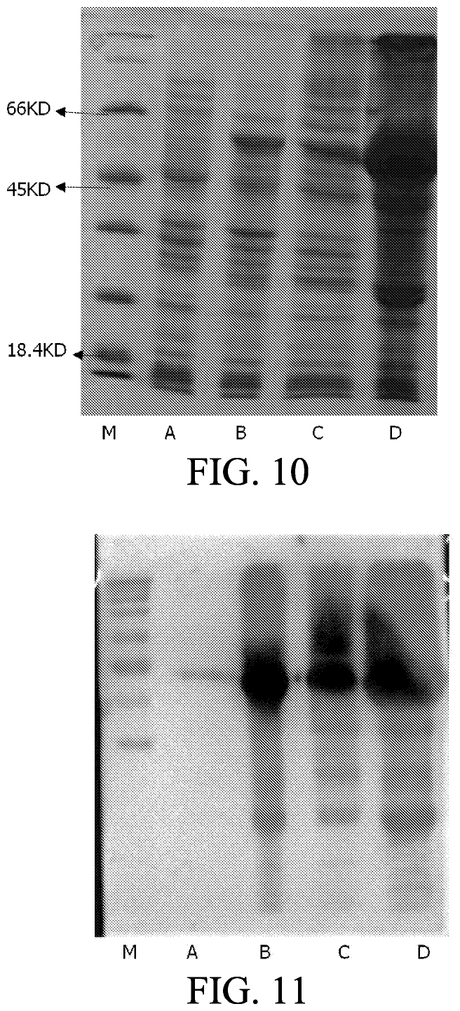

The eluate was analyzed by SDS-PAGE and the results are shown in FIG. 10, which indicates that recombinant PSMA protein was successfully purified.

In order to confirm that the recombinant protein expressed by the pET-28a-SUMO/PSMA vector after induction was a His-PSMA fusion protein, Western blotting was employed for identification using a 6xHis monoclonal antibody (Mouse anti-6*His tag monoclonal antibody). The results of the Western blot are shown in FIG. 11. The results showed that the expressed recombinant protein was a His-PSMA fusion protein, and the protein expression was successful. The experimental steps for the Western blot analysis were as follows:
A. After the expressed PSMA recombinant protein was centrifuged, the supernatant was discarded, and the precipitate was dissolved with deionized water, 100 μL of which was then taken and added into a 1.5-mL centrifuge tube, and 25 μL of a 5× loading buffer was added thereto. The protein sample was thoroughly mixed and then boiled for 10 minutes. An uninduced sample was used as a control on which the same steps were performed.
B. SDS-PAGE electrophoresis gel: the separation gel concentration was 12%, and the concentration gel concentration was 5%. The protein sample prepared above was added to prepared protein gel wells, with a sample volume of 4 μL per well, and 5 μL of a protein marker was added at the same time as a standard control.
C. After the protein electrophoresis was completed, the electrophoresis gel was placed in a transfer buffer for subsequent use. To activate a PVDF membrane (BioRad, USA), the PVDF membrane was soaked in a methanol solution for 1 minute. The protein was transferred to the membrane at 4° C. and 260 mA for 60 minutes according to the instructions of a protein-to-membrane transfer device. After the protein was transferred from the electrophoresis gel to the PVDF membrane, the membrane was soaked in a 5% blocking solution (skimmed milk powder) for 3 hours at room temperature.
D. A 6xHis monoclonal antibody (Mouse anti-6*His tag monoclonal antibody) (Invitrogen, USA) was added into the blocking solution according to the operating instructions provided by the manufacturer, and the PVDF membrane was placed therein at 4° C. overnight. The membrane was placed at room temperature. After 1 hour, the membrane was washed with a TBST solution 6 times for 5 minutes each time.
E. An HRP-labeled goat anti-mouse (IgG) secondary antibody (Abcam, USA) was added to the blocking solution according to the operating instructions provided by the manufacturer, and the PVDF membrane was placed therein at room temperature for 3 hours, and then the membrane was washed with a TBST solution 6 times, 5 minutes each time.
F. The washed membrane was developed using a chemical development method, and was observed and photographed in a gel imaging system (FIG. 11).

The protein was expressed in inclusion bodies which requires the use of strong solvents to solubilize the protein, causing the protein to be in an unfolded, denatured state with no biological activity. Therefore, in order to obtain a biologically active protein, following solubilization of the inclusion bodies were solubilized, the protein therein was renatured. In this experiment, the recombinant PSMA protein expressed by pET-28a-SUMO/PSMA was renatured by gradient dialysis. After the recombinant PSMA protein was dissolved with 8 M urea, it was refolded by dialysis. The recombinant PSMA protein was sequentially dialysed with 6 M urea, 0.1 M Tris-HCl, 4 M urea, 0.1 M Tris-HC1, 2 M urea, and 0.1 M Tris-HCl for 4 hours, and then 0.0 M urea and 0.1 M Tris-HCl, and stored at room temperature overnight.

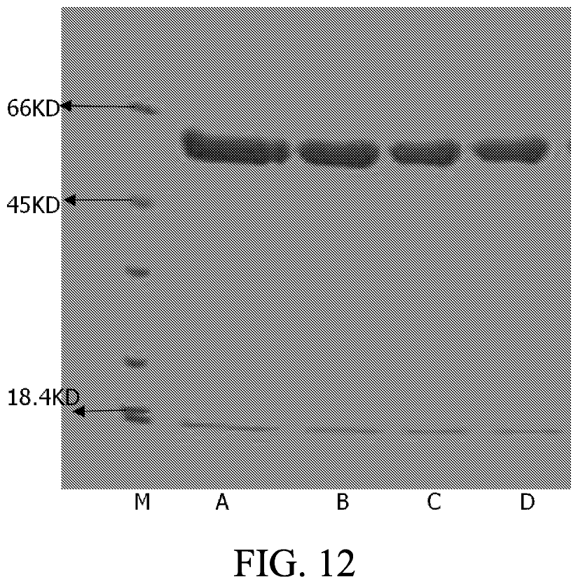

FIG. 12 shows identification results of a renatured purified protein from the inclusion bodies. As shown in FIG. 12, the purified and renatured recombinant protein expressed by pET-28a-SUMO/PSMA had a higher concentration, which is useful for subsequent research and application.

### Example 4

### Production of Anti-PSMA Antibodies

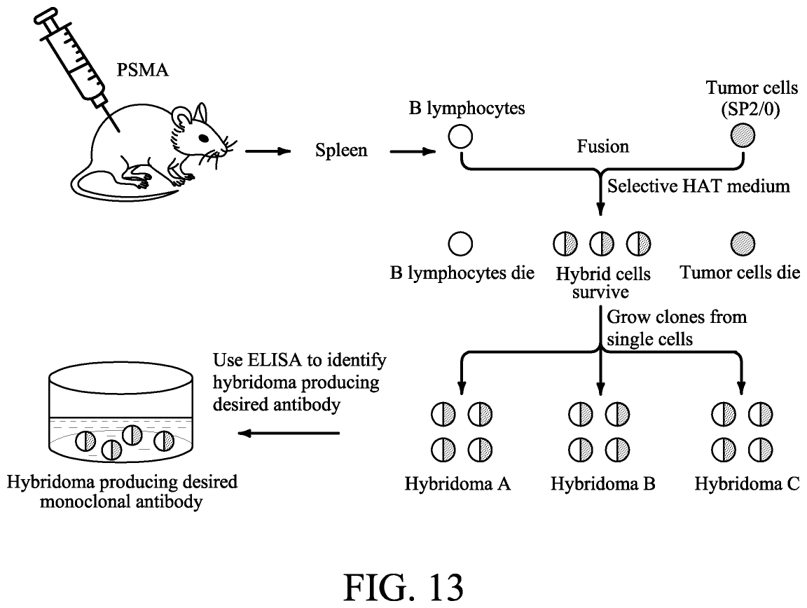

The purified, recombinant PSMA polypeptide as described in Example 3 was used as an antigen for the production of anti-PSMA antibodies. To obtain the monoclonal antibodies (PSMA-McAb) against the protease domain of PSMA, as shown in FIG. 13, the following steps were used:

A. Animal Immunity

The purified protein containing the protease domain of PSMA was used as an antigen and mixed well with a Freund's complete adjuvant (Sigma-Aldrich, USA) and injected into four healthy BALB/c mice of 8 weeks old subcutaneously at multiple sites. The first dose was 100 μg. The second dose was 100 μg, in which the antigen was mixed well with a Freund incomplete adjuvant (Sigma-Aldrich, USA) and injected subcutaneously at multiple sites. The third dose was 180 μg, in which the antigen was mixed well with sterile water and injected into the abdominal cavity. The immunization interval was 28 days. 14 days after the third immunization, blood was collected from the tail vein to test the antibody titer. The antibody titer in the serum was 10⁻⁷. 3 days before cell fusion, the antigen was well mixed with sterile water and injected in an amount of 50 μg into the abdominal cavity to strengthen immunity.

B. Cell Fusion

The spleens of the mice were harvested by aseptic operation, to prepare spleen cells. The spleen cells was mixed with myeloma cells (SP2/0) at a ratio of 1:5. After centrifugation, the supernatant was discarded, and 50% PEG 4000 in volume fraction was slowly added for fusion. The fused cells were resuspended with a 1% volume fraction (HAT:DMEM) HAT culture fluid (Sigma-Aldrich, USA) and added into a 96-well plate, 10⁵ cells per well/100 μL, which was placed in a CO₂ cell incubator for culture, during which cell growth statuses were recorded every day, and the 1% HAT (HAT:DMEM) culture fluid was replenished.

C. Screening and Cloning of Hybridoma Cells

When there were fused cells growing in the 96-well plate and the fluid turned yellow slightly, the culture fluid was drawn and screened using an indirect ELISA method for coated recombinant proteins, to select strong positive wells in which a limiting dilution method was used for subcloning. Monoclonal antibodies were obtained after 3 times of subcloning.

D. Acquisition of Hybridoma Cells

After the spleen cells of the immunized mice were fused with Sp2/0 and screened, 18 strains of cells that stably secreted antibodies and had high titers were obtained, which were propagated and cryopreserved.

E. Preparation of Ascites Containing Monoclonal Antibodies

10 weeks old Health BALB/c mice were intraperitoneally injected with sterilized liquid paraffin 0.5 mL/animal followed by intraperitoneal injection of hybridoma 0.5 mL (5×10⁵-1×10⁶/hybridoma cells/animal) 7-14 days later. When the abdominal cavity of the mice obviously enlarged, the ascites was drawn, and was cryopreserved at −80° C. after removal of grease and sediment for later use.

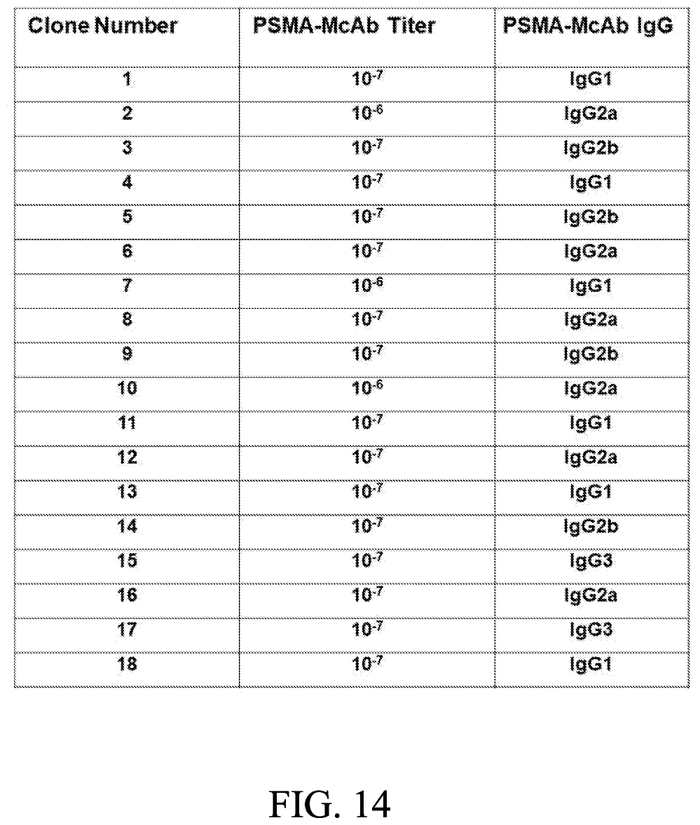

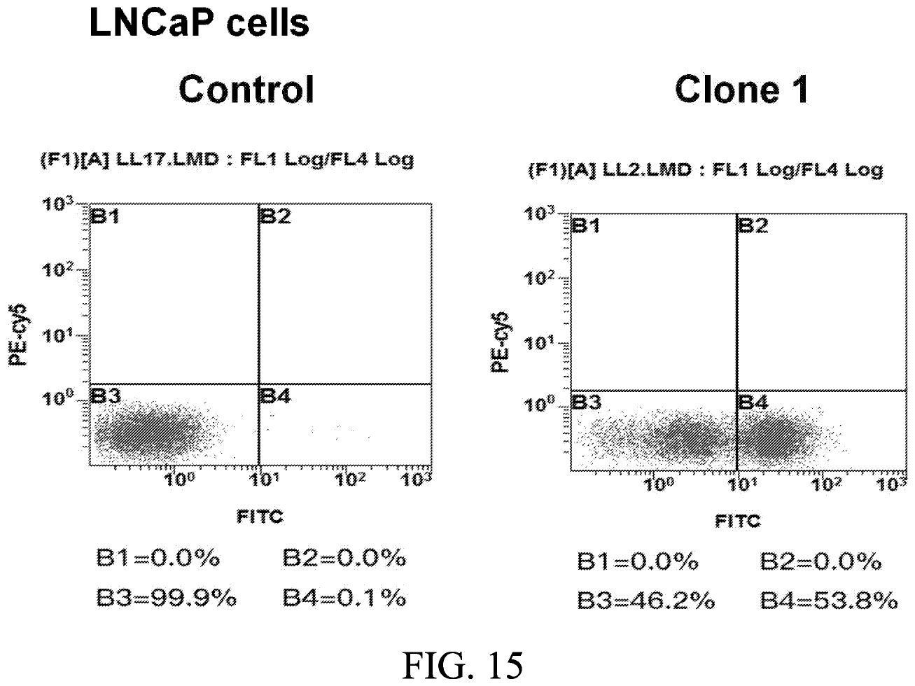

FIG. 14 shows the titers and subtypes of the 18 clones of the PSMA-McAb obtained.

Indirect ELISA was employed to detect the titer of monoclonal antibodies in the mouse ascites. A Sigma monoclonal antibody typing kit was used to identify IgG monoclonal antibodies in the ascites. The results were shown in FIG. 14. The titers of the 18 clones of the PSMA-McAb obtained ranged from 1×10⁻⁶-1×10⁻⁷. The subtypes of the IgG antibodies were IgG1, IgG2a, IgG2b, and IgG3, respectively.

The CDR sequences and VH and VL sequences of some of the antibodies obtained are described below:

| No. 1 | CDR1 | CDR2 | CDR3 |
|---|---|---|---|
| Light Chain | KSVSTSGYSY | LVS | QHIRELTR |
| | (SEQ ID NO: 1) | (SEQ ID NO: 2) | (SEQ ID NO: 3) |
| Heavy Chain | GYTFTDYY | INPYNGGT | ARSYRYDRAWFAY |
| | (SEQ ID NO: 4) | (SEQ ID NO: 5) | (SEQ ID NO: 6) |
| No. 6 | CDR1 | CDR2 | CDR3 |
| Light Chain | QSLLDSDGKTY | LVS | WQGTHFPRT |
| | (SEQ ID NO: 7) | (SEQ ID NO: 2) | (SEQ ID NO: 8) |
| Heavy Chain | YTFSRRVYFAIRDTN | IYPGNGDT | ACDYYYAMDY |
| | YW | (SEQ ID NO: 10) | (SEQ ID NO: 11) |
| | (SEQ ID NO: 9) | | |
| No. 7 | CDR1 | CDR2 | CDR3 |
| Light Chain | KSVSTSGYSY | LVS | QHIRELTR |
| | (SEQ ID NO: 1) | (SEQ ID NO: 2) | (SEQ ID NO: 3) |
| Heavy Chain | GFTFSDYR | IKRKSDNYGT | GRGLAEGFPY |
| | (SEQ ID NO: 12) | (SEQ ID NO: 13) | (SEQ ID NO: 14) |
| No. 8 | CDR1 | CDR2 | CDR3 |
| Light Chain | KSVSTSGYSY | LVS | QHIRELTR |
| | (SEQ ID NO: 1) | (SEQ ID NO: 2) | (SEQ ID NO: 3) |
| Heavy Chain | GFTFSNYA | ISSGGSYT | AREGWEGY |
| | (SEQ ID NO: 15) | (SEQ ID NO: 16) | (SEQ ID NO: 17) |
| No. 9 | CDR1 | CDR2 | CDR3 |
| Light Chain | KSVSTSGYSY | LVS | QHIRELTR |
| | (SEQ ID NO: 1) | (SEQ ID NO: 2) | (SEQ ID NO: 3) |
| Heavy Chain | GFTFSDYY | ISDGGSYT | ARRGLLNSLLRPPYAMDY |
| | (SEQ ID NO: 18) | (SEQ ID NO: 19) | (SEQ ID NO: 20) |
| No. 11 | CDR1 | CDR2 | CDR3 |
| Light Chain | QSISNN | YAS | QQSNSWPLT |
| | (SEQ ID NO: 21) | (SEQ ID NO: 22) | (SEQ ID NO: 23) |
| Heavy Chain | VYTFTSYW | IFPGTGTT | ARYGGNQYYYAMDY |
| | (SEQ ID NO: 24) | (SEQ ID NO: 25) | (SEQ ID NO: 26) |
| No. 12 | CDR1 | CDR2 | CDR3 |
| Light Chain | QSLLNSGNQKSY | WAS | QNDYSYPLT |
| | (SEQ ID NO: 27) | (SEQ ID NO: 28) | (SEQ ID NO: 29) |
| Heavy Chain | GYSITSDYA | ISYSGST | ARQTYYYGSSPFTY |
| | (SEQ ID NO: 30) | (SEQ ID NO: 31) | (SEQ ID NO: 32) |
| No. 14 | CDR1 | CDR2 | CDR3 |
| Light Chain | QSVSND | YAS | QQDYSSPYT |
| | (SEQ ID NO: 33) | (SEQ ID NO: 22) | (SEQ ID NO: 34) |
| Heavy Chain | GFSLTSYG | IWRGGSTD | AKKKYGNYGAMDY |
| | (SEQ ID NO: 35) | (SEQ ID NO: 36) | (SEQ ID NO: 37) |
| No. 15 | CDR1 | CDR2 | CDR3 |
| Light Chain | KSVSTSGYSY | LVS | QHIRELTR |
| | (SEQ ID NO: 1) | (SEQ ID NO: 2) | (SEQ ID NO: 3) |
| Heavy Chain | GYTFSSYW | ILPGSGST | ATT |
| | (SEQ ID NO: 38) | (SEQ ID NO: 39) | (SEQ ID NO: 40) |
| No. 18 | CDR1 | CDR2 | CDR3 |
| Light Chain | QSLLNSGNQKNY | WAS | QNDYSYPLT |
| | (SEQ ID NO: 41) | (SEQ ID NO: 28) | (SEQ ID NO: 29) |
| Heavy Chain | GHTFTSYW | IAPGSGST | ARNLKYGNYGFAY |
| | (SEQ ID NO: 42) | (SEQ ID NO: 43) | (SEQ ID NO: 44) |

### Antibody No. 1

Light Chain: nucleotide sequence (SEQ ID NO:45); amino acid sequence (SEQ ID NO:46)
Heavy Chain: nucleotide sequence (SEQ ID NO:47); amino acid sequence (SEQ ID NO:48)

### Antibody No. 6

Light Chain: nucleotide sequence (SEQ ID NO:49); amino acid sequence (SEQ ID NO:50)
Heavy Chain: nucleotide sequence (SEQ ID NO:51); amino acid sequence (SEQ ID NO:52)

### Antibody No. 7

Light Chain: nucleotide sequence (SEQ ID NO:53); amino acid sequence (SEQ ID NO:54)
Heavy Chain: nucleotide sequence (SEQ ID NO:55); amino acid sequence (SEQ ID NO:56)

### Antibody No. 8

Light Chain: nucleotide sequence (SEQ ID NO:57); amino acid sequence (SEQ ID NO:58)
Heavy Chain: nucleotide sequence (SEQ ID NO:59); amino acid sequence (SEQ ID NO:60)

### Antibody No. 9

Light Chain: nucleotide sequence (SEQ ID NO:61); amino acid sequence (SEQ ID NO:62)
Heavy Chain: nucleotide sequence (SEQ ID NO:63); amino acid sequence (SEQ ID NO:64)

### Antibody No. 11

Light Chain: nucleotide sequence (SEQ ID NO:65); amino acid sequence (SEQ ID NO:66)
Heavy Chain: nucleotide sequence (SEQ ID NO:67); amino acid sequence (SEQ ID NO:68)

### Antibody No. 12

Light Chain: nucleotide sequence (SEQ ID NO:69); amino acid sequence (SEQ ID NO:70)
Heavy Chain: nucleotide sequence (SEQ ID NO:71); amino acid sequence (SEQ ID NO:72)

### Antibody No. 14

Light Chain: nucleotide sequence (SEQ ID NO:73); amino acid sequence (SEQ ID NO:74)
Heavy Chain: nucleotide sequence (SEQ ID NO:75); amino acid sequence (SEQ ID NO:76)

### Antibody No. 15

Light Chain: nucleotide sequence (SEQ ID NO:77); amino acid sequence (SEQ ID NO:78)
Heavy Chain: nucleotide sequence (SEQ ID NO:79); amino acid sequence (SEQ ID NO:80)

### Antibody No. 18

Light Chain: nucleotide sequence (SEQ ID NO:81); amino acid sequence (SEQ ID NO:82)
Heavy Chain: nucleotide sequence (SEQ ID NO:83); amino acid sequence (SEQ ID NO:84)

### Example 5

### Binding to and Killing of Cancer Cells by Anti-PSMA Antibodies

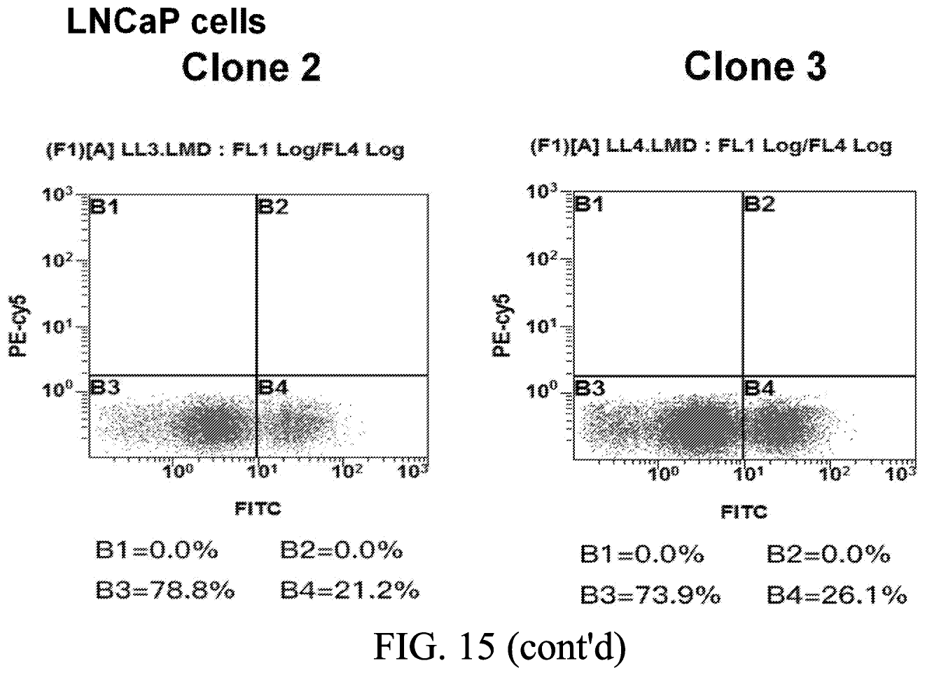

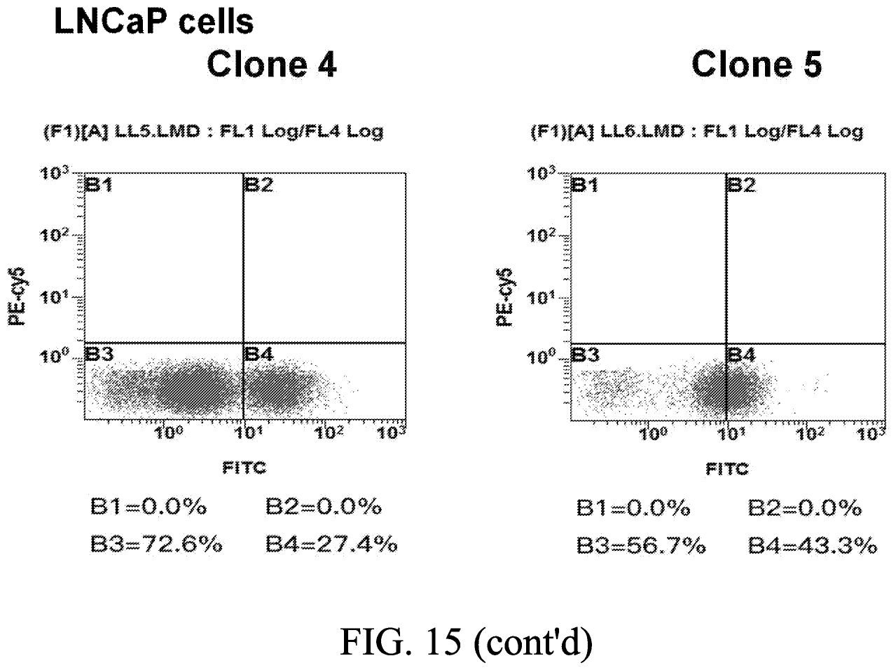

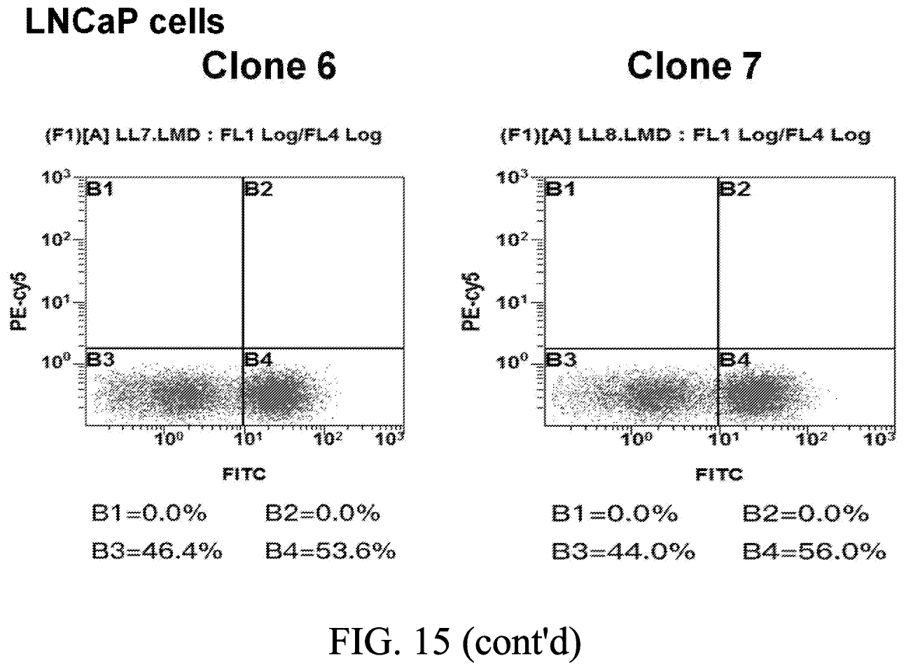

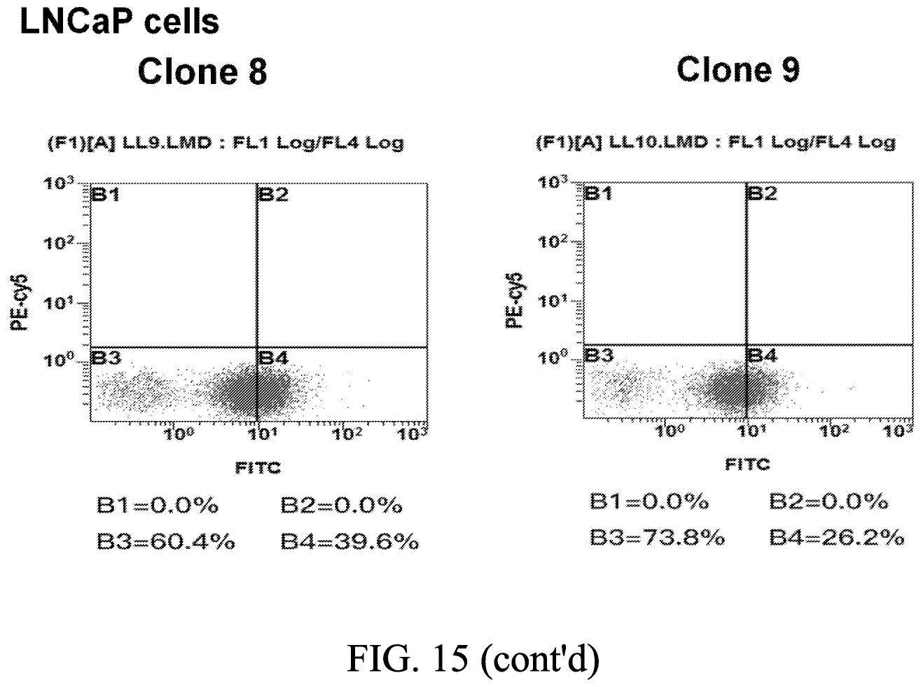

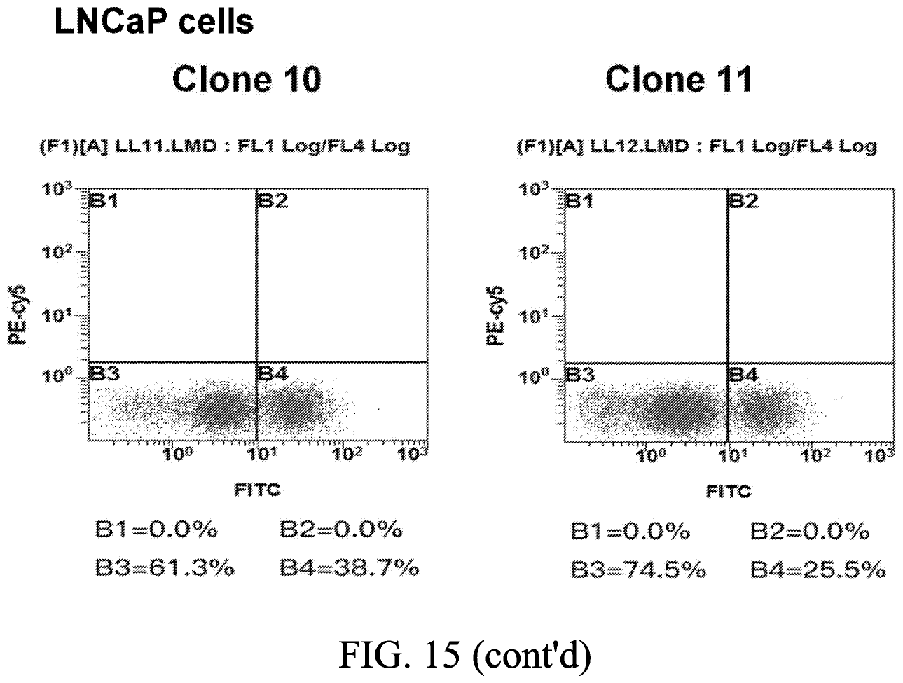

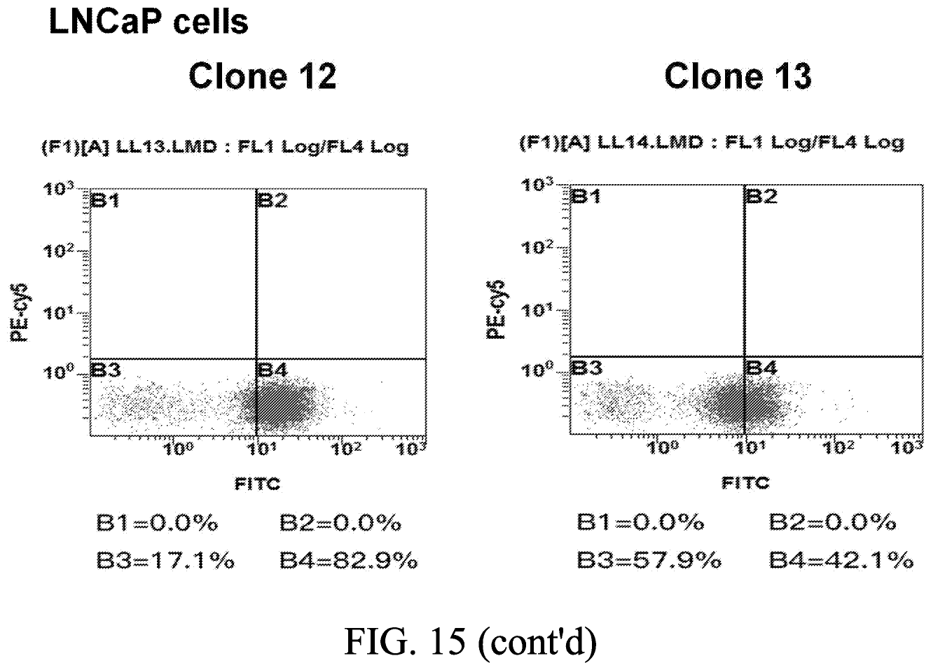

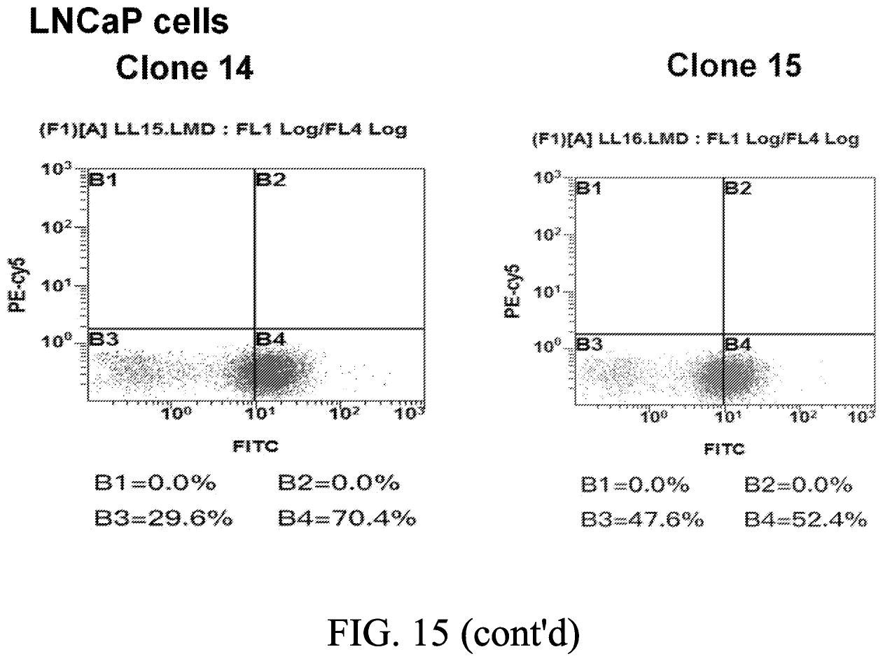

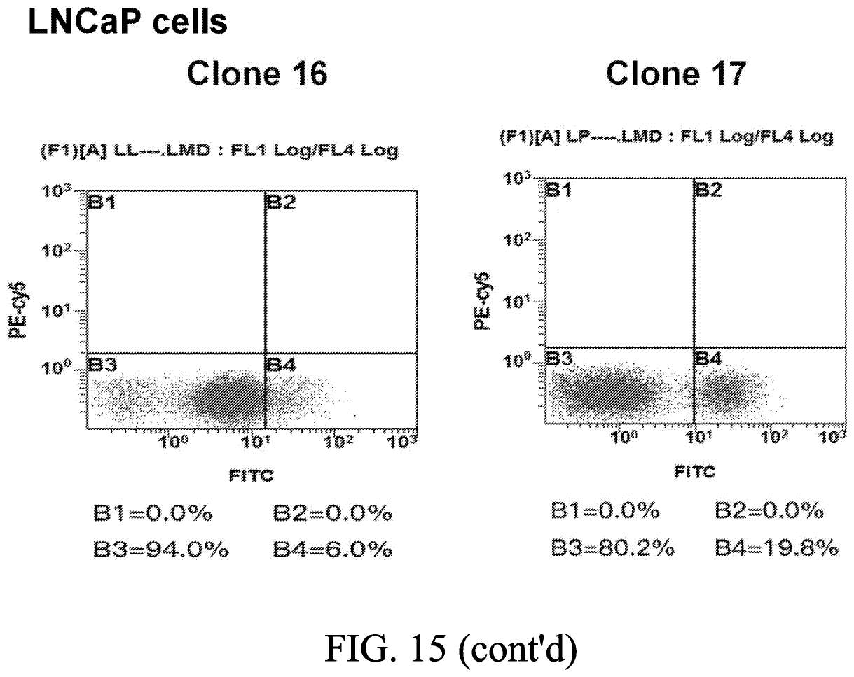

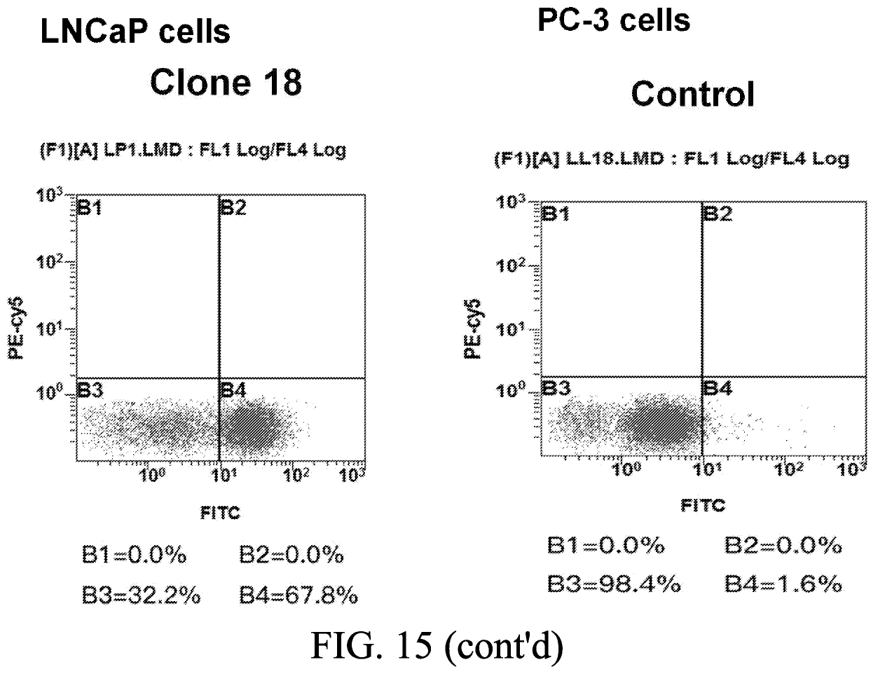

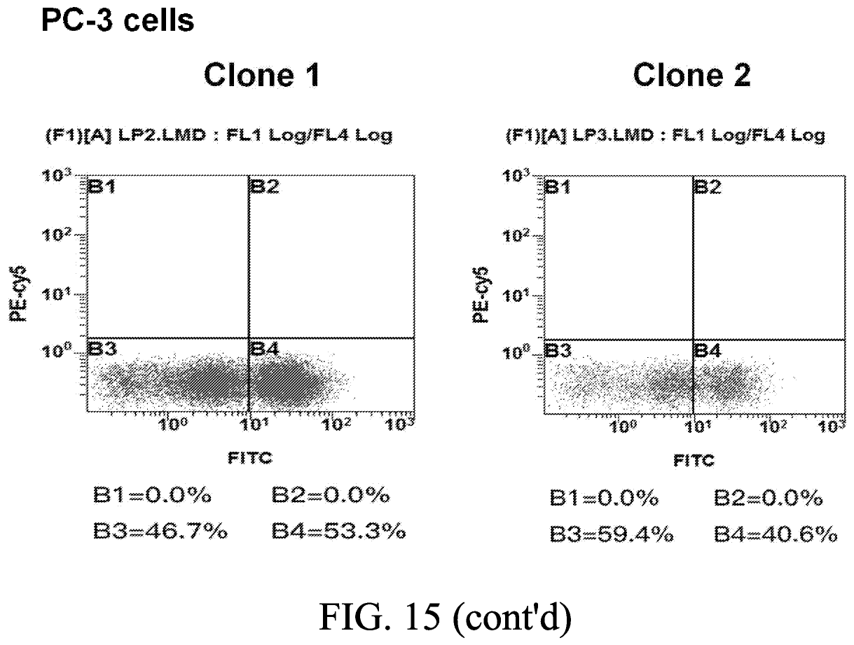

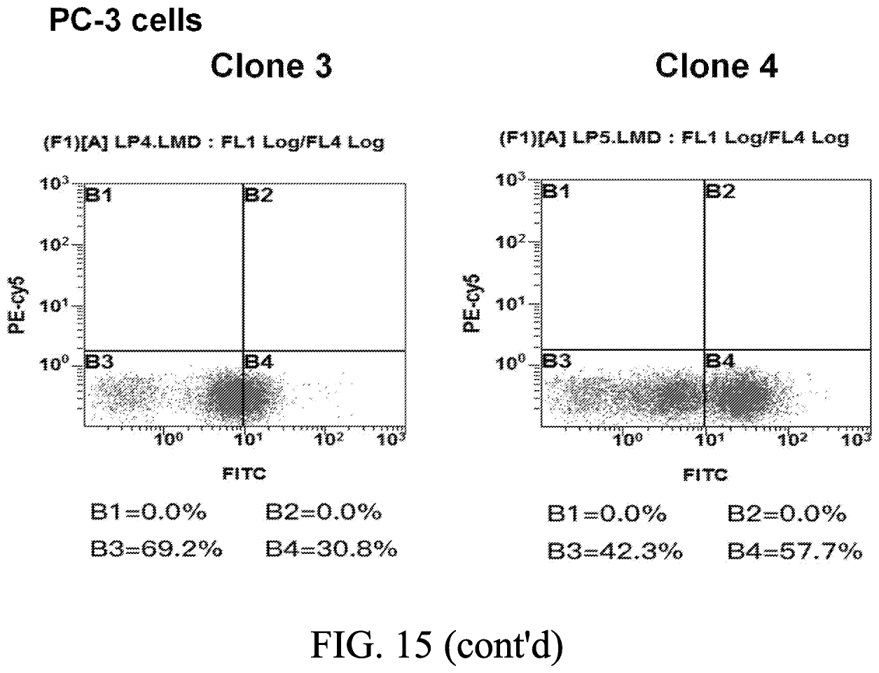

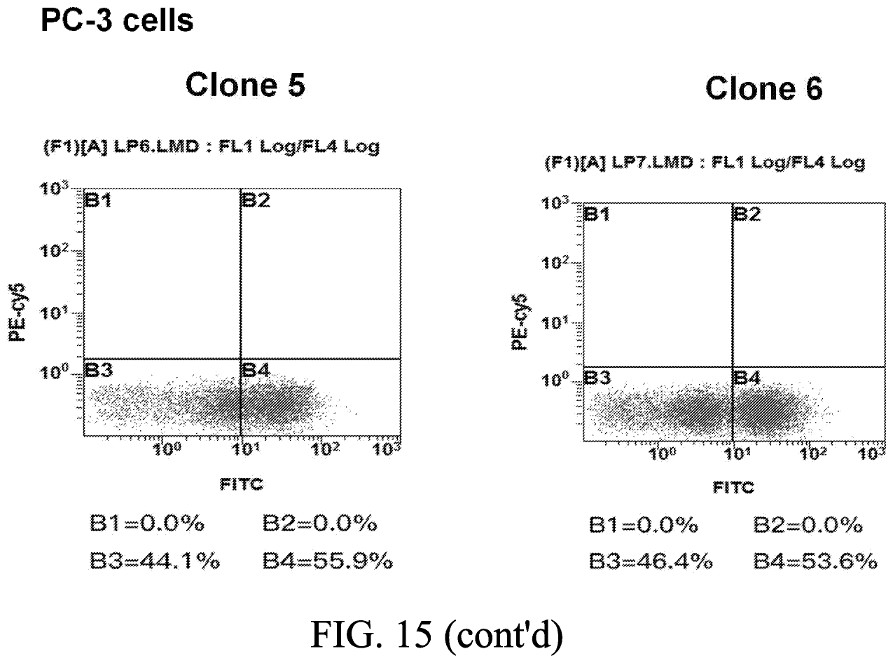

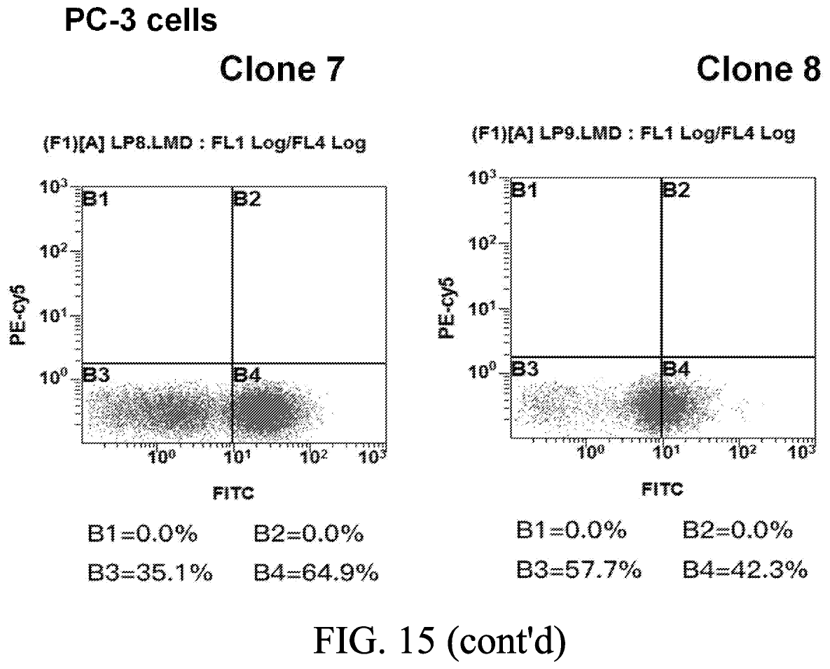

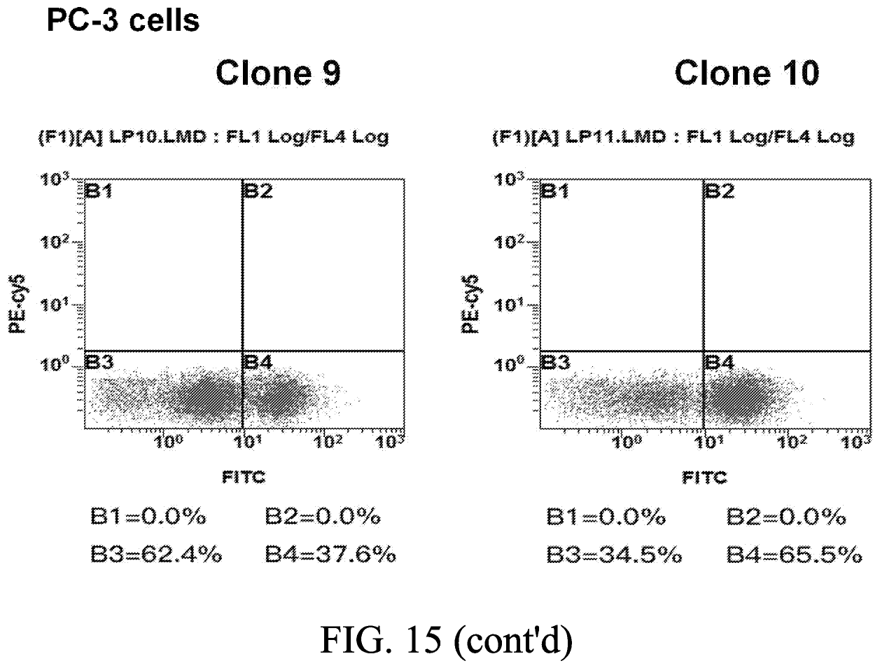

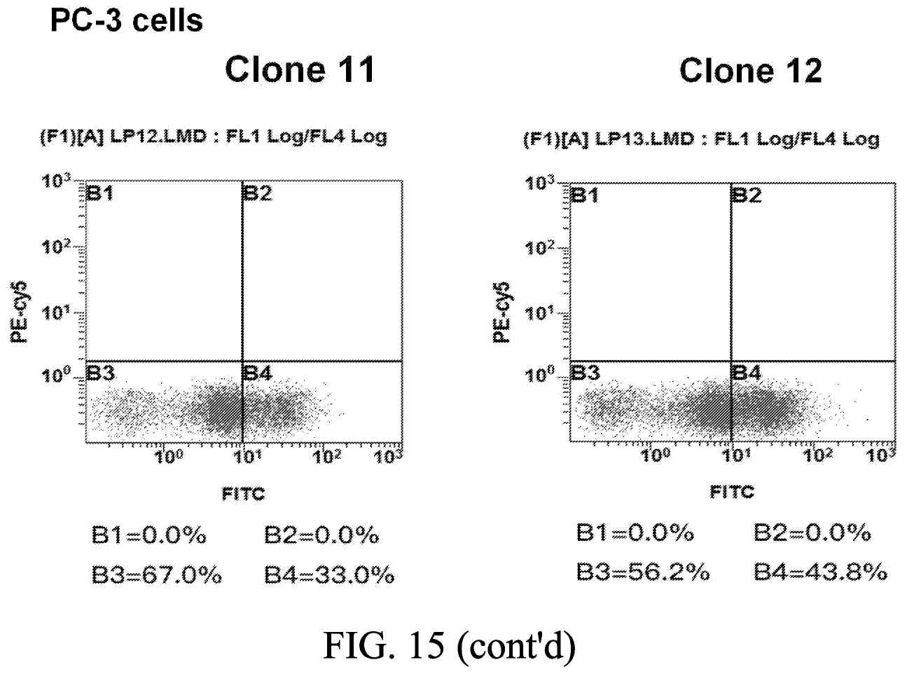

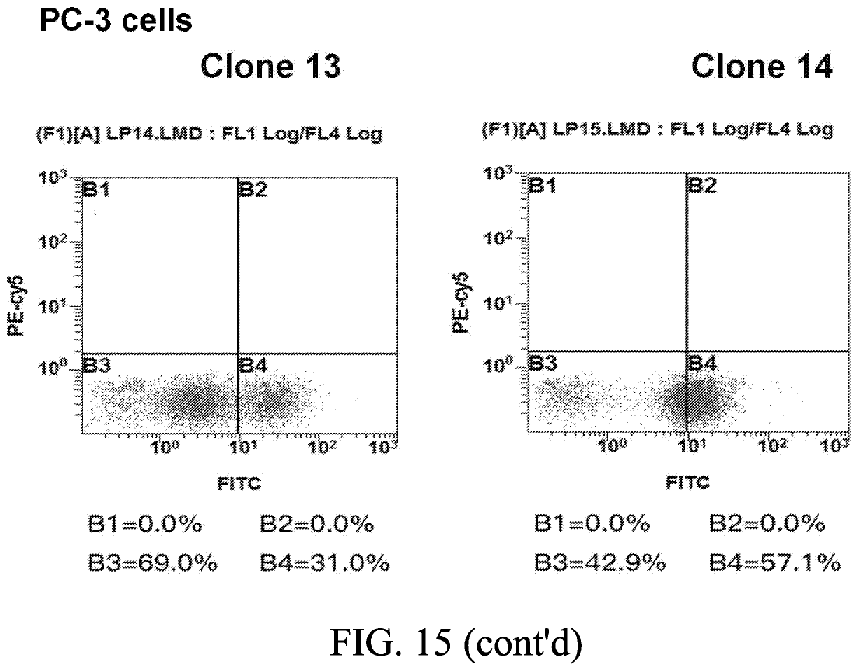

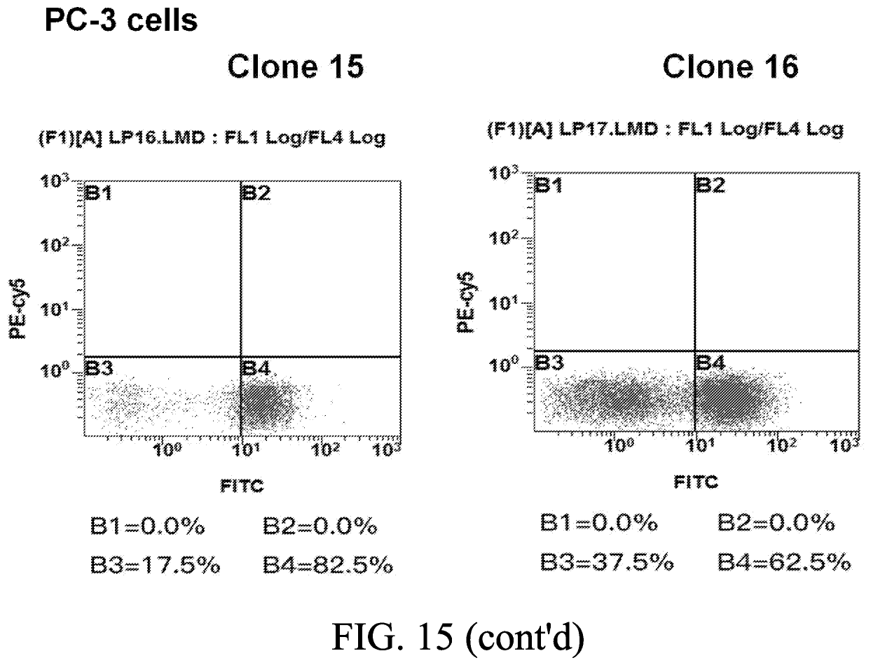

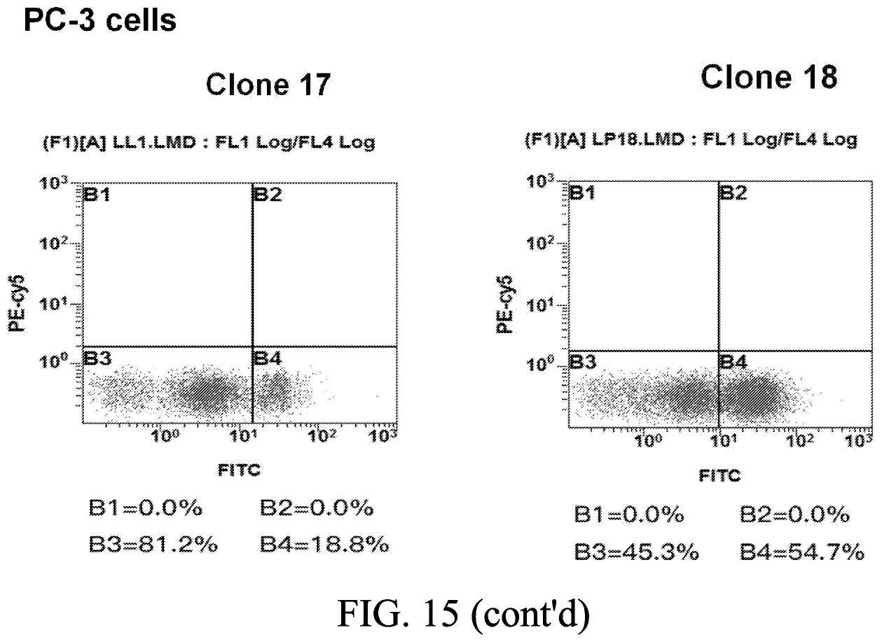

FIG. 15 shows results of binding of the PSMA-McAb derived from the 18 clones to prostate cancer cells identified by flow cytometry.

The PSMA-McAb derived from the 18 clones was reacted with PSMA-expressing prostate cancer LNCaP cells and PC-3 cells respectively to verify whether the PSMA-McAb can specifically bind to the PSMA. The verification steps were as follows:
A. The LNCap and PC-3 cells were separately cultured to a logarithmic growth phase in RPMI1640 culture media (GIBCO, USA) containing 10% fetal bovine serum.
B. The cells were harvested and the cell concentration was adjusted to 5×10⁶-1×10⁷/ml. 40 μL of the cell suspension was well mixed with 5-50 μL of the PSMA-McAb, and stood for 30 min at 4° C.
C. After washing the cells twice with a washing solution, 50 μL of FITC-labeled goat anti-mouse IgG (IgG1, IgG2a, IgG2b, or IgG3, BD Biosciences, USA) was added to the cells, and the mixture was kept at 4° C. for 30 minutes.
D. After the cells were washing twice with a washing solution and 1 ml of a fixative was added, the samples were tested by flow cytometry. Each sample was tested 3 times.

The test results were as shown in FIG. 15. The PSMA-McAb derived from the 18 clones had significant differences in the binding ability to PSMA. For the LNCap cells, the binding ability to PSMA ranged from 6.0% to 82.9%. Among them, clones 1, 6, 7, 12, 14, 15, and 18 had a strong binding ability (p<0.05). For the PC-3 cells, the binding ability to PSMA ranged from 18.7% to 82.5%. Among them, clones 1, 4, 5, 6, 7, 10, 14, 15, 16, and 18 had a strong binding ability (p<0.05). Clones 1, 6, 7, 14, 15, and 18 (p<0.05) had a strong PSMA binding ability to both of the two kinds of cells.

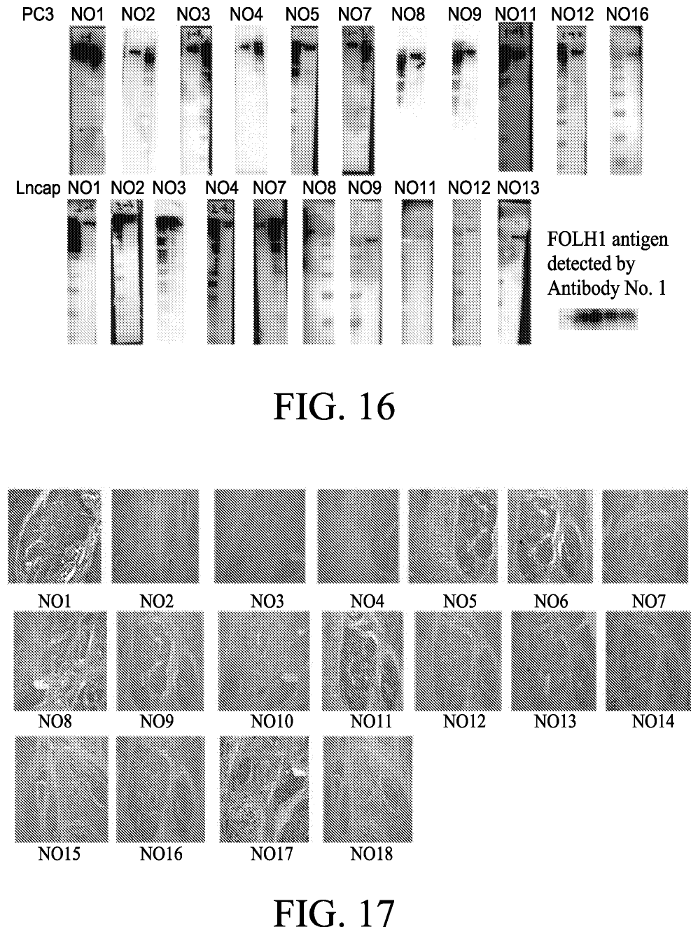

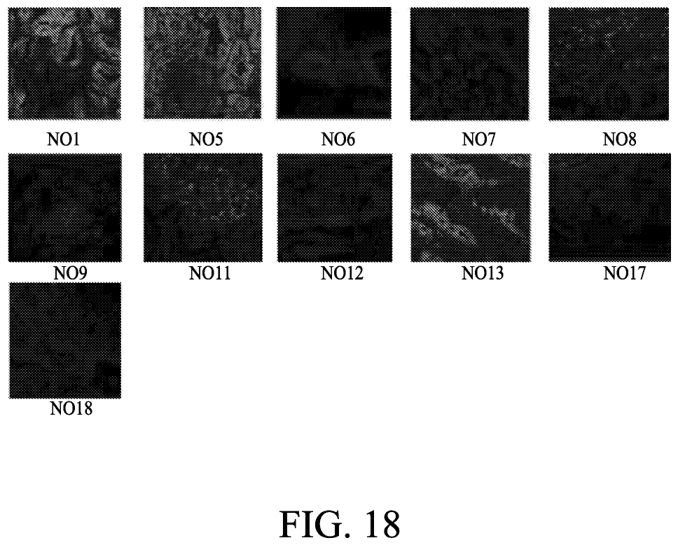

In order to further verify the specific binding ability of the PSMA-McAb derived from the 18 clones to PSMA, the following three experiments were carried out to confirm that the obtained monoclonal antibody was PSMA-McAb that can specifically bind to PSMA.
A. Using the pET-28a-SUMO/PSMA expressed protein containing the protease domain of PSMA as an antigen, immunoblotting verification was performed according to a conventional method of Western Blotting. The second antibody was HRP-labeled goat anti-mouse polyclonal antibody IgG (Sigma-Aldrich, USA). The results were as shown in FIG. 16.
B. A prostate cancer tissue that had been diagnosed as PSAM-positive was used as a test object, which was tested according to a conventional method of immunohistochemistry using the PSMA-McAb derived from the 18 clones. The second antibody was HRP-labeled goat anti-mouse polyclonal antibody IgG (Sigma-Aldrich, USA). The results were as shown in FIG. 17.
C. A prostate cancer tissue that had been diagnosed as PSAM-positive was used as a test object, which was tested according to a conventional method of immunohistochemistry using the PSMA-McAb derived from the 18 clones. The second antibody was Fluorescein-labeled goat anti-mouse polyclonal antibody IgG (Sigma-Aldrich, USA). The results were as shown in FIG. 18.

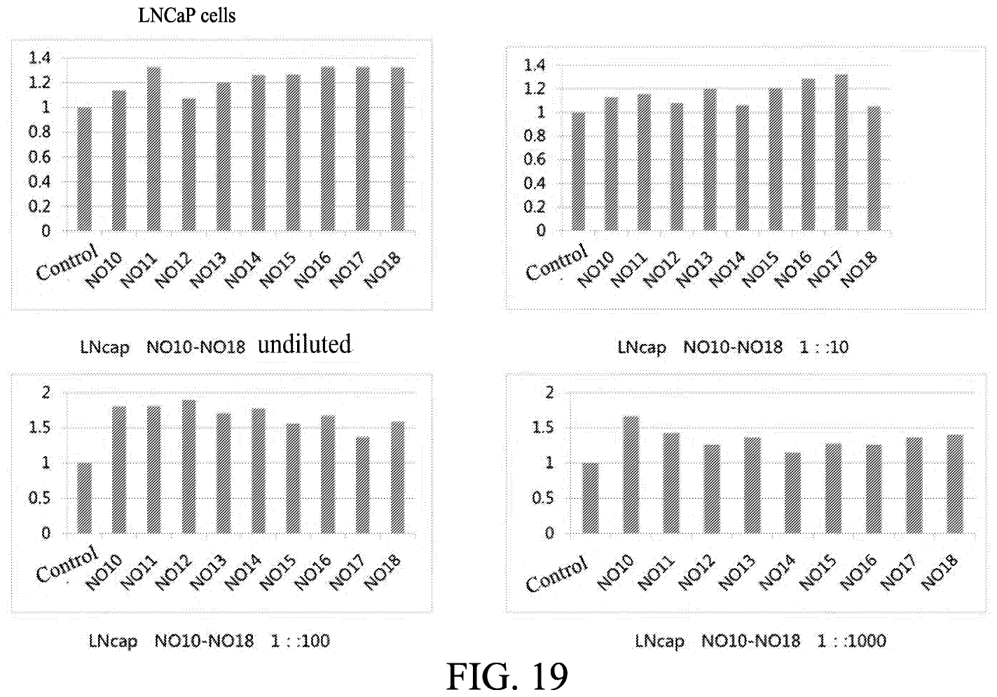

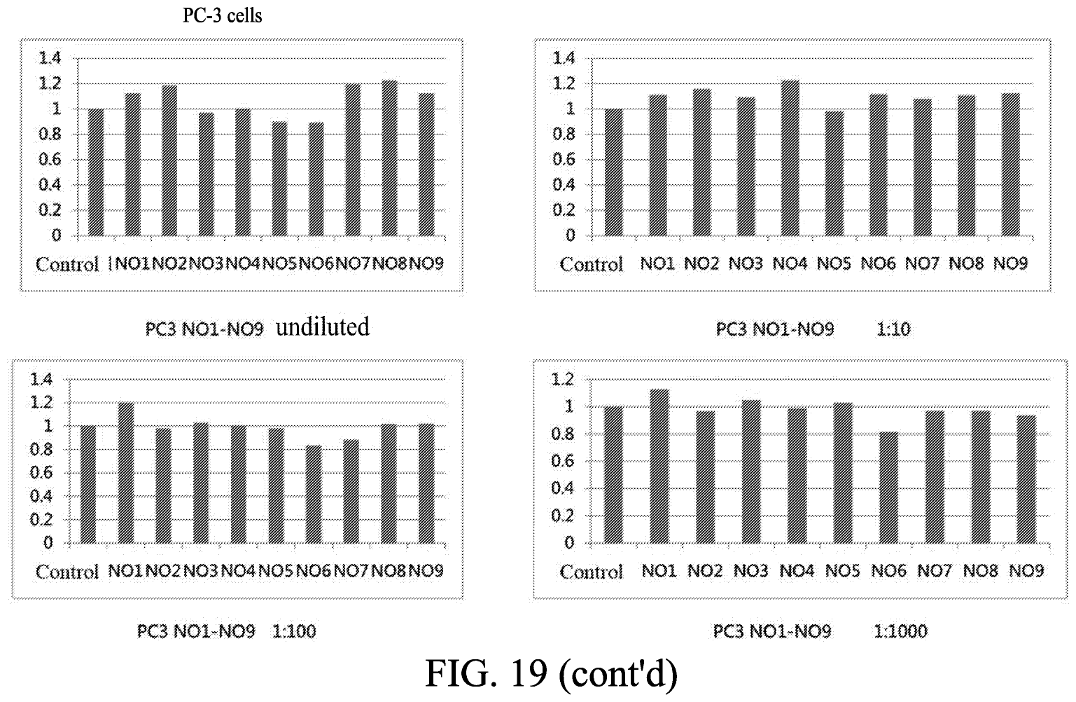

FIG. 19 shows experimental results of inhibition of the proliferation of prostate cancer cells by the PSMA-McAb.

In order to study the ability of the PSMA-McAb from the 18 clones to inhibit the growth of prostate cancer cells, a Cell Counting Kit 8 (WST-8/CCK8) (Abcam, USA) was used according to the manufacturer's instructions. In short, a 4000 cells/100 μL cell suspension was added to a 96-well plates with a clear flat bottom. After culture under conditions of 37° C. and 5% CO₂ for 24 hours, 100 μL of the PSMA-McAb derived from the 18 clones was added (the final concentration was 200 μg/mL). After continual culture for 24 hours, 10 μL of CCK8 was added to each well. 4 hours later, a microplate reader was used to measure absorbance at OD=460 nm.

The cell survival rate was used as a result judgment standard, where the cell survival rate={(As−Ab/(Ac−Ab)}×100%.

Blank control well (Ab): (contained a culture medium and a CCK8 solution, but contained no cells and antibodies)

Control well (Ac): (contained a culture medium, a CCK8 solution, and cells, but contained no antibodies)

Test well (As): (contained a culture medium, a CCK8 solution, cells, and antibodies)

As shown in FIG. 19, when the final concentration of PSMA-McAb from different clones was 200 μg/mL, the degrees of inhibiting the proliferation of prostate cancer LNCap and PC-3 cells varied.

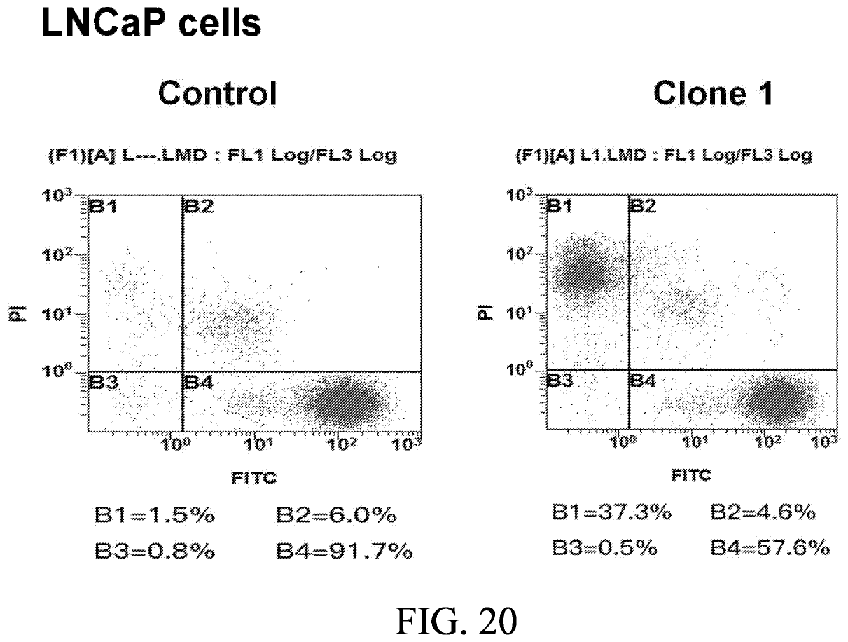

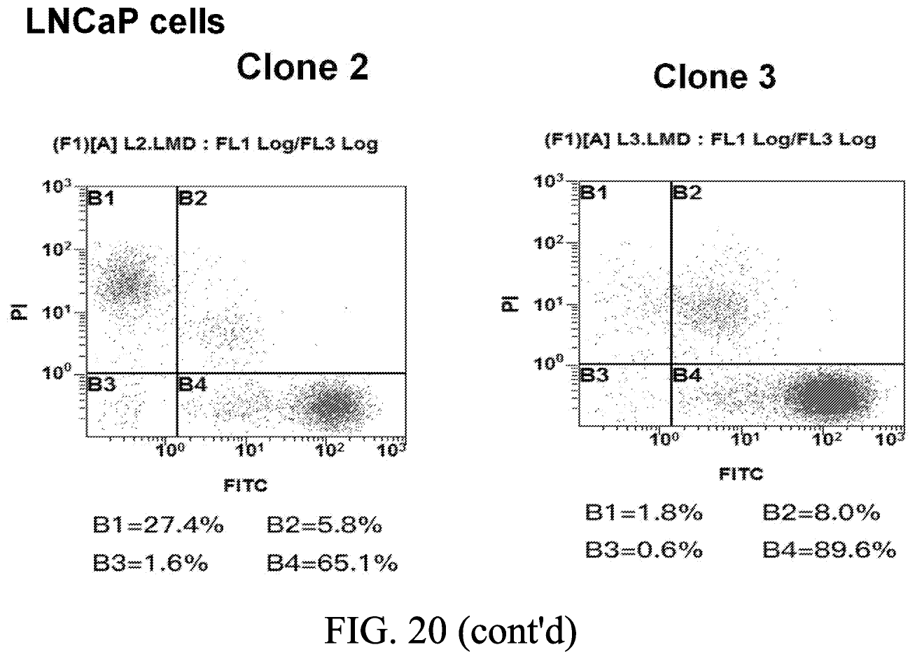

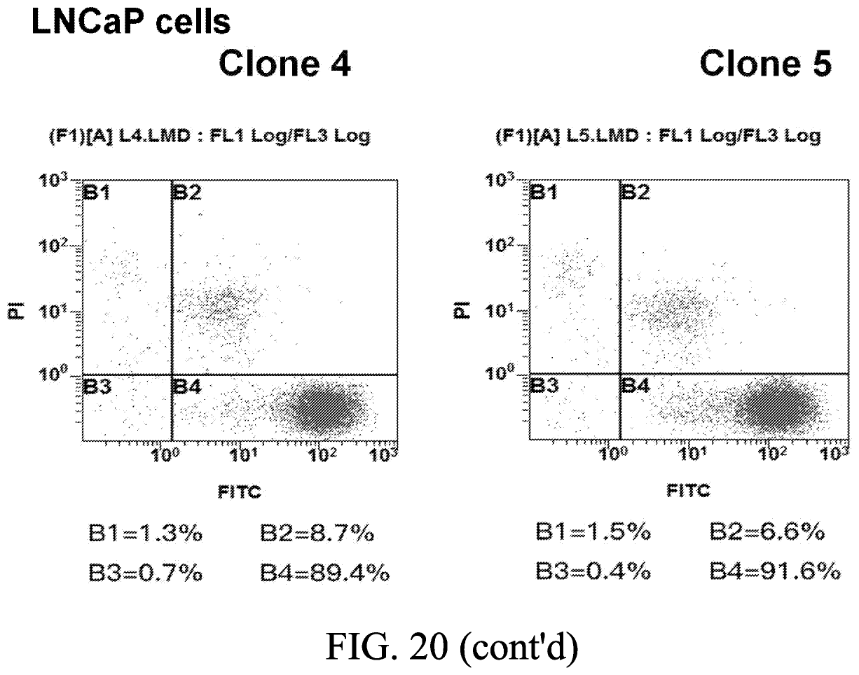

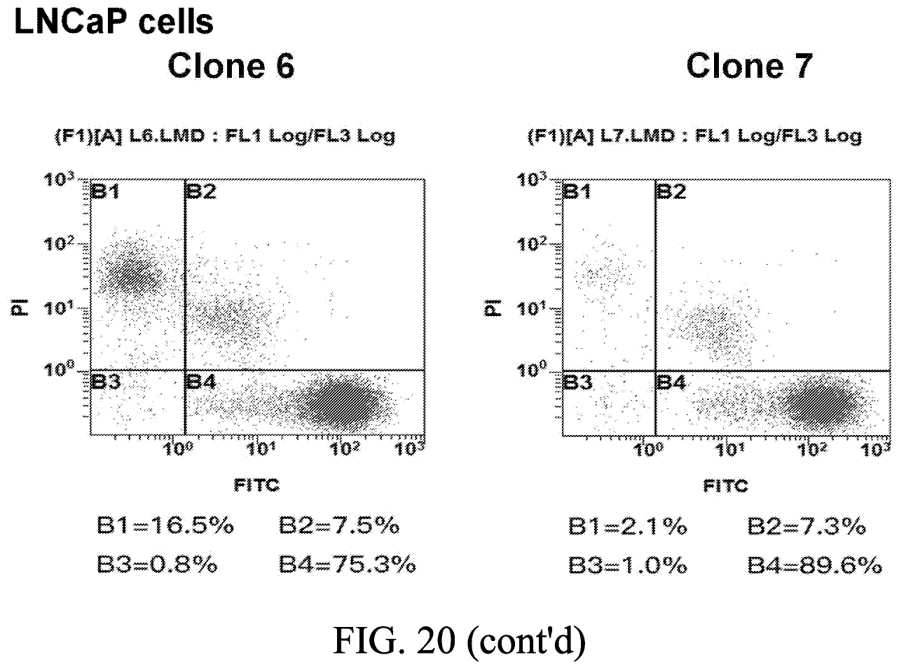

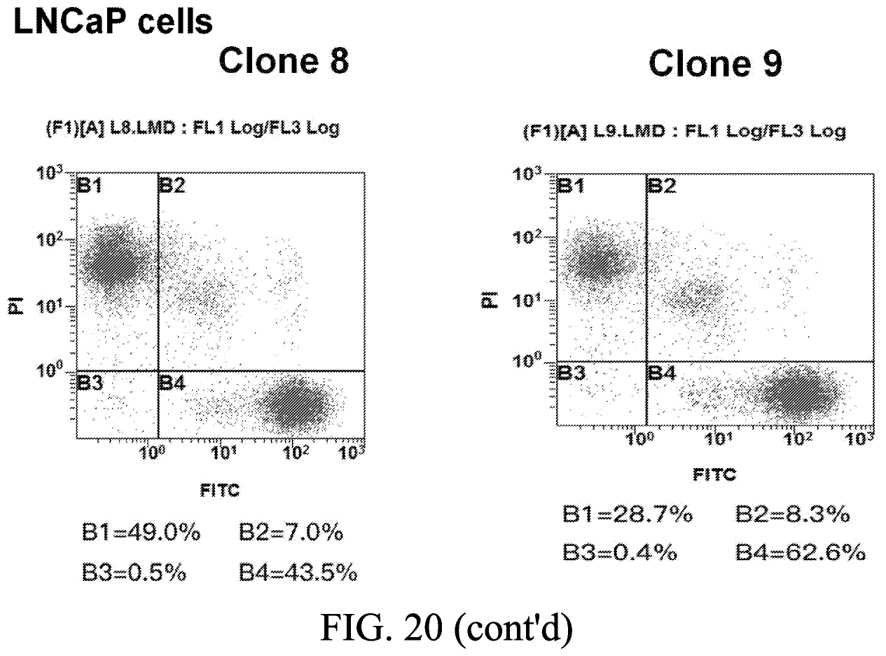

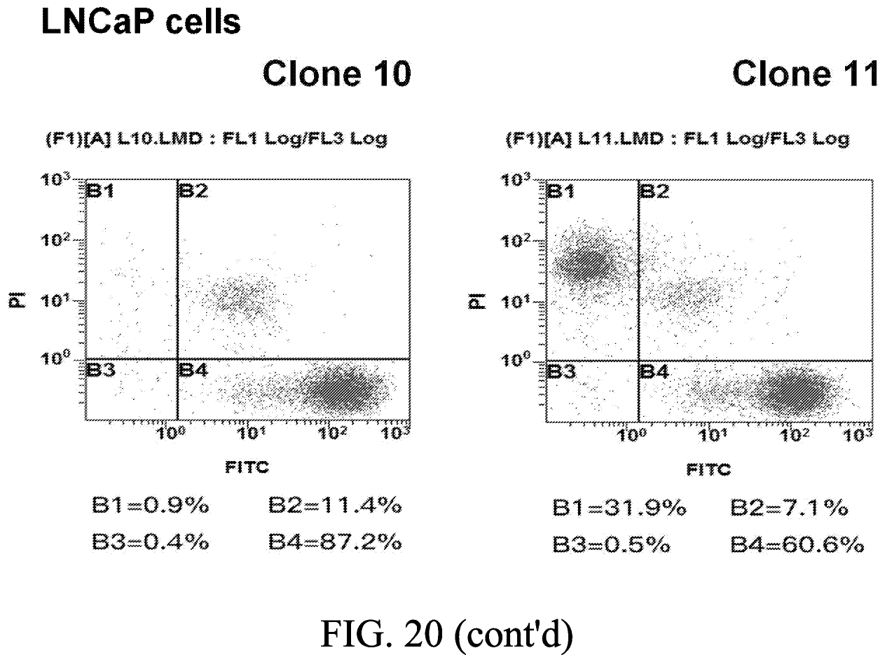

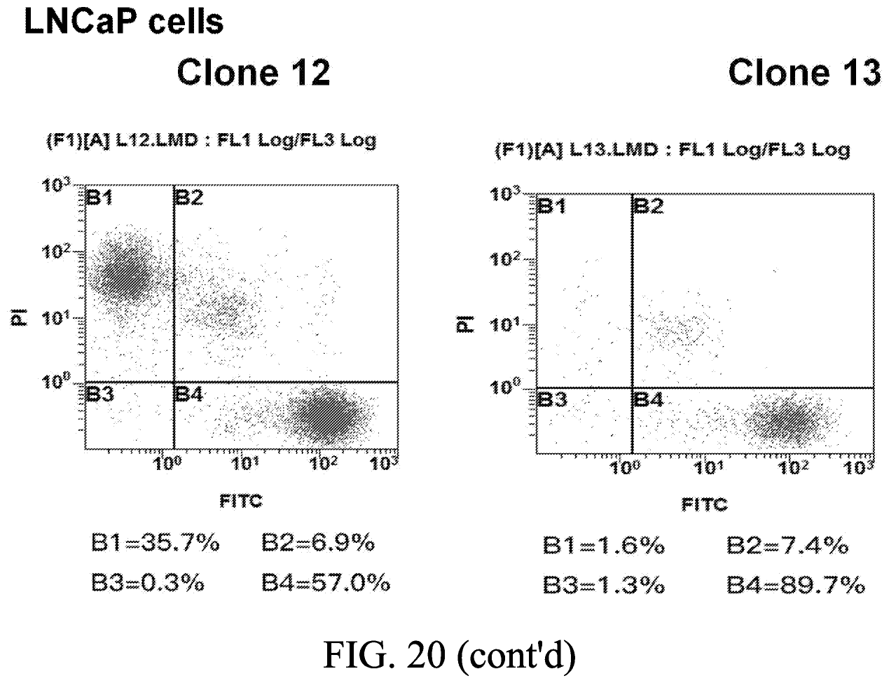

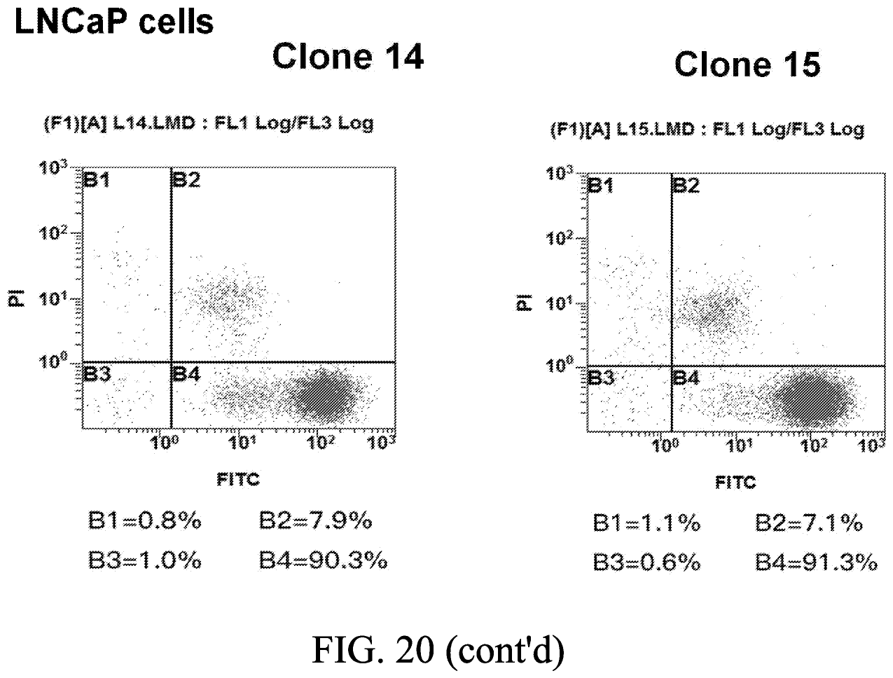

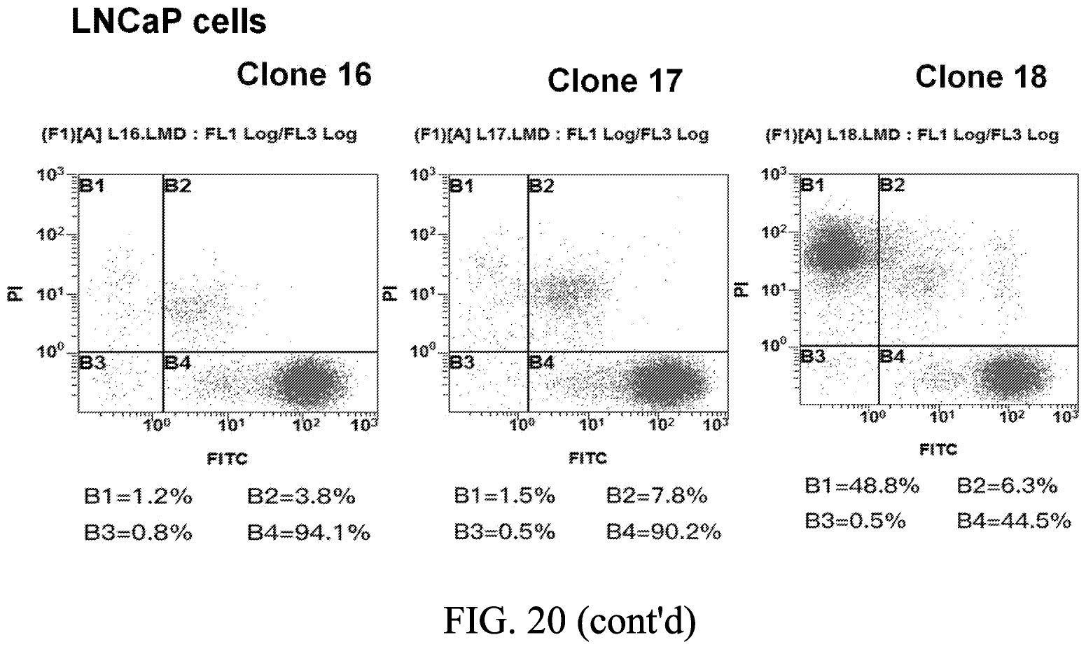

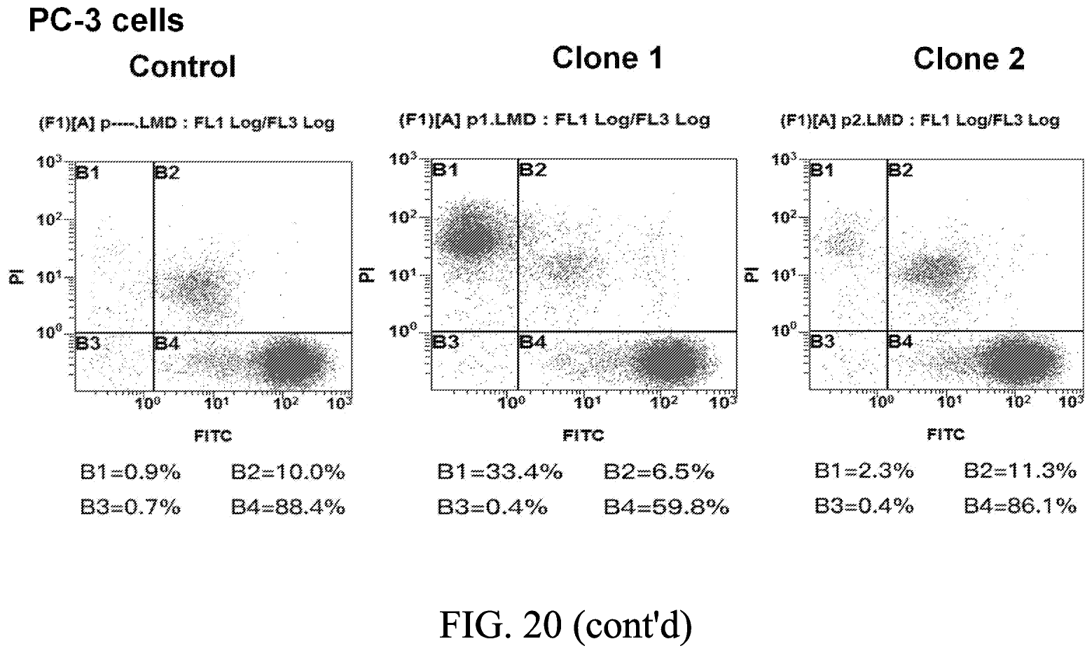

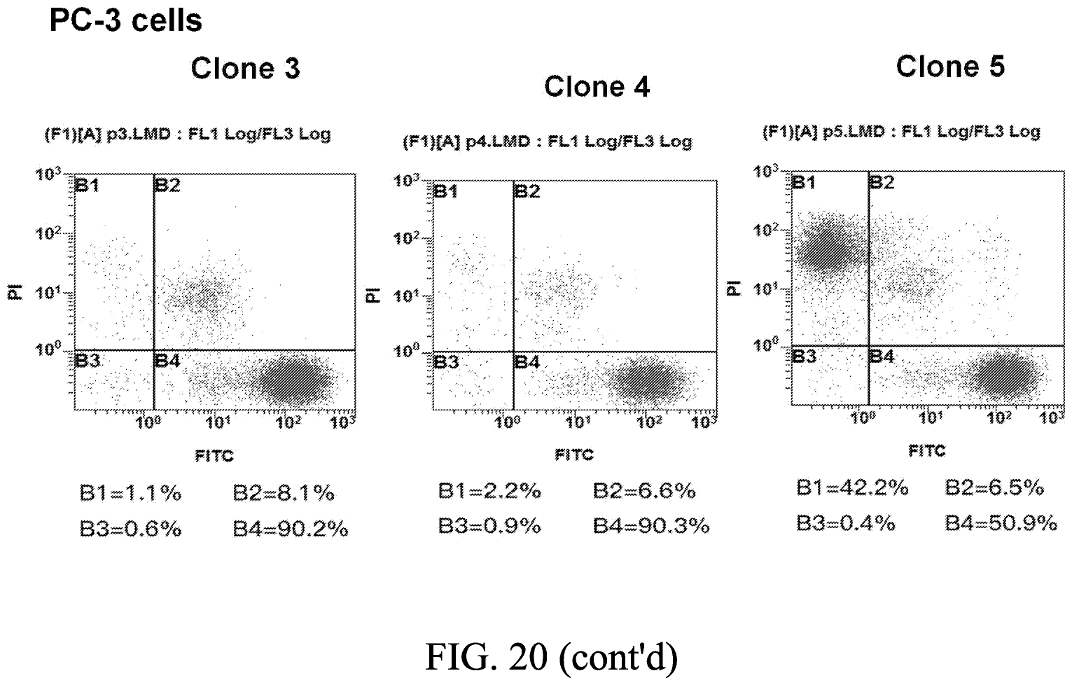

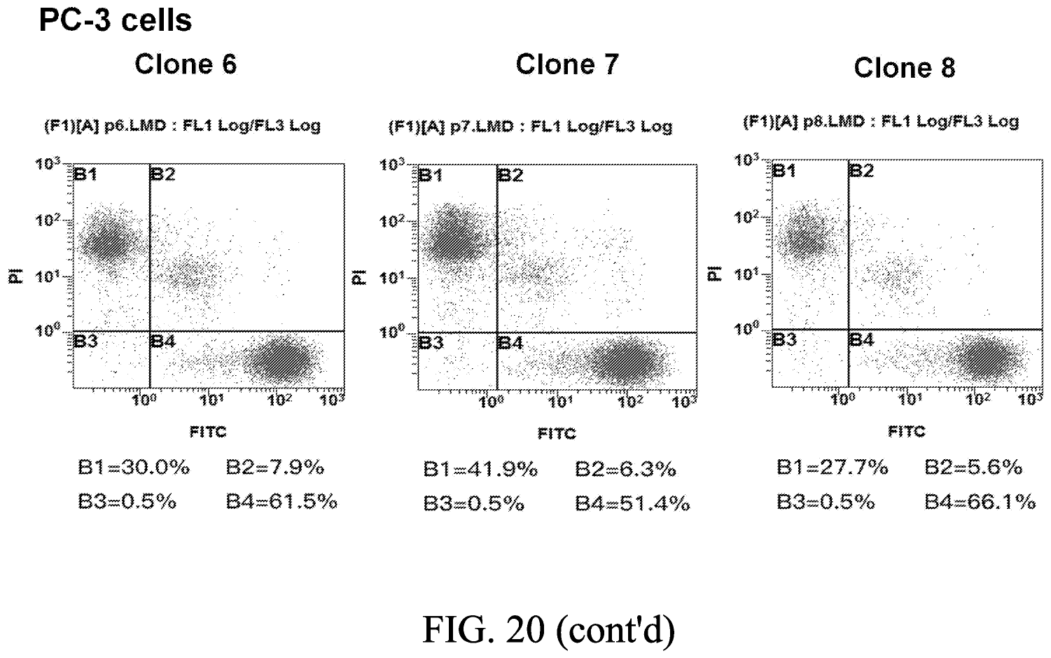

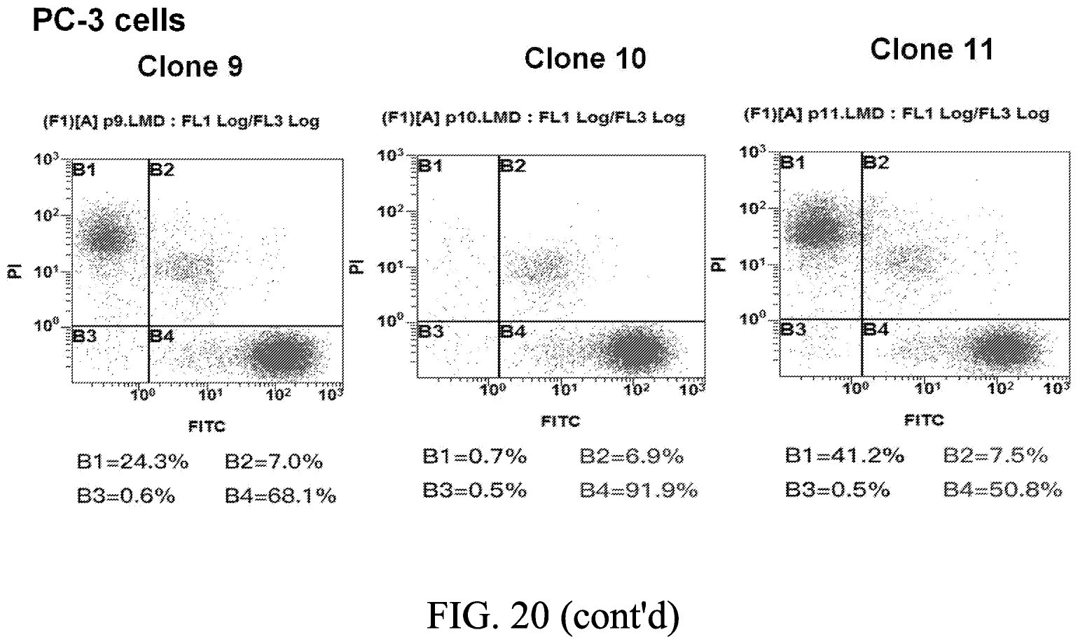

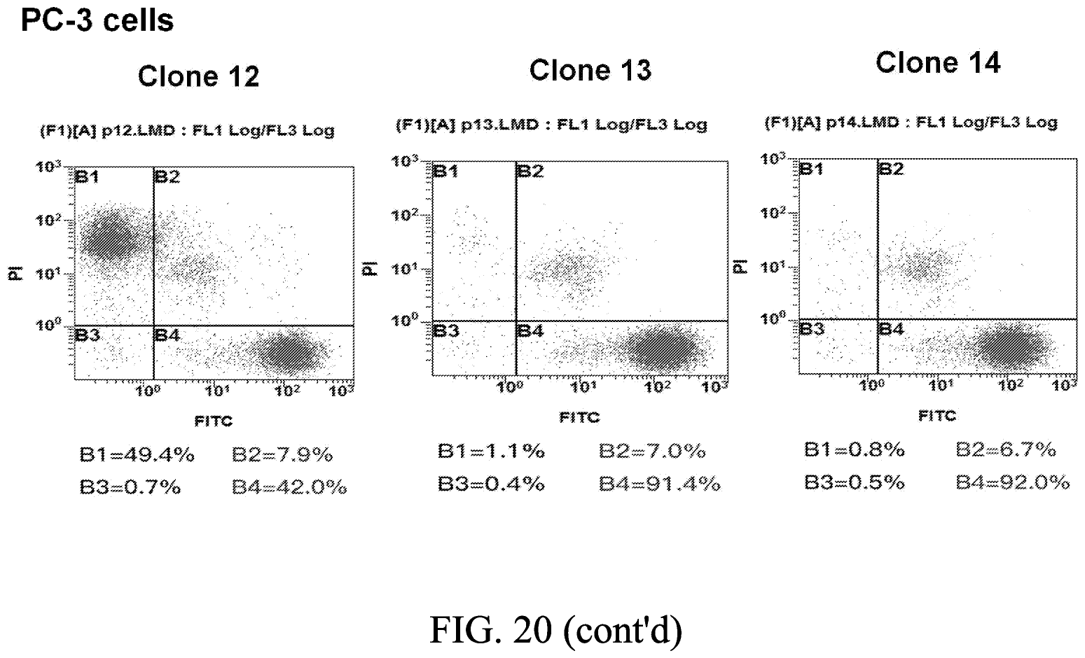

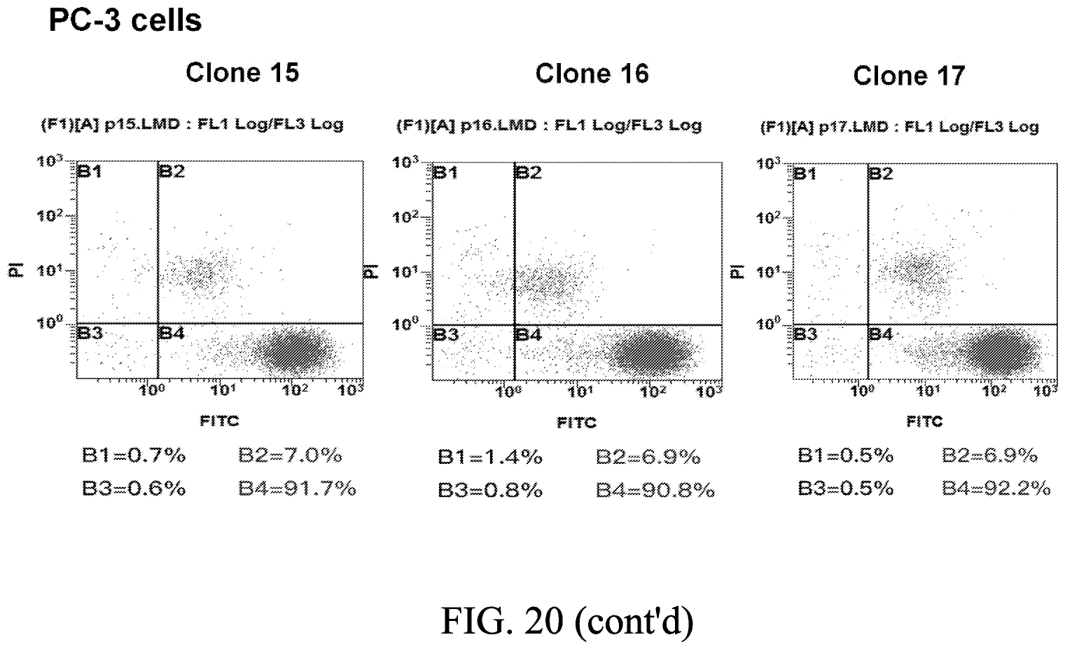

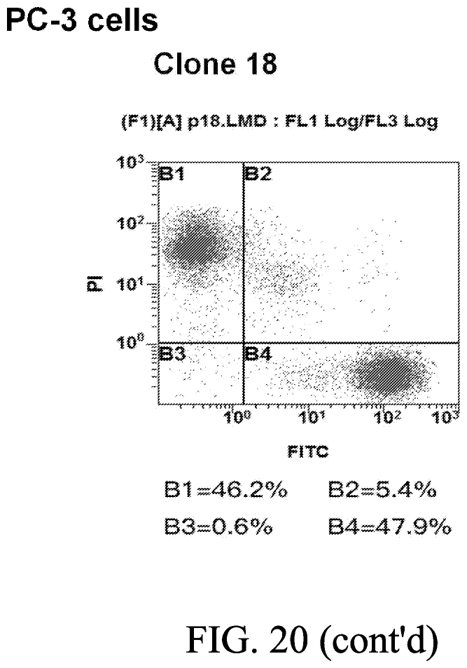

FIG. 20 shows experimental results of antibody-mediated complement-dependent cytotoxicity.

To further confirm that the PSMA-McAb derived from the 18 clones inhibited the growth of prostate cancer cells, a CF SE/PI double staining cytotoxicity test kit (Abcam, USA) was used to conduct an antibody-mediated complement-dependent cytotoxicity experiment according to the instructions provided by the manufacturer, to observe the killing effect of the PSMA-McAb on prostate cancer cells. The experimental steps are briefly described as follows:
A. 50 μL/tube PSMA-McAb (200 μg/mL) was each added to a sterile test tube containing 50 μL of a 4×10⁹/ml cell suspension. The test tubes were kept at 37° C. for 30 min.
B. 50 μL of a complement (uninactivated human serum) was added to each tube. The test tubes were placed in a water bath at 37° C. for 30 min.
C. Each test tube was centrifuged and the supernatant was discarded, and then the test tube was washed twice with PBS (1000 rpm, 10 min).
D. 1:1000 diluted solution A (Calcein-AM, live cell staining) was added to resuspend the cells according to 200 μl/tube. After 30 min at room temperature, each test tube was centrifuged and the supernatant was discarded, and then the test tube was washed twice with PBS (1000 rpm, 10 min).
E. 1:2000 diluted solution B (PI dead cell stain) was added to resuspend the cells according to 200 μl/tube. After 5 minutes at room temperature, each test tube was centrifuged and the supernatant was discarded, and then the test tube was washed twice with PBS (1000 rpm, 10 min).
F. After the cells were re-suspended with 300 μL of a PBS buffer, the samples were tested by flow cytometry.

The test results are shown in FIG. 20.

There are significant differences in the ability to kill PSMA-positive prostate cancer cells. The killing percentage of LNCaP cells by the PSMA-McAb ranged from 0.8% to 49.0%, and the killing percentage of PC-3 cells by the PSMA-McAb ranged from 0.5% to 49.4%. Compared with the control group, the PSMA-McAb derived from clones 1, 8, 9, 11, 12, and 18 had obvious killing effects on LNCaP cells and PC-3 cells, with the killing percentage ranging from 24.3% to 49.4% (p <0.05). The PSMA-McAb derived from clone 2 had a significant killing effect only on LNCaP cells (27.4% (p<0.05). The PSMA-McAb derived from clones 5, 6, and 7 had a significant killing effect only on PC-3 cells (42.2%, 30.0%, 41.9%) (p<0.05).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 63/111,537, filed Nov. 9, 2020, which application is hereby incorporated by reference in its entirety.

1. An isolated antibody or an antigen-binding fragment thereof that binds to PSMA, wherein the antibody or antigen-binding fragment thereof comprises a light chain variable region (VL) and a heavy chain variable region (VH), wherein
(a) the VL comprises a heavy chain CDR1 (VL-CDR1), heavy chain CDR2 (VL-CDR2), and heavy chain CDR3 (VL-CDR3) comprising the amino acid sequences of SEQ ID NO:1, SEQ ID NO:2, and SEQ ID NO:3, respectively; and the VH comprises a light chain CDR1 (VH-CDR1), light chain CDR2 (VH-CDR2), and light chain CDR3 (VH-CDR3) comprising the amino acid sequences of SEQ ID NO:4, SEQ ID NO:5, and SEQ ID NO:6, respectively;
(b) the VL comprises a heavy chain CDR1 (VL-CDR1), heavy chain CDR2 (VL-CDR2), and heavy chain CDR3 (VL-CDR3) comprising the amino acid sequences of SEQ ID NO:7, SEQ ID NO:2, and SEQ ID NO:8, respectively; and the VH comprises a light chain CDR1 (VH-CDR1), light chain CDR2 (VH-CDR2), and light chain CDR3 (VH-CDR3) comprising the amino acid sequences of SEQ ID NO:9, SEQ ID NO:10, and SEQ ID NO:11, respectively;
(c) the VL comprises a heavy chain CDR1 (VL-CDR1), heavy chain CDR2 (VL-CDR2), and heavy chain CDR3 (VL-CDR3) comprising the amino acid sequences of SEQ ID NO:1, SEQ ID NO:2, and SEQ ID NO:3, respectively; and the VH comprises a light chain CDR1 (VH-CDR1), light chain CDR2 (VH-CDR2), and light chain CDR3 (VH-CDR3) comprising the amino acid sequences of SEQ ID NO:12, SEQ ID NO:13, and SEQ ID NO:14, respectively;
(d) the VL comprises a heavy chain CDR1 (VL-CDR1), heavy chain CDR2 (VL-CDR2), and heavy chain CDR3 (VL-CDR3) comprising the amino acid sequences of SEQ ID NO:1, SEQ ID NO:2, and SEQ ID NO:3, respectively; and the VH comprises a light chain CDR1 (VH-CDR1), light chain CDR2 (VH-CDR2), and light chain CDR3 (VH-CDR3) comprising the amino acid sequences of SEQ ID NO:15, SEQ ID NO:16, and SEQ ID NO:17, respectively;
(e) the VL comprises a heavy chain CDR1 (VL-CDR1), heavy chain CDR2 (VL-CDR2), and heavy chain CDR3 (VL-CDR3) comprising the amino acid sequences of SEQ ID NO:1, SEQ ID NO:2, and SEQ ID NO:3, respectively; and the VH comprises a light chain CDR1 (VH-CDR1), light chain CDR2 (VH-CDR2), and light chain CDR3 (VH-CDR3) comprising the amino acid sequences of SEQ ID NO:18, SEQ ID NO:19, and SEQ ID NO:20, respectively;
(f) the VL comprises a heavy chain CDR1 (VL-CDR1), heavy chain CDR2 (VL-CDR2), and heavy chain CDR3 (VL-CDR3) comprising the amino acid sequences of SEQ ID NO:21, SEQ ID NO:22, and SEQ ID NO:23, respectively; and the VH comprises a light chain CDR1 (VH-CDR1), light chain CDR2 (VH-CDR2), and light chain CDR3 (VH-CDR3) comprising the amino acid sequences of SEQ ID NO:24, SEQ ID NO:25, and SEQ ID NO:26, respectively;
(g) the VL comprises a heavy chain CDR1 (VL-CDR1), heavy chain CDR2 (VL-CDR2), and heavy chain CDR3 (VL-CDR3) comprising the amino acid sequences of SEQ ID NO:27, SEQ ID NO:28, and SEQ ID NO:29, respectively; and the VH comprises a light chain CDR1 (VH-CDR1), light chain CDR2 (VH-CDR2), and light chain CDR3 (VH-CDR3) comprising the amino acid sequences of SEQ ID NO:30, SEQ ID NO:31, and SEQ ID NO:32, respectively;
(h) the VL comprises a heavy chain CDR1 (VL-CDR1), heavy chain CDR2 (VL-CDR2), and heavy chain CDR3 (VL-CDR3) comprising the amino acid sequences of SEQ ID NO:33, SEQ ID NO:22, and SEQ ID NO:34, respectively; and the VH comprises a light chain CDR1 (VH-CDR1), light chain CDR2 (VH-CDR2), and light chain CDR3 (VH-CDR3) comprising the amino acid sequences of SEQ ID NO:35, SEQ ID NO:36, and SEQ ID NO:37, respectively;
(i) the VL comprises a heavy chain CDR1 (VL-CDR1), heavy chain CDR2 (VL-CDR2), and heavy chain CDR3 (VL-CDR3) comprising the amino acid sequences of SEQ ID NO:1, SEQ ID NO:2, and SEQ ID NO:3, respectively; and the VH comprises a light chain CDR1 (VH-CDR1), light chain CDR2 (VH-CDR2), and light chain CDR3 (VH-CDR3) comprising the amino acid sequences of SEQ ID NO:38, SEQ ID NO:39, and SEQ ID NO:40, respectively; or
(j) the VL comprises a heavy chain CDR1 (VL-CDR1), heavy chain CDR2 (VH-CDR2), and heavy chain CDR3 (VL-CDR3) comprising the amino acid sequences of SEQ ID NO:41, SEQ ID NO:28, and SEQ ID NO:29, respectively; and the VH comprises a light chain CDR1 (VH-CDR1), light chain CDR2 (VH-CDR2), and light chain CDR3 (VH-CDR3) comprising the amino acid sequences of SEQ ID NO:42, SEQ ID NO:43, and SEQ ID NO:44, respectively.

2. The antibody or antigen binding fragment thereof of claim 1, wherein
(a) the VH comprises an amino acid sequence that has at least 90% identity with the amino acid sequence of SEQ ID NO:48 and the VL comprises an amino acid sequence that has at least 90% identity with the amino acid sequence of SEQ ID NO:46;
(b) the VH comprises an amino acid sequence that has at least 90% identity with the amino acid sequence of SEQ ID NO:52, and the VL comprises an amino acid sequence that has at least 90% identity with the amino acid sequence of SEQ ID NO:50;
(c) the VH comprises an amino acid sequence that has at least 90% identity with the amino acid sequence of SEQ ID NO:56, and the VL comprises an amino acid sequence that has at least 90% identity with the amino acid sequence of SEQ ID NO:54;
(d) the VH comprises an amino acid sequence that has at least 90% identity with the amino acid sequence of SEQ ID NO:60, and the VL comprises an amino acid sequence that has at least 90% identity with the amino acid sequence of SEQ ID NO:58;
(e) the VH comprises an amino acid sequence that has at least 90% identity with the amino acid sequence of SEQ ID NO:64, and the VL comprises an amino acid sequence that has at least 90% identity with the amino acid sequence of SEQ ID NO:62;
(f) the VH comprises an amino acid sequence that has at least 90% identity with the amino acid sequence of SEQ ID NO:68, and the VL comprises an amino acid sequence that has at least 90% identity with the amino acid sequence of SEQ ID NO:66;
(g) the VH comprises an amino acid sequence that has at least 90% identity with the amino acid sequence of SEQ ID NO:72, and the VL comprises an amino acid sequence that has at least 90% identity with the amino acid sequence of SEQ ID NO:70;
(h) the VH comprises an amino acid sequence that has at least 90% identity with the amino acid sequence of SEQ ID NO:76, and the VL comprises an amino acid sequence that has at least 90% identity with the amino acid sequence of SEQ ID NO:74;
(i) the VH comprises an amino acid sequence that has at least 90% identity with the amino acid sequence of SEQ ID NO:80, and the VL comprises an amino acid sequence that has at least 90% identity with the amino acid sequence of SEQ ID NO:78; or
(j) the VH comprises an amino acid sequence that has at least 90% identity with the amino acid sequence of SEQ ID NO:84, and the VL comprises an amino acid sequence that has at least 90% identity with the amino acid sequence of SEQ ID NO:82.

3. The antibody or antigen binding fragment thereof of claim 2, wherein
(a) the VH comprises the amino acid sequence of SEQ ID NO:48 and the VL comprises the amino acid sequence of SEQ ID NO:46;
(b) the VH comprises the amino acid sequence of SEQ ID NO:52, and the VL comprises the amino acid sequence of SEQ ID NO:50;
(c) the VH comprises the amino acid sequence of SEQ ID NO:56, and the VL comprises the amino acid sequence of SEQ ID NO:54;
(d) the VH comprises the amino acid sequence of SEQ ID NO:60, and the VL comprises the amino acid sequence of SEQ ID NO:58;
(e) the VH comprises the amino acid sequence of SEQ ID NO:64, and the VL comprises the amino acid sequence of SEQ ID NO:62;
(f) the VH comprises the amino acid sequence of SEQ ID NO:68, and the VL comprises the amino acid sequence of SEQ ID NO:66;
(g) the VH comprises the amino acid sequence of SEQ ID NO:72, and the VL comprises the amino acid sequence of SEQ ID NO:70;
(h) the VH comprises the amino acid sequence of SEQ ID NO:76, and the VL comprises the amino acid sequence of SEQ ID NO:74;
(i) the VH comprises the amino acid sequence of SEQ ID NO:80, and the VL comprises the amino acid sequence of SEQ ID NO:78; or
(j) the VH comprises the amino acid sequence of SEQ ID NO:84, and the VL comprises the amino acid sequence of SEQ ID NO:82.

4. The antibody or antigen binding fragment thereof of claim 1, wherein the antibody comprises a human IgG1, human IgG2, human IgG3, or human IgG4 constant region or a variant thereof.

5. The antibody or antigen binding fragment thereof of claim 1, wherein the antibody is a humanized antibody.

6. The antibody or antigen binding fragment thereof of claim 1, wherein the antibody is glycosylated.

7. The antibody or antigen binding fragment thereof of claim 1, wherein the antibody has a normal effector function.

8. The antibody or antigen binding fragment thereof of claim 1, wherein the antibody has a reduced effector function.

9. A pharmaceutical composition comprising an antibody or antigen-binding fragment thereof of claim 1 and a pharmaceutically acceptable carrier.

10. An isolated nucleic acid that encodes the HC and LC of the antibody or antigen-binding fragment thereof of claim 1.

11. A vector comprising the nucleic acid of claim 10.

12. An isolated host cell comprising the nucleic acid of claim 10.

13. An isolated host cell that expresses the antibody or antigen-binding fragment thereof of claim 1.

14. A mammalian host cell comprising:
(a) a polynucleotide sequence encoding a polypeptide having an amino acid sequence of SEQ ID NO: 48 and a polynucleotide sequence encoding a polypeptide having an amino acid sequence of SEQ ID NO: 46;
(b) a polynucleotide sequence encoding a polypeptide having an amino acid sequence of SEQ ID NO: 52 and a polynucleotide sequence encoding a polypeptide having an amino acid sequence of SEQ ID NO: 50;
(c) a polynucleotide sequence encoding a polypeptide having an amino acid sequence of SEQ ID NO: 56 and a polynucleotide sequence encoding a polypeptide having an amino acid sequence of SEQ ID NO: 54;
(d) a polynucleotide sequence encoding a polypeptide having an amino acid sequence of SEQ ID NO: 60 and a polynucleotide sequence encoding a polypeptide having an amino acid sequence of SEQ ID NO: 58;
(e) a polynucleotide sequence encoding a polypeptide having an amino acid sequence of SEQ ID NO: 64 and a polynucleotide sequence encoding a polypeptide having an amino acid sequence of SEQ ID NO: 62;
(f) a polynucleotide sequence encoding a polypeptide having an amino acid sequence of SEQ ID NO: 68 and a polynucleotide sequence encoding a polypeptide having an amino acid sequence of SEQ ID NO: 66;
(g) a polynucleotide sequence encoding a polypeptide having an amino acid sequence of SEQ ID NO: 72 and a polynucleotide sequence encoding a polypeptide having an amino acid sequence of SEQ ID NO:70;
(h) a polynucleotide sequence encoding a polypeptide having an amino acid sequence of SEQ ID NO: 76 and a polynucleotide sequence encoding a polypeptide having an amino acid sequence of SEQ ID NO: 74;
(i) a polynucleotide sequence encoding a polypeptide having an amino acid sequence of SEQ ID NO: 80 and a polynucleotide sequence encoding a polypeptide having an amino acid sequence of SEQ ID NO: 78;
(j) a polynucleotide sequence encoding a polypeptide having an amino acid sequence of SEQ ID NO: 84 and a polynucleotide sequence encoding a polypeptide having an amino acid sequence of SEQ ID NO: 82;
wherein the cell is capable of expressing an antibody or antigen-binding fragment thereof that binds human PSMA.

15. A mammalian host cell comprising:
(a) a polynucleotide sequence of SEQ ID NO: 47 and a polynucleotide sequence of SEQ ID NO: 45;
(b) a polynucleotide sequence of SEQ ID NO: 51 and a polynucleotide sequence of SEQ ID NO: 49;
(c) a polynucleotide sequence of SEQ ID NO: 55 and a polynucleotide sequence of SEQ ID NO: 53;
(d) a polynucleotide sequence of SEQ ID NO: 59 and a polynucleotide sequence of SEQ ID NO: 57;
(e) a polynucleotide sequence of SEQ ID NO: 63 and a polynucleotide sequence of SEQ ID NO: 61;
(f) a polynucleotide sequence of SEQ ID NO: 67 and a polynucleotide sequence SEQ ID NO: 65;
(g) a polynucleotide of SEQ ID NO: 71 and a polynucleotide sequence of SEQ ID NO: 69;
(h) a polynucleotide sequence of SEQ ID NO: 75 and a polynucleotide sequence of SEQ ID NO: 73;
(i) a polynucleotide sequence of SEQ ID NO: 79 and a polynucleotide sequence of SEQ ID NO: 77;
(j) a polynucleotide sequence of SEQ ID NO: 83 and a polynucleotide sequence of SEQ ID NO: 81;
wherein the cell is capable of expressing an antibody or antigen-binding fragment thereof that binds human PSMA.

16-17. (canceled)

18. A method of treating cancer comprising administering to a patient in need thereof an effective amount of the antibody or antigen-binding fragment thereof of claim 1.

19. The method of claim 18, wherein the cancer is a solid tumor. (Original) The method of claim 19, wherein the cancer is prostate cancer, lung cancer, sarcoma, breast cancer, kidney cancer, or a digestive tract malignant tumor.

21. The method of claim 18, further comprising administering one or more anti-cancer agents simultaneously, separately, or sequentially.

22. (canceled)

23. A recombinant PSMA polypeptide that does not include residues 418-567 of full-length PSMA as set forth in SEQ ID NO: 85.

24-42. (canceled)
